(19) United States
(12) Patent Application Publication
(10) Pub. No.: US 2026/0176589 A1
(43) Pub. Date: Jun. 25, 2026

(54) CULTURE MEDIA AND CONDITIONS FOR IN VITRO EXPANSION OF HEPATOCYTES

(71) Applicant: Satellite Biosciences, Inc., Watertown, MA (US)

(72) Inventors: Julie MORSE, Acton, MA (US); Joseph E. MARTURANO, Bedford, MA (US); Sunil Kumar MALLANNA, Natick, MA (US); Soujanya Sathyanarayana KARANTH, Natick, MA (US); Arnav CHHABRA, Westborough, MA (US); David C. BRODERICK, Brookline, MA (US); William SCHOLL, Cambridge, MA (US); Aarohi THAKKAR, Everett, MA (US); Thomas Jay LOWERY, JR., Sudbury, MA (US)

(21) Appl. No.: 19/060,439

(22) Filed: Feb. 21, 2025

Related U.S. Application Data
(60) Provisional application No. 63/736,374, filed on Dec. 19, 2024.

(51) Int. Cl. C12N 5/071

(52) U.S. Cl. CPC C12N 5/067; C12N 2501/11; C12N 2501/119; C12N 2501/12; C12N 2501/15; C12N 2506/45; C12N 2533/52

(57) ABSTRACT

Provided herein are completely defined culture conditions, including culture supplements and requirements for an extracellular matrix, for facilitating the expansion of primary human hepatocytes.

### FIELD OF THE INVENTION

The present disclosure relates generally to methods of culturing, expanding, and maturing hepatocytes.

### BACKGROUND OF THE INVENTION

Liver disease (e.g., hepatic disease) is any disease that negatively affects the normal, healthy performance of the liver. The resulting disturbance of liver function causes illness. For example, impaired liver function can result in an accumulation of toxins (e.g., nitrogenous waste compounds) in the blood. These toxins may travel to the brain and affect the nervous system. The Center for Disease Control reports that 4.5 million Americans have been diagnosed with liver disease. While organ replacement therapy can rescue impaired native liver function, the demand far exceeds availability. A feasible alternative is to implant a population of hepatocytes with therapeutic potential into the subject. However, hepatocytes, such as primary human hepatocytes (PHHs), are in short supply. Therefore, improved methods for culturing and expanding hepatocytes, such as PHHs, are needed.

### SUMMARY OF THE INVENTION

The invention provides methods for long-term maintenance, expansion, and maturation of hepatocytes (e.g., primary human hepatocytes (PHHs) or induced pluripotent stem cell (iPSC)-derived hepatocytes), which may be useful for generating a cell-based product for implantation in a subject in need thereof (e.g., a human subject with a liver disorder or a human subject who is at risk of developing a liver disorder) to supplement or rescue native liver function.

In one aspect, the disclosure features a method of culturing hepatocytes, the method including culturing one or more hepatocytes in contact with an extracellular matrix (ECM) in the presence of an expansion medium including a basal cell culture medium for human cells to which is added: a fibroblast growth factor (FGF), an epidermal growth factor (EGF), a hepatocyte growth factor (HGF), an R-spondin, and a transforming growth factor-beta (TGF-beta) inhibitor, wherein one or more of FGF7, Wnt3a, and transforming growth factor-alpha (TGF-alpha) are not added to the culture medium.

In some embodiments, two or more of FGF7, Wnt3a, and TGF-alpha are not added to the basal cell culture medium. In some embodiments, FGF7, Wnt3a, and TGF-alpha are not added to the basal cell culture medium.

In another aspect, the disclosure features a method of culturing one or more hepatocytes on a cell surface in the presence of a cell culture medium including a basal cell culture medium for human cells to which is added a concentration of:
an FGF,
an EGF,
an HGF,
an R-spondin, and
a TGF-beta inhibitor,
   wherein one or more of FGF7, Wnt3a, and TGF-alpha are not added to the cell culture medium, wherein the surface of the cell culture vessel is coated with an ECM, and wherein the hepatocytes are cultured in a two-dimensional culture system.

In some embodiments, one or more of alanine, asparagine, aspartic acid, glutamic acid, glycine, proline, or serine are added to the cell culture medium. In some embodiments, two or more of alanine, asparagine, aspartic acid, glutamic acid, glycine, proline, or serine are added to the cell culture medium. In some embodiments, three or more of alanine, asparagine, aspartic acid, glutamic acid, glycine, proline, or serine are added to the cell culture medium. In some embodiments, four or more of alanine, asparagine, aspartic acid, glutamic acid, glycine, proline, or serine are added to the cell culture medium. In some embodiments, five or more of alanine, asparagine, aspartic acid, glutamic acid, glycine, proline, or serine are added to the cell culture medium. In some embodiments, six or more of alanine, asparagine, aspartic acid, glutamic acid, glycine, proline, or serine are added to the cell culture medium. In some embodiments, alanine, asparagine, aspartic acid, glutamic acid, glycine, proline, and serine are added to the cell culture medium.

In some embodiments,
(a) the concentration of the FGF in the cell culture medium is 5 ng/ml to 100 ng/ml (e.g., 5 to 25 ng/ml, 5 to 50 ng/ml, or 5 to 100 ng/ml; e.g., 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, or 100 ng/ml);
(b) the concentration of the EGF in the cell culture medium is 10 ng/ml to 100 ng/ml (e.g., 10 to 50 ng/ml, 10 to 100 ng/ml, 10 to 100 ng/ml, 25 to 50 ng/ml, or 25 to 100 ng/ml; e.g., 10, 15, 20, 25, 30, 35, 40, 45, 50, 55, 60, 65, 70, 75, 80, 85, 90, 95, or 100 ng/ml);
(c) the concentration of the R-spondin in the cell culture medium is 5 ng/ml to 150 ng/ml (e.g., 5 to 25 ng/ml, 5 to 50 ng/ml, 5 to 100 ng/ml, 5 to 150 ng/ml, 10 to 100 ng/ml, 10 to 150 ng/ml, 50 to 100 ng/ml, or 50 to 150 ng/ml; e.g., 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 130, 140, or 150 ng/ml);
(d) the concentration of the HGF in the cell culture medium is 10 ng/ml to 50 ng/ml (e.g., 10 to 50 ng/mL, 10 to 30 ng/ml, 10 to 50 ng/ml, or 20 to 50 ng/mL; e.g., 10, 11, 12, 13, 14, 15, 16, 17, 18, 19, 20, 21, 22, 23, 24, 25, 26, 27, 28, 29, 30, 31, 32, 33, 34, 35, 40, 45, or 50 ng/ml); or
(e) the TGF-beta inhibitor comprises A83-01, wherein the concentration of A83-01 in the cell culture medium is 1 μM to 5 μM (e.g., 1 to 2 μM, 2 to 3 μM, 3 to 4 μM, or 4 to 5 μM; e.g., 1 μM, 1.1 μM, 1.2μ, 1.3μ, 1.4μ, 1.5μ, 1.6μ, 1.7μ, 1.8μ, 1.9μ, 2μ, 2.1μ, 2.2μ, 2.3μ, 2.4μ, 2.5 μM, 2.6 μM, 2.7 μM, 2.8 μM, 2.9 μM, 3 μM, 3.5 μM, 4 μM, 4.5 μM, or 5 μM).

In some embodiments, a serum or a serum replacement component is added to the cell culture medium at a volumetric concentration of 1% to 6% (e.g., 1%, 2%, 3%, 4%, 5%, or 6%) and is increased to a volumetric concentration of 7% to 15% (e.g., 7%, 8%, 9%, 10%, 11%, 12%, 13%, 14%, or 15%) when the cell density of the hepatocytes reaches 30% to 70% (e.g., 30%, 35%, 40%, 45%, 50%, 55%, 60%, 65%, or 70%) confluency.

In some embodiments, the cell culture vessel is coated with an ECM at a density of 0.1 μg/cm² to 0.7 μg/cm² (e.g., 0.1 to 0.3 μg/cm², 0.2 to 0.5 μg/cm², 0.3 to 0.6 μg/cm², or 0.5 to 0.7 μg/cm²; e.g., 0.1, 0.2, 0.3, 0.4, 0.5, 0.6, or 0.7 μg/cm²). In some embodiments, the ECM comprises a laminin (e.g., laminin-111, laminin-211, laminin-221, laminin-332, laminin-411, laminin-421, laminin-511, laminin-521, or a combination thereof). In some embodiments the ECM does not include a hydrogel or a three-dimensional matrix.

In some embodiments, a B27 supplement, an N2 supplement, N-acetyl cysteine, or a combination thereof is added to the cell culture medium.

In some embodiments, the hepatocytes are primary human hepatocytes (PHHs). In some embodiments, the hepatocytes are derived from reprogrammed cells. In some embodiments, the reprogrammed cells are reprogrammed induced pluripotent stem cells (IPSCs). In some embodiments, the hepatocytes are terminally differentiated hepatocytes.

In another aspect, the disclosure features a method of culturing hepatocytes that includes culturing one or more hepatocytes in contact with an ECM in the presence of a cell culture medium that includes a basal cell culture medium for human cells to which is added a concentration of:
an FGF,
an EGF,
an HGF,
an R-spondin,
and a TGF-beta inhibitor,
wherein one or more of FGF7, Wnt3a, and TGF-alpha and one or more of gastrin, arachidonic acid, and prostaglandin E2 are not added to the cell culture medium.

In some embodiments, two or more of FGF7, Wnt3a, and TGF-alpha are not added to the cell culture medium. In some embodiments, FGF7, Wnt3a, and TGF-alpha are not added to the cell culture medium.

In some embodiments, two or more of gastrin, arachidonic acid, and prostaglandin E2 are not added to the cell culture medium. In some embodiments, gastrin, arachidonic acid, and prostaglandin E2 are not added to the cell culture medium.

In some embodiments, the TGF-beta inhibitor does not comprise Noggin. In some embodiments, the FGF comprises FGF10. In some embodiments, the R-spondin includes R-spondin 1.

In some embodiments,
(a) the concentration of the FGF in the cell culture medium is 5 ng/ml to 100 ng/ml (e.g., 5 to 25 ng/ml, 5 to 50 ng/ml, or 5 to 100 ng/ml; e.g., 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, or 100 ng/ml);
(b) the concentration of the EGF in the cell culture medium is 10 ng/ml to 100 ng/ml (e.g., 10 to 50 ng/ml, 10 to 100 ng/ml, 10 to 100 ng/ml, 25 to 50 ng/ml, or 25 to 100 ng/ml; e.g., 10, 15, 20, 25, 30, 35, 40, 45, 50, 55, 60, 65, 70, 75, 80, 85, 90, 95, or 100 ng/ml);
(c) the concentration of the R-spondin in the cell culture medium is 5 ng/mL to 150 ng/ml (e.g., 5 to 25 ng/ml, 5 to 50 ng/ml, 5 to 100 ng/ml, 5 to 150 ng/ml, 10 to 100 ng/ml, 10 to 150 ng/ml, 50 to 100 ng/ml, or 50 to 150 ng/ml; e.g., 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 130, 140, or 150 ng/ml);
(d) the concentration of the HGF in the cell culture medium is 10 ng/ml to 50 ng/ml (e.g., 10 to 50 ng/ml, 10 to 30 ng/ml, 10 to 50 ng/ml, or 20 to 50 ng/ml; e.g., 10, 11, 12, 13, 14, 15, 16, 17, 18, 19, 20, 21, 22, 23, 24, 25, 26, 27, 28, 29, 30, 31, 32, 33, 34, 35, 40, 45, or 50 ng/ml); or
(e) the TGF-beta inhibitor comprises A83-01, wherein the concentration of A83-01 in the cell culture medium is 1 μM to 5 μM (e.g., 1 to 2 μM, 2 to 3 μM, 3 to 4 μM, or 4 to 5 μM; e.g., 1 μM, 1.1 μM, 1.2 μM, 1.3 μM, 1.4 μM, 1.5 μM, 1.6 μM, 1.7 μM, 1.8 μM, 1.9 μM, 2 μM, 2.1 μM, 2.2 μM, 2.3 μM, 2.4 μM, 2.5 μM, 2.6 μM, 2.7 μM, 2.8 μM, 2.9 μM, 3 μM, 3.5 μM, 4 μM, 4.5 μM, or 5 M).

In some embodiments, one or more of alanine, asparagine, aspartic acid, glutamic acid, glycine, proline, or serine are added to the cell culture medium. In some embodiments, two or more of alanine, asparagine, aspartic acid, glutamic acid, glycine, proline, or serine are added to the cell culture medium. In some embodiments, three or more of alanine, asparagine, aspartic acid, glutamic acid, glycine, proline, or serine are added to the cell culture medium. In some embodiments, four or more of alanine, asparagine, aspartic acid, glutamic acid, glycine, proline, or serine are added to the cell culture medium. In some embodiments, five or more of alanine, asparagine, aspartic acid, glutamic acid, glycine, proline, or serine are added to the cell culture medium. In some embodiments, six or more of alanine, asparagine, aspartic acid, glutamic acid, glycine, proline, or serine are added to the cell culture medium. In some embodiments, alanine, asparagine, aspartic acid, glutamic acid, glycine, proline, and serine are added to the cell culture medium.

In some embodiments, the ECM is coated on a surface, and the hepatocytes are cultured on the ECM-coated surface.

In some embodiments, the hepatocytes are PHHs. In some embodiments, the hepatocytes are derived from reprogrammed cells. In some embodiments, the reprogrammed cells are iPSCs. In some embodiments, the hepatocytes are terminally differentiated hepatocytes.

In another aspect, the disclosure features a method of culturing hepatocytes in contact with an ECM in the presence of a cell culture medium, wherein the hepatocytes are plated and expanded, wherein a serum or serum replacement component is added to the cell culture medium at a volumetric concentration of 1% to 6% (e.g., 1%, 2%, 3%, 4%, 5%, or 6%) upon plating the hepatocytes and is increased in the cell culture medium to a volumetric concentration of 7% to 15% (e.g., 7%, 8%, 9%, 10%, 11%, 12%, 13%, 14%, or 15%) over the course of culturing, and wherein the cell culture medium comprises a basal cell culture medium for human cells to which is added a concentration of:
an FGF,
an EGF,
an HGF,
an R-spondin, and
a TGF-beta inhibitor,
   wherein one or more of FGF7, Wnt3a, and TGF-alpha are not added to the cell culture medium.

In some embodiments, two or more of FGF7, Wnt3a, and TGF-alpha are not added to the cell culture medium. In some embodiments, FGF7, Wnt3a, and TGF-alpha are not added to the cell culture medium.

In some embodiments, the method comprises passaging the hepatocytes, wherein each passage of 5 to 25 days (e.g., 5 to 7 days, 5 to 10 days, 5 to 14 days, 5 to 20 days, 5 to 25 days, 7 to 10 days, 7 to 14 days, 10 to 15 days, 10 to 20 days, 10 to 25 days, 14 to 20 days, 14 to 25 days, or 20 to 25 days; e.g., 5, 6, 7, 8, 9, 10, 11, 12, 13, 14, 15, 16, 17, 18, 19, 20, 21, 22, 23, 24, or 25 days).

In some embodiments, the serum or serum replacement component is increased to a volumetric concentration of 7% to 15% (e.g., 7%, 8%, 9%, 10%, 11%, 12%, 13%, 14%, or 15%) when the cell density of the hepatocytes reaches 30 to 70% (e.g., 30%, 35%, 40%, 45%, 50%, 55%, 60%, 65%, or 70%) confluency.

In some embodiments, one or more of alanine, asparagine, aspartic acid, glutamic acid, glycine, proline, or serine are added to the cell culture medium. In some embodiments, two or more of alanine, asparagine, aspartic acid, glutamic acid, glycine, proline, or serine are added to the cell culture medium. In some embodiments, three or more of alanine, asparagine, aspartic acid, glutamic acid, glycine, proline, or serine are added to the cell culture medium. In some embodiments, four or more of alanine, asparagine, aspartic acid, glutamic acid, glycine, proline, or serine are added to the cell culture medium. In some embodiments, five or more of alanine, asparagine, aspartic acid, glutamic acid, glycine, proline, or serine are added to the cell culture medium. In some embodiments, six or more of alanine, asparagine, aspartic acid, glutamic acid, glycine, proline, or serine are added to the cell culture medium. In some embodiments, alanine, asparagine, aspartic acid, glutamic acid, glycine, proline, and serine are added to the cell culture medium.

In some embodiments, the ECM comprises a laminin (e.g., laminin-111, laminin-211, laminin-221, laminin-332, laminin-411, laminin-421, laminin-511, laminin-521, or a combination thereof).

In some embodiments, the ECM is coated on a culture surface, and the hepatocytes are cultured on the ECM-coated surface.

In some embodiments, the hepatocytes are PHHs. In some embodiments, the hepatocytes are derived from reprogrammed cells. In some embodiments, the reprogrammed cells are iPSCs. In some embodiments, the hepatocytes are terminally differentiated hepatocytes.

In some embodiments,
(a) the concentration of the FGF in the cell culture medium is 5 ng/ml to 100 ng/ml (e.g., 5 to 25 ng/ml, 5 to 50 ng/ml, or 5 to 100 ng/ml; e.g., 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, or 100 ng/ml);
(b) the concentration of the EGF in the cell culture medium is 10 ng/ml to 100 ng/ml (e.g., 10 to 50 ng/ml, 10 to 100 ng/ml, 10 to 100 ng/ml, 25 to 50 ng/ml, or 25 to 100 ng/ml; e.g., 10, 15, 20, 25, 30, 35, 40, 45, 50, 55, 60, 65, 70, 75, 80, 85, 90, 95, or 100 ng/ml);
(c) the concentration of the R-spondin in the cell culture medium is 5 ng/ml to 150 ng/ml (e.g., 5 to 25 ng/ml, 5 to 50 ng/mL, 5 to 100 ng/ml, 5 to 150 ng/ml, 10 to 100 ng/ml, 10 to 150 ng/ml, 50 to 100 ng/mL, or 50 to 150 ng/ml; e.g., 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 130, 140, or 150 ng/ml);
(d) the concentration of the HGF in the cell culture medium is 10 ng/ml to 50 ng/mL (e.g., 10 to 50 ng/ml, 10 to 30 ng/ml, 10 to 50 ng/ml, or 20 to 50 ng/ml; e.g., 10, 11, 12, 13, 14, 15, 16, 17, 18, 19, 20, 21, 22, 23, 24, 25, 26, 27, 28, 29, 30, 31, 32, 33, 34, 35, 40, 45, or 50 ng/ml); or
(e) the TGF-beta inhibitor comprises A83-01, wherein the concentration of A83-01 in the cell culture medium is 1 μM to 5 μM (e.g., 1 to 2 μM, 2 to 3 μM, 3 to 4 μM, or 4 to 5 μM; e.g., 1 μM, 1.1 μM, 1.2 μM, 1.3 μM, 1.4 μM, 1.5 μM, 1.6 μM, 1.7 μM, 1.8 μM, 1.9 μM, 2 μM, 2.1 μM, 2.2 μM, 2.3 μM, 2.4 μM, 2.5 μM, 2.6 μM, 2.7 μM, 2.8 μM, 2.9 μM, 3 μM, 3.5 μM, 4 μM, 4.5 μM, or 5 μM).

In some embodiments, one or more of gastrin, arachidonic acid, and prostaglandin E2 are not added to the cell culture medium. In some embodiments, two or more of gastrin, arachidonic acid, and prostaglandin E2 are not added to the cell culture medium. In some embodiments, gastrin, arachidonic acid, and prostaglandin E2 are not added to the cell culture medium.

In some embodiments, the TGF-beta inhibitor does not include Noggin. In some embodiments, the FGF comprises FGF10. In some embodiments, the R-spondin includes R-spondin 1.

In another aspect, the disclosure features a method of culturing hepatocytes, the method including culturing one or more hepatocytes in contact with an ECM in the presence of an expansion medium including a basal cell culture medium for human cells to which is added: an FGF, an HGF, TGF-alpha, and a TGF-beta inhibitor, wherein one or more of FGF7, an EGF, Wnt3a, and an R-spondin are not added to the culture medium.

In some embodiments, two or more of FGF7, an EGF, Wnt3a, and an R-spondin are not added to the culture medium. In some embodiments, three or more of FGF7, an EGF, Wnt3a, and an R-spondin are not added to the culture medium. In some embodiments, FGF, an EGF, Wnt3a, and an R-spondin are not added to the culture medium.

In some embodiments of any of the foregoing aspects, the FGF added to the basal cell culture medium is not FGF7.

In some embodiments, the R-spondin includes R-spondin 1, R-spondin 2, R-spondin 3, R-spondin 4, or a combination thereof. In some embodiments, the R-spondin includes R-spondin 1.

In some embodiments, the method further includes adding an antioxidant to the basal cell culture medium. In some embodiments, the antioxidant includes N-acetyl cysteine, nicotinamide, or a combination thereof.

In some embodiments, the method further includes adding a cell survival agent to the basal cell culture medium. In some embodiments, the cell survival agent is a supplement for a serum-free cell medium. In some embodiments, the supplement includes a B27 supplement, an N2 supplement, or a combination thereof. In some embodiments, the supplement does not include vitamin A.

In some embodiments, the FGF includes FGF2, FGF3, FGF10, FGF22, or a combination thereof. In some embodiments, the FGF includes FGF10.

In some embodiments, the TGF-beta inhibitor includes an inhibitor of activin receptor-like kinase 5 (ALK5). In some embodiments, the TGF-beta inhibitor includes A83-01.

In some embodiments, the method further includes adding an amino acid supplement to the basal cell culture medium. In some embodiments, the amino acid supplement includes a non-essential amino acid (NEAA) supplement, an L-glutamine substitute, or a combination thereof.

In some embodiments, the method further includes adding a serum replacement component to the basal cell culture medium. In some embodiments, the serum replacement component includes KNOCKOUT™ Serum Replacement (KOSR).

In some embodiments, the method further includes adding a buffering agent to the basal cell culture medium. In some embodiments, the buffering agent includes N-2-hydroxyethylpiperazine-N′-2′-ethanesulfonic acid (HEPES).

In some embodiments, the ECM includes a laminin, a collagen, a vitronectin, a fibronectin, or a combination thereof. In some embodiments, the ECM includes a xeno-free substrate. In some embodiments, the laminin is laminin-111, laminin-211, laminin-221, laminin-332, laminin-411, laminin-421, laminin-511, or laminin-521. In some embodiments, the laminin is laminin-521. In some embodiments, the ECM is applied to a surface. In some embodiments, the ECM is applied to the surface at a density of 0.1 μg/cm² to 0.7 μg/cm² (e.g., 0.1, 0.105, 0.11, 0.115, 0.12, 0.125, 0.13, 0.135, 0.14, 0.145, 0.15, 0.16, 0.17, 0.18, 0.19, 0.2, 0.21, 0.22, 0.23, 0.24, 0.25, 0.3, 0.35, 0.4, 0.45, 0.5, 0.55, 0.6, 0.65, or 0.7 g/cm²). In some embodiments, the ECM is applied to the surface at a density of 0.125 g/cm².

In some embodiments, the hepatocytes are PHHs. In some embodiments, the hepatocytes are derived from reprogrammed cells. In some embodiments, the hepatocytes are iPSC-derived hepatocytes. In some embodiments, the PHHs were previously cryopreserved. In some embodiments, the PHHs have not been previously cryopreserved.

In some embodiments, the method includes expanding plated hepatocytes (step P0) and a first passage of expanded hepatocytes (step P1).

In some embodiments, step P0 has a duration of 5 to 25 days, e.g., 10 to 16 days (e.g., 5, 6, 7, 8, 9, 10, 11, 12, 13, 14, 15, 16, 17, 18, 19, 20, 21, 22, 23, 24, or 25 days). In some embodiments, step P0 has a duration of 10 to 14 days (e.g., 10, 11, 12, 13, or 14 days). In some embodiments, the expansion medium is replaced every 24 to 48 hours or 24 to 72 hours during step P0. In some embodiments, at step P0, the hepatocytes are seeded at a cell density of 300 viable cells/cm² to 13,000 viable cells/cm². In some embodiments, step P1 has a duration of 5 to 25 days, e.g., 7 to 16 days (e.g., 5, 6, 7, 8, 9, 10, 11, 12, 13, 14, 15, 16, 17, 18, 19, 20, 21, 22, 23, 24, or 25 days). In some embodiments, step P1 has a duration of 10 to 14 days (e.g., 10, 11, 12, 13, or 14 days). In some embodiments, the expansion medium is replaced every 24 to 48 hours or 24 to 72 hours during step P1. In some embodiments, at step P1, the hepatocytes are seeded at a cell density of 300 viable cells/cm² to 13,000 viable cells/cm² (e.g., 300, 400, 500, 600, 700, 800, 900, 1,000, 2,000, 3,000, 4,000, 5,000, 6,000, 7,000, 8,000, 9,000, 10,000, 11,000, 12,000, or 13,000 viable cells/cm²)

In some embodiments, step P0 and step P1 have a total duration of 10 to 50 days, e.g., 20 days to 30 days (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days).

In some embodiments, the population of hepatocytes expand by at least 10-fold or at least 100-fold, e.g., 10-fold to 200-fold during step P0 (e.g., about 10-fold, about 15-fold, about 20-fold, about 25-fold, about 30-fold, about 35-fold, about 40-fold, about 45-fold, about 50-fold, about 60-fold, about 70-fold, about 80-fold, about 90-fold, about 100-fold, about 110-fold, about 120-fold, about 130-fold, about 140-fold, about 150-fold, about 160-fold, about 170-fold, about 180-fold, about 190-fold, or about 200-fold) and/or by at least 500-fold or at least 1,000-fold, e.g., 500-fold to 10,000-fold cumulatively in step P0 and step P1 (e.g., 500-fold to 1,000-fold, 1,000-fold to 2,000-fold, 1,000-fold to 5,000-fold, 2,000-fold to 3,000-fold, 3,000-fold to 4,000-fold, 4,000-fold to 5,000-fold, 5,000-fold to 6,000-fold, 5,000-fold to 10,000-fold, 6,000-fold to 7,000-fold, 7,000-fold to 8,000-fold, 8,000-fold to 9,000-fold, 8,000-fold to 10,000-fold, or 9,000-fold to 10,000-fold).

In some embodiments, at least 70% of the hepatocytes (e.g., at least 70%, at least 75%, at least 80%, at least 85%, at least 90%, or at least 95%) are viable following the culturing.

In some embodiments, the method includes culturing the hepatocytes in a maturation medium including a basal cell culture medium for human cells to which one or more maturation supplements are added. In some embodiments, the hepatocytes are cultured in the maturation medium for a duration of 3 days to 7 days (e.g., 3, 4, 5, 6, or 7 days). In some embodiments, the hepatocytes are cultured in the maturation medium for a duration of 4 days. In some embodiments, culturing in the maturation medium begins immediately after hepatocyte expansion. In some embodiments, the expanded hepatocytes have been cryopreserved prior to culturing in the maturation medium.

In some embodiments, the basal cell medium is selected from the group consisting of Takara Cellartis POWER™ Primary HEP medium, Lonza HCM™ cell culture medium, William's E medium, and LIFENET HEALTH® Human Hepatocyte Media.

In some embodiments, the one or more maturation supplements include HEPES, an L-glutamine substitute, insulin transferrin selenium, a Notch inhibitor, an EGFR inhibitor, Oncostatin M, an antioxidant, a glucocorticoid, a pregnane X receptor (PXR) activator, a bile acid, a cAMP or cAMP analog, cholesterol, a thyroid hormone, a serum replacement component, or any combination thereof.

In some embodiments, the Notch inhibitor includes Compound E, Gamma Secretase Inhibitor XX, or a combination thereof. In some embodiments, the EGFR inhibitor includes erlotinib HCl. In some embodiments, the antioxidant includes vitamin C. In some embodiments, the glucocorticoid includes dexamethasone, hydrocortisone, or a combination thereof. In some embodiments, the PXR activator is vitamin K2, lithocholic acid, or a combination thereof. In some embodiments, the bile acid is lithocholic acid, urso deoxycholic acid, or a combination thereof. In some embodiments, the CAMP analog is 8-bromo CAMP, forskolin, or a combination thereof. In some embodiments, the thyroid hormone is T3.

In some embodiments, the method includes adding an amino acid supplement to the basal cell culture medium. In some embodiments, the amino acid supplement includes an NEAA supplement, an essential amino acid (EAA) supplement, or a combination thereof.

In some embodiments, the method includes adding a peptide hormone to the basal cell culture medium. In some embodiments, the peptide hormone includes glucagon.

In some embodiments, the method includes adding a DNase to the basal cell culture medium.

In some embodiments, the method includes adding a rho kinase (ROCK) inhibitor.

In some embodiments, the method includes adding a non-ionic detergent. In some embodiments, the non-ionic detergent includes Pluronic F68.

In some embodiments, the method includes determining the expression profile of the hepatocytes following culturing.

In some embodiments, the expression profile of the hepatocytes includes expression of one or more biomarkers selected from hepatocyte nuclear factor 4 alpha (HNF4-alpha), cluster of differentiation 81 (CD81), asialoglycoprotein receptor 1 (ASGR1), nuclear receptor subfamily 1 group I member 2 (NR112), alpha-1 antitrypsin (A1AT), cytochrome P450 family 3 subfamily A member 4 (CYP3A4), cytochrome P450 family 1 subfamily A member 2 (CYP1A2), factor IX, and/or albumin by at least 70% of the hepatocytes (e.g., at least 70%, at least 75%, at least 80%, at least 85%, at least 90%, or at least 95%). In some embodiments, the expression profile of the hepatocytes includes expression of one or more biomarkers selected from HNF4-alpha, CD81, ASGPR1, NR112, A1AT, CYP3A4, CYP1A2, factor IX, and/or albumin by at least 80% of the hepatocytes (e.g., at least 80%, at least 85%, at least 90%, or at least 95%).

In some embodiments, the expression profile of the hepatocytes includes expression of one or more urea cycle enzymes by at least 70% of the hepatocytes (e.g., at least 70%, at least 75%, at least 80%, at least 85%, at least 90%, or at least 95%). In some embodiments, the expression profile of the hepatocytes includes expression of one or more urea cycle enzymes by at least 80% of the hepatocytes (e.g., at least 80%, at least 85%, at least 90%, or at least 95%). In some embodiments, the one or more urea cycle enzymes include arginase 1 (ARG1), argininosuccinate lyase (ASL), argininosuccinate synthase 1 (ASS1), carbamoyl-phosphate synthase 1 (CPS1), N-acetylglutamate synthase (NAGS), ornithine transcarbamylase (OTC), or a combination thereof.

In some embodiments, the expression profile of the hepatocytes includes expression of one or more progenitor markers and/or cholangiocyte markers by 10% or fewer of the hepatocytes (e.g., 10% or fewer, 9% or fewer, 8% or fewer, 7% or fewer, 6% or fewer, or 5% or fewer). In some embodiments, the one or more progenitor markers include alpha-fetoprotein (AFP), leucine rich repeat containing G protein-coupled receptor 5 (LGR5), cytochrome P450 family 3 subfamily A member 7 (CYP3A7), epithelial cell adhesion molecule (EpCAM), T-box transcription factor (TBX3), or a combination thereof. In some embodiments, the one or more cholangiocyte markers include aquaporin 1 (AQP1), keratin 19 (KRT19), keratin 7 (KRT7), trefoil factor 1 (TFF1), trefoil factor 2 (TFF2), or a combination thereof.

In another aspect, the disclosure features a method of maturing hepatocytes (e.g., PHHs or iPSC-derived hepatocytes), the method including culturing hepatocytes in a maturation medium including a basal cell culture medium for human cells to which one or more maturation supplements are added. In some embodiments, the hepatocytes are cultured in the maturation medium for a duration of 3 days to 7 days (e.g., 3, 4, 5, 6, or 7 days). In some embodiments, the hepatocytes are cultured in the maturation medium for a duration of 4 days.

In some embodiments, the basal cell medium is selected from the group consisting of Takara Cellartis POWER™ Primary HEP medium, Lonza HCM™ cell culture medium, William's E medium, and LIFENET HEALTH® Human Hepatocyte Media.

In some embodiments, the one or more maturation supplements include HEPES, an L-glutamine substitute, insulin transferrin selenium, a Notch inhibitor, an EGFR inhibitor, Oncostatin M, an antioxidant, a glucocorticoid, a PXR activator, a bile acid, a cAMP or cAMP analog, cholesterol, a thyroid hormone, a serum replacement component, an amino acid supplement, a peptide hormone, or any combination thereof.

In some embodiments, the Notch inhibitor includes Compound E, Gamma Secretase Inhibitor XX, or a combination thereof. In some embodiments, the EGFR inhibitor includes erlotinib HCl. In some embodiments, the antioxidant includes vitamin C. In some embodiments, the glucocorticoid includes dexamethasone, hydrocortisone, or a combination thereof. In some embodiments, the PXR activator is vitamin K2, lithocholic acid, or a combination thereof. In some embodiments, the bile acid is lithocholic acid, urso deoxycholic acid, or a combination thereof. In some embodiments, the CAMP analog is 8-bromo CAMP, forskolin, or a combination thereof. In some embodiments, the thyroid hormone is T3. In some embodiments, the amino acid supplement includes an NEAA supplement, an EAA supplement, or a combination thereof. In some embodiments, the peptide hormone includes glucagon.

In some embodiments, the method includes determining the expression profile of the hepatocytes following culturing.

In some embodiments, the expression profile of the hepatocytes includes expression of one or more biomarkers selected from HNF4-alpha, CD81, ASGR1, NR112, A1AT, CYP3A4, CYP1A2, factor IX, prothrombin, and albumin by at least 70% of the hepatocytes (e.g., at least 70%, at least 75%, at least 80%, at least 85%, at least 90%, or at least 95%). In some embodiments, the expression profile of the hepatocytes includes expression of one or more biomarkers selected from HNF4-alpha, CD81, ASGR1, NR112, A1AT, CYP3A4, CYP1A2, factor IX, prothrombin, and albumin by at least 80% of the hepatocytes (e.g., at least 80%, at least 85%, at least 90%, or at least 95%).

In some embodiments, the expression profile of the hepatocytes includes expression of one or more urea cycle enzymes by at least 70% of the hepatocytes (e.g., at least 70%, at least 75%, at least 80%, at least 85%, at least 90%, or at least 95%). In some embodiments, the expression profile of the hepatocytes further includes expression of one or more urea cycle enzymes by at least 80% of the hepatocytes (e.g., at least 80%, at least 85%, at least 90%, or at least 95%). In some embodiments, the one or more urea cycle enzymes include ARG1, ASL, ASS1, CPS1, NAGS, OTC, or a combination thereof.

In some embodiments, expression profile of the hepatocytes includes expression of one or more progenitor markers and/or cholangiocyte markers by 10% or fewer of the hepatocytes (e.g., 10% or fewer, 9% or fewer, 8% or fewer, 7% or fewer, 6% or fewer, or 5% or fewer). In some embodiments, the one or more progenitor markers include AFP, LGR5, CYP3A7, EpCAM, TBX3, or a combination thereof. In some embodiments, the one or more cholangiocyte markers include AQP1, KRT19, KRT7, TFF1, TFF2, or a combination thereof.

In some embodiments, prior to maturing the hepatocytes, the hepatocytes are cultured according to any one of the embodiments of the first or second aspect.

In some embodiments, prior to maturing the hepatocytes, the hepatocytes are cultured in contact with an ECM in the presence of an expansion medium including a basal cell culture medium for human cells to which is added: an FGF, an HGF, and a TGF-beta inhibitor.

In some embodiments, the method includes adding an R-spondin to the basal cell culture medium. In some embodiments, the R-spondin includes R-spondin 1, R-spondin 2, R-spondin 3, R-spondin 4, or a combination thereof. In some embodiments, the R-spondin includes R-spondin 1.

In some embodiments, the FGF includes FGF2, FGF3, FGF7, FGF10, FGF22, or a combination thereof.

In some embodiments, the method includes adding an EGF and/or Wnt3a to the basal cell culture medium. In some embodiments, the method includes adding TGF-alpha to the basal cell culture medium.

In some embodiments, the method includes adding an antioxidant to the basal cell culture medium. In some embodiments, the antioxidant includes N-acetyl cysteine, nicotinamide, or a combination thereof.

In some embodiments, the method includes adding a cell survival agent to the basal cell culture medium. In some embodiments, the cell survival agent is a supplement for a serum-free cell medium. In some embodiments, the supplement includes a B27 supplement, an N2 supplement, or a combination thereof. In some embodiments, the supplement does not include vitamin A.

In some embodiments, the TGF-beta inhibitor is an ALK5 inhibitor. In some embodiments, the ALK5 inhibitor is A83-01.

In some embodiments, the method includes adding an amino acid supplement to the basal cell culture medium. In some embodiments, the amino acid supplement includes an NEAA supplement, an L-glutamine substitute, or a combination thereof.

In some embodiments, the method includes adding a serum replacement component to the basal cell culture medium. In some embodiments, the serum replacement component includes KOSR.

In some embodiments, the method includes adding a buffering agent to the basal cell culture medium. In some embodiments, the buffering agent includes HEPES.

In some embodiments, the ECM includes a laminin, a collagen, a vitronectin, a fibronectin, or a combination thereof. In some embodiments, the ECM includes a xeno-free substrate. In some embodiments, the laminin is laminin-111, laminin-211, laminin-221, laminin-332, laminin-411, laminin-421, laminin-511, or laminin-521. In some embodiments, the laminin is laminin-521.

In some embodiments, the ECM is applied to a surface. In some embodiments, the ECM is applied to the surface at a density of 0.1 μg/cm² to 0.7 g/cm².

In some embodiments, the hepatocytes following culturing are administered to a liver of a subject.

In another aspect, the disclosure features a cell culture medium including a basal cell culture medium for human cells and an exogenous source of an FGF, an EGF, an HGF, an R-spondin, and a TGF-beta inhibitor, wherein the cell culture medium does not include an exogenous source of one or more of FGF7, Wnt3a, and TGF-alpha.

In some embodiments of the fourth aspect, the cell culture medium does not include an exogenous source of two or more of FGF7, Wnt3a, and TGF-alpha. In some embodiments, the cell culture medium does not include an exogenous source of FGF7, Wnt3a, and TGF-alpha.

In another aspect, the disclosure features a cell culture medium including a basal cell culture medium for human cells and an exogenous source of an FGF, an HGF, a TGF-alpha, and a TGF-beta inhibitor, wherein the cell culture medium does not include an exogenous source of one or more of FGF7, an EGF, Wnt3a, and an R-spondin.

In some embodiments of the fifth aspect, the cell culture medium does not include an exogenous source of two or more of FGF7, an EGF, Wnt3a, and an R-spondin. In some embodiments, the cell culture medium does not include an exogenous source of three or more of FGF7, an EGF, Wnt3a, and an R-spondin. In some embodiments, the cell culture medium does not include an exogenous source of FGF7, an EGF, Wnt3a, and an R-spondin.

In some embodiments of the fourth or fifth aspect, the FGF is not FGF7.

In some embodiments, the R-spondin includes R-spondin 1, R-spondin 2, R-spondin 3, R-spondin 4, or a combination thereof. In some embodiments, the R-spondin includes R-spondin 1.

In some embodiments, the cell culture medium includes an antioxidant. In some embodiments, the antioxidant includes N-acetyl cysteine, nicotinamide, or a combination thereof.

In some embodiments, the cell culture medium includes a cell survival agent. In some embodiments, the cell survival agent is a supplement for a serum-free cell medium. In some embodiments, the supplement includes a B27 supplement, an N2 supplement, or a combination thereof. In some embodiments, the supplement does not include vitamin A.

In some embodiments, the FGF includes FGF2, FGF3, FGF10, FGF22, or a combination thereof. In some embodiments, the FGF includes FGF10.

In some embodiments, the TGF-beta inhibitor includes an inhibitor of ALK5. In some embodiments, the TGF-beta inhibitor includes A83-01.

In some embodiments, the cell culture medium includes an amino acid supplement. In some embodiments, the amino acid supplement includes an NEAA supplement, an L-glutamine substitute, or a combination thereof.

In some embodiments, the cell culture medium includes a serum replacement component. In some embodiments, the serum replacement component includes KOSR.

In some embodiments, the cell culture medium includes a buffering agent. In some embodiments, the buffering agent includes HEPES.

In another aspect, the disclosure features using the cell culture medium of any one of the foregoing embodiments for expanding a population of hepatocytes (e.g., PHHs or iPSC-derived hepatocytes).

### BRIEF DESCRIPTION OF THE DRAWINGS

FIG. 1 is a series of micrographs showing images of three lots of primary human hepatocytes (PHHs) on day 9 of culturing using Expand 3.0, Expand 4.0, or Expand 5.0 expansion medium formulations.

FIGS. 2A-2C are bar graphs showing the fold expansion of PHHs following thirteen days of expansion in Expand 3.0, Expand 4.0, or Expand 5.0 expansion medium formulations in PHH lot 1 (FIG. 2A), PHH lot 2 (FIG. 2B), and PHH lot 3 (FIG. 2C).

FIGS. 3A-3C are bar graphs depicting the percentage of viable hepatocytes following thirteen days of expansion in Expand 3.0, Expand 4.0, or Expand 5.0 expansion medium formulations in PHH lot 1 (FIG. 3A), PHH lot 2 (FIG. 3B), and PHH lot 3 (FIG. 3C).

FIG. 4 is a series of violin plots depicting mRNA expression levels of hepatocyte lineage biomarkers in PHHs cultured in Expand 3.0, Expand 4.0, or Expand 5.0 expansion medium as determined by RNA-seq. Shown are transcript levels normalized to PHHs for hepatocyte lineage biomarkers albumin (ALB), cytochrome P450 1A2 (CYP1A2), cytochrome P450 3A4 (CYP3A4), hepatocyte nuclear factor 4 alpha (HNF4A), nuclear receptor subfamily 1 group I member 2 (NR112), and serpin family A member 1 (SERPINA1).

FIG. 5 is a series of violin plots depicting mRNA expression levels of urea cycle enzymes in PHHs cultured in Expand 3.0, Expand 4.0, or Expand 5.0 expansion medium as determined by RNA-seq. Shown are transcript levels normalized to PHHs for urea cycle enzymes arginase 1 (ARG1), argininosuccinate lyase (ASL), argininosuccinate synthase 1 (ASS1), carbamoyl phosphate synthetase 1 (CPS1), N-acetylglutamate synthase (NAGS), and ornithine transcarbamylase (OTC).

FIG. 6 is a series of violin plots depicting mRNA expression levels of cell polarity biomarkers in PHHs cultured in Expand 3.0, Expand 4.0, or Expand 5.0 expansion medium as determined by RNA-seq. Shown are transcript levels normalized to PHHs for cell polarity biomarkers ATP binding cassette subfamily B member 11 (ABCB11), ATP binding cassette subfamily C member 2 (ABCC2), ATP binding cassette subfamily C member 3 (ABCC3), ATP binding cassette subfamily G member 2 (ABCG2), scavenger receptor class B member 1 (SCARB1), and solute carrier family 10 member 1 (SLC10A1).

FIG. 7 is a series of violin plots depicting mRNA expression levels of hepatocyte progenitor biomarkers in PHHs cultured in Expand 3.0, Expand 4.0, or Expand 5.0 expansion medium as determined by RNA-seq. Shown are transcript levels normalized to PHHs for hepatocyte progenitor biomarkers alpha fetoprotein (AFP), cytochrome P450 family 3 subfamily A member 7 (CYP3A7), epithelial cellular adhesion molecule (EPCAM), leucine rich repeat containing G protein-coupled receptor 5 (LGR5), and T-box transcription factor 3 (TBX3).

FIG. 8 is a series of violin plots depicting mRNA expression levels of cholangiocyte biomarkers in PHHs cultured in Expand 3.0, Expand 4.0, or Expand 5.0 expansion medium as determined by RNA-seq. Shown are transcript levels normalized to PHHs for cholangiocyte biomarkers aquaporin 1 (AQP1), keratin 19 (KRT19), keratin 7 (KRT7), trefoil factor 1 (TFF1), and trefoil factor 2 (TFF2).

FIG. 9 is a series of violin plots depicting mRNA expression levels of epithelial cell biomarkers in PHHs cultured in Expand 3.0, Expand 4.0, or Expand 5.0 expansion medium as determined by RNA-seq. Shown are transcript levels normalized to PHHs for epithelial cell biomarkers chromodomain helicase DNA binding protein 1 (CHD1) and tight junction protein 1 (TJP1).

FIG. 10 is a series of violin plots depicting mRNA expression levels of mesenchymal cell biomarkers in PHHs cultured in Expand 3.0, Expand 4.0, or Expand 5.0 expansion medium as determined by RNA-seq. Shown are transcript levels normalized to PHHs for mesenchymal cell biomarkers smooth muscle α2 actin (ACTA2) and vimentin (VIM).

FIG. 11 is a graph depicting the expansion medium cost per liter in United States dollar (USD) and the mean fold expansion of three different lots of hepatocytes for sixteen different expansion medium formulations including the Expand 3.0, Expand 4.0, and Expand 5.0 expansion media.

FIG. 12 is series of graphs depicting fold expansion of hepatocytes as modeled by an ordinary least squares (OLS) model when the indicated growth factors or supplements are added or omitted from the cell culture medium. 0: omission of the indicated factor; 1: addition of the indicated factor.

FIG. 13 is a series of micrographs showing two representative images of populations of PHHs that were cultured for 13 days in the indicated cell culture medium formulations.

FIGS. 14A and 14B are representative micrographs of PHHs cultured in the presence of Expand 1.0 expansion medium (FIG. 14A) or Condition 5 expansion medium (FIG. 14B) for two passages.

FIG. 15 is a series of micrographs showing the initial seeding of the first passage (top row) and day 14 of the first passage (bottom row) of PHHs cultured in Expand 1.0 expansion medium (left); Condition 5 expansion medium that included a static volumetric concentration of 1% KOSR (middle); or Condition 5 expansion medium in which the added volumetric concentration of KOSR was 1% on days 1-6, 5% on days 7-8, and 10% on days 9-14 of the culture (right).

FIG. 16 is a series of micrographs showing two representative images of 3D cell aggregates of PHHs and CULTREX® cultured in Expand 1.0 expansion medium at Day 1, Day 13, and Day 18 of culturing. Micrographs are shown at 10× zoom.

FIG. 17 is a series of micrographs showing the four representative images of 3D cell aggregates of FIG. 16 at Day 18 at 40× zoom.

FIG. 18 is a series of micrographs showing three different lots of PHHs that were expanded on a substrate of 0.5 μg/cm² laminin-521 in Expand 4.0 expansion medium for 13 days. The cells were seeded at a density of 325 cells/cm², 650 cells/cm², or 1,300 cells/cm².

FIG. 19 is a series of micrographs showing three different lots of PHHs that were expanded on a substrate of 0.125 μg/cm² laminin-521 in Expand 4.0 expansion medium for 13 days. The cells were seeded at a density of 325 cells/cm², 650 cells/cm², or 1,300 cells/cm².

FIG. 20 is a series of micrographs showing three different lots of PHHs that were expanded on a substrate of 0.5 μg/cm² vitronectin in Expand 4.0 expansion medium for 13 days. The cells were seeded at a density of 325 cells/cm², 650 cells/cm², or 1,300 cells/cm².

FIG. 21 is a series of micrographs showing three different lots of PHHs that were expanded on a substrate of 0.125 μg/cm² vitronectin in Expand 4.0 expansion medium for 13 days. The cells were seeded at a density of 325 cells/cm², 650 cells/cm², or 1,300 cells/cm².

FIG. 22 is a series of micrographs showing three different lots of PHHs that were expanded on a substrate of 1:50 CELLstart™ in Expand 4.0 expansion medium for 13 days. The cells were seeded at a density of 325 cells/cm², 650 cells/cm², or 1,300 cells/cm².

FIG. 23 is a series of micrographs showing three different lots of PHHs that were expanded on a substrate of 1:200 CELLstart™ in Expand 4.0 expansion medium for 13 days. The cells were seeded at a density of 325 cells/cm², 650 cells/cm², or 1,300 cells/cm².

FIGS. 24A and 24B show immunofluorescent data in which FIG. 24A is a series of representative immunofluorescent micrographs depicting staining of HNF4a, albumin, both HNF4a and albumin, or a non-targeting isotype antibody control in a population of PHHs that were expanded for 24 days in Expand 4.0 expansion medium (scale bar: 400 μm), and FIG. 24B is a bar graph depicting the quantification of expanded hepatocytes that expressed albumin, HNF4a, or both.

FIG. 25 is a pair of histograms showing hepatocyte fold expansion at Day 0 (top) or Day 1 (bottom) of culturing in expansion medium formulations in which the concentrations of added human FGF-10, Rspo-1, N2 supplement, and B27 supplement are varied. The mean fold expansion of the Expand 4.0 expansion medium is designated by the dotted line.

FIG. 26 is a pair of histograms showing hepatocyte viability at Day 0 (top) or Day 1 (bottom) of culturing in expansion medium formulations in which the concentrations of added human FGF-10, Rspo-1, N2 supplement, and B27 supplement are varied. The mean viability of the Expand 4.0 expansion medium is designated by the dotted line.

FIG. 27 is a series of graphs depicting fold expansion of hepatocytes as shown by an ordinary least squares (OLS) model when the indicated growth factors or supplements are added at variable concentrations at Day 0 (top) or Day 1 (bottom). Each graph shows the calculated Pearson coefficient (r) and p value. B27 concentrations: −1.94=2 mL/L, −1=4 mL/L, 0=11 mL/L, 1=16 mL/L, 1.94=20 mL/L; FGF-10 concentrations: −1.94=5 ng/ml, −1=15 ng/ml, 0=28 ng/ml, 1=39 ng/ml, 1.94=50 ng/ml; N2 concentrations: −1.94=1 mL/L, −1=2 mL/L, 0=6 mL/L, 1=8 mL/L, 1.94=10 mL/L; Rspondin-1 concentrations: −1.94=10 ng/ml, −1=20 ng/ml, 0=55 ng/mL, 1=79 ng/ml, 1.94=100 ng/ml.

FIG. 28 is a schematic diagram providing an experimental overview of hepatocyte culturing prior to CYP3A4 induction. PHHs were seeded at a density of 6.4×10³ cells/well and were cultured in Expand 3.0 expansion medium for thirteen days, thereby expanding into a population of expanded human hepatocytes (eHHs). After expansion, the cell populations were divided into three experimental groups for three-day CYP3A4 induction in Takara Cellartis POWER™ Primary HEP medium. In Group 1, the eHHs underwent CYP3A4 induction immediately after expansion. In Group 2, the eHHs were cultured in 17S+3UC maturation medium for four days prior to CYP3A4 induction. Finally, in Group 3, the eHHs were cultured in Takara Cellartis POWER™ Primary HEP medium for four days before CYP3A4 induction. Following three days of CYP3A4 induction, CYP3A4 activity was measured.

FIG. 29 is a bar graph showing CYP3A4 activity in eHHs, expanded hepatocytes matured in 17S+UC medium (emHH_{17S+3UC}), expanded hepatocytes maintained in Takara Cellartis POWER™ Primary HEP medium (emHH_{Takara}), or PHHs. For each cell group, CYP3A4 activity was measured in cells at baseline after culturing or upon treatment with CYP3A4 inhibitor with dimethyl sulfoxide (DMSO) or treatment with rifampicin to promote CYP3A4 activity. Single asterisk (*): p-value<0.05 for intergroup comparisons. Two asterisks (**): p-value<0.01 for intergroup comparisons. Four asterisks (****): p-value<0.0001 for intergroup comparisons. Ampersand (&): p-value<0.0001 compared to intra-group baseline. Number symbol (#): p-value<0.0001 compared to intra-group DMSO treatment. Dollar sign ($): p-value<0.05 compared to intra-group DMSO treatment.

FIG. 30 is a bar graph showing the percentage of viable hepatocytes at indicated stages of expansion, maturation, or maintenance.

FIGS. 31A-31D are a series of bar graphs showing expression levels hepatocyte biomarkers albumin (FIG. 31A), alpha-1 anti-trypsin (FIG. 31B), factor IX (FIG. 31C), and urea (FIG. 31D) that were secreted into the cell culture medium at the indicated stages of hepatocyte expansion, maturation, or maintenance.

FIG. 32 is a heatmap showing mRNA expression levels of different biomarkers for the cultured hepatocytes at the indicated stages of hepatocyte expansion, maturation, or maintenance.

FIG. 33 is a set of RNA-seq data comparing mRNA expression levels of the indicated cell identity biomarkers in PHHs, expanded and matured hepatocytes (emHHs), expanded iPSC-derived hepatocytes cultured in maturation medium (iHEPs [17S+3UC]), eHHs, and iPSC-derived hepatocytes cultured in maintenance medium (iHEPs [maint]). Shown is a heatmap (left panel) depicting the relative mRNA expression levels of mature hepatocyte markers, mesenchymal cell markers, and fetal/progenitor cell markers in the indicated cell type. Also shown are graphs (center and right panels) that depict the number of transcripts per million (TPM) of AFP or SERPINA1, which encodes A1AT, for each cell population. [17S+3UC]: maturation medium. [maint]: maintenance medium.

FIG. 34 is a set of RNA-seq data comparing mRNA expression levels of the indicated metabolic biomarkers in PHHs, emHHs, iHEPs [17S+3UC], eHHs, and iHEPs [maint]. Shown is a heatmap (left panel) depicting the relative mRNA expression levels of genes involved in the urea cycle and glutamine synthesis. Also shown are graphs (center and right panels) that depict the TPM of urea cycle genes CPS1, ARG1, aquaporin-9 (AQP9) and glutamine synthesis gene glutamate-ammonia ligase (GLUL).

FIGS. 35A-35C are bar graphs depicting the amount of secreted factor IX (FIG. 35A), A1AT (FIG. 35B), and urea (FIG. 35C) by cultured PHHs, emHHs, iHEPs [maint], and iHEPs [17S+3UC].

FIG. 36 shows RNA-seq data comparing mRNA expression levels of cytokines. A heatmap depicting relative mRNA expression levels of CXC motif chemokine ligand (CXCL) 8, CXCL5, and CXCL6 for PHHs, emHHs, iHEPs [17S+3UC], eHHs, and iHEPs [maint] is shown. Also shown is a graph that depicts the TPM of CXCL8 in the indicated cell cultures.

FIG. 37 shows micrographs of input hepatocyte and fibroblast seeds aggregated in xeno-free aggregation medium or LIFENET HEALTH® Human Hepatocyte Medium before culturing for four days in LIFENET HEALTH® Human Hepatocyte Medium supplemented with ROCK inhibitor (Ri), optionally with Pluronic F68 (P-F68) and DNase I, where indicated. Rotor speeds during culturing are indicated in rotations per minute (RPM). Also shown is a bar graph depicting the percentage of seeds recovered based as compared to input seeds prior to culturing.

FIG. 38 is a series of graphs depicting the size distribution profiles of the input hepatocyte and fibroblast seeds and the resulting seeds following four days of culturing under the indicated conditions.

FIG. 39 is a series of representative immunofluorescent micrographs staining for live and dead cells in the input seeds and the seeds following four days of culturing under the indicated conditions (scale bar: 200 μm).

FIGS. 40A and 40B are bar graphs showing the secreted amount of albumin (FIG. 40A) and factor IX (FIG. 40B) secreted in the culture medium at day 2 (D2) or day 4 (D4) of culturing under the indicated conditions.

FIG. 41 is a series of micrographs depicting hepatocyte and fibroblast seeds produced with hepatocytes that were expanded in Expand 4.0 expansion medium (left panel) or Expand 3.0 expansion medium (right panel) in two different cell lots for each condition. Scale bar is 200 μm.

FIGS. 42A and 42B are graphs showing circulating concentrations of human serum albumin (FIG. 42A) and human alpha-1 antitrypsin (FIG. 42B) in the blood of mice that were administered seeds comprising expanded human hepatocyte via intrasplenic delivery and received either constant treatment of dexamethasone or a saline control.

FIGS. 43A and 43B are pictures of a Western blot depicting immunostaining of various urea cycle proteins at post-operative day (POD) 0, 14, or 28 following the administration of seeds comprising expanded human hepatocytes in mice that received treatment with dexamethasone or a saline control (FIG. 43A) or a Ponceau-stained membrane showing the total concentration of protein loaded for immunostaining (FIG. 43B).

FIG. 44 is a set of expression data showing circulating levels of human serum albumin in mice that were administered expanded hepatocytes in the kidney capsule, in which the hepatocytes were assembled in seeds with or without normal human dermal fibroblasts (NHDFs) (top left panel) or in seeds with either NHDF or bone-marrow derived mesenchymal stromal cells (MSCs) (top right panel) at the indicated days after administration. Also shown below each panel are histology and eosin (H&E)-stained kidney tissue sections on day 28 after administration of seeds with or without NHDF (bottom left panel; scale bar: 50 μm) and kidney tissue sections showing carbamoyl phosphate synthetase expression as detected by immunohistochemical staining on day 28 after administration of seeds with MSC or NHDF (bottom right panel).

FIG. 45 is a graph comparing circulating levels of human serum albumin in mice at days 10, 20, 21 and 28 after administration of expanded hepatocytes in seeds that contained varying amounts of NHDF cells in the seed.

FIG. 46 is a set of expression data that shows circulating levels of human serum albumin in mice at the indicated days following administration of expanded human hepatocytes to the fat pad, kidney capsule, or spleen (left panels) and micrograph showing human OTC expression levels as detected by immunohistochemical staining on day 28 after administration of the hepatocytes to each location.

FIG. 47 is a schematic diagram showing the three phases of hepatocyte engraftment in a hereditary tyrosinemia model, in which phase 1, depicts hepatocyte administration to the mouse via via intrasplenic injection, phase 2 depicts cycling of administration of 2-(2-nitro-4-trifluoromethylbenzoyl)-1,3-cyclohexanedione (NTBC)-containing drinking water as the hepatocytes repopulate the liver, and phase 3 depicts the survival stage in which NTBC is completely withdrawn.

FIG. 48 is a series of graphs showing levels of circulating human albumin in mice following administration of PHHs, expanded human hepatocytes expanded in Expand 4.0 expansion medium (EHHs), matured EHHs (EHH+), control expanded hepatocytes that were cultured in a commercially available medium, or iPSC-derived hepatocytes (iHEPs).

FIG. 49 is a series of micrographs depicting immunohistochemical staining of human fumarylacetoacetate hydrolase (FAH) protein in tissue sections of livers from mice that were administered EHHs, EHHs+, control hepatocytes, or iHEPs, as described in FIG. 48. Percentages within each micrograph indicate repopulation rates, quantified from FAH-stained sections (mean±95% CI) Scale bar: 5 mm.

FIG. 50 is a graph showing the amount of human DNA in the liver of mice administered EHHs, EHHs+, control hepatocytes, or iHEPs, as detected by Alu repeats detected by real-time PCR.

FIG. 51 is a series of graphs showing plasma levels of human alpha-1 antitrypsin at the indicated time point in mice that were administered EHHs, EHHs+, control hepatocytes, or iHEPs.

FIGS. 52A and 52B are bulk RNA-sequencing (RNA-seq) data comparing gene expression of in vitro cultivated PHHs, EHHs, and EHHs+ (FIG. 52A) or the cells following four months in vivo (FIG. 52B) as shown by UMAP projections (left panels) and graphs depicting Euclidean distance to PHHs (right panels).

FIG. 53 are heatmaps showing bulk RNA-seq data in engrafted cells (PHHs, EHHs, and EHHs+) as compared to non-engrafted cells (iHEPs and control cells) for genes indicative of phase 1 or phase 2 metabolism, urea cycle proteins, or genes involved in protein synthesis. Gene expression data is shown as normalized transcript per million (TPM).

FIG. 54 is a series of micrographs depicting immunofluorescent staining of ARG1 and DAPI in liver of mice administered the indicated hepatocyte. Scale bar: 300 μm.

FIG. 55 are heatmaps showing bulk RNA-seq data in engrafted cells (PHHs, EHHs, and EHHs+) as compared to non-engrafted cells (iHEPs and control cells) for the indicated innate immune signaling genes, expressed as normalized TPM.

FIG. 56 is a Kaplan-Meier curve depicting the survival of mice administered PHHs, EHHs, EHHs+, or control cells following the complete withdrawal of NTBC.

FIG. 57 is a series of bar graphs depicting circulating levels aspartate aminotransferase (AST), alanine aminotransferase (ALT) or ammonia in mice that were administered PHHs, EHHs, EHHs+, or control cells following the complete withdrawal of NTBC.

FIG. 58 is a series of micrographs depicting staining of spleen tissue from minipigs intraarterially administered seeds comprising expanded human hepatocytes, in which staining of liver marker cytokeratin 18 (CK18) (left panel) and H&E staining (right panel) confirm presence of seeds in the spleen tissue two days after seed administration.

### DEFINITIONS

As used herein, the terms “a,” “an,” and “the” include plural referents unless the content clearly dictates otherwise. Thus, for example, reference to “a component” optionally includes a combination of two or more such components, and the like.

As used herein, the term “about,” as applied to one or more values of interest, refers to a value that falls within 10% in either direction (greater than or less than) of a stated reference value, unless otherwise stated or otherwise evident from the context (except where such number would exceed 100% of a possible value).

As used herein, the term “basal medium” refers to any cell culture medium which is appropriate for culturing human cells, including, but not limited to, Dulbecco's Modified Eagle Medium/Ham's Nutrient Mixture F-12 (DMEM/F-12), LONZA™ HCM™, William's E, HepatoZYME-SFM and any modification thereof. Modifications to the cell culture medium include various reagent additions including, but not limited to, stable forms of L-glutamine, buffering agents such as N-2-hydroxyethylpiperazine-N′-2-ethanesulfonic acid (HEPES). The basal medium may include, where desirable, antibiotic solutions, such as penicillin and/or streptomycin or gentamicin.

As used herein, the terms “comprise,” “comprising,” “comprises,” and “comprised of” are synonymous with “include,” “including,” “includes,” or “contain,” “containing,” “contains,” and are inclusive or open-ended terms that specify the presence of what follows, e.g., a component, and do not exclude or preclude the presence of additional, non-recited components, features, element, members, steps, known in the art or disclosed therein.

As used herein, the term “culturing step” refers to the process of expanding and passaging cells. This process encompasses the phase of cell culture in which the number of cells increases by cell division. When the cells have reached, e.g., 80-90% confluence, they may be passaged and seeded onto additional cell culture surfaces. For example, one passage of PHHs may require at least 3 days to reach confluence. Cells can be continuously passaged and cultured for 120 days. Alternatively, cells can be passaged until they become transformed or lose hepatic phenotype. A culturing step may include a step of initially seeding and expanding the cells, which may be referred to as step P0, and then passaging the cells in one or more sequential passages (e.g., step P1, step P2, step P3, and onward until the cells become transformed or lose hepatic phenotype).

As used herein, the terms “decrease,” “reduce,” and related variations refer to a lesser amount or a lesser degree. In some embodiments, the terms “decrease” or “reduce” can mean a decrease by at least 10% as compared to a reference level (e.g., the absence of a given treatment) and can include, for example, a decrease by 10% or more (e.g., by 10%, 15%, 20%, 25%, 30%, 35%, 40%, 45%, 50%, 55%, 60%, 65%, 70%, 75%, 80%, 85%, 90%, 95%, or 100%) as compared to a reference level.

As used herein, the terms “expand,” “expands,” “expanding,” and “expansion” refer to an increase in the number of what follows, e.g., a population of hepatocytes (e.g., PHHs). An “expansion step” refers to a phase of cell culture in which the number of hepatocytes increases by cell division. As used herein in the context of a plurality of agents that together or collectively “expand” a population of hepatocytes, describes instances in which each agent, individually, may or may not achieve the indicated function, but when the agents are combined, the indicated expansion is achieved.

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. In the context of a gene that encodes a protein product, the terms “gene expression” and the like are used interchangeably with the terms “protein expression” and the like. Expression of a gene or protein of interest in a patient can manifest, for example, by detecting: an increase in the quantity or concentration of mRNA encoding corresponding protein (as assessed, e.g., using RNA detection procedures described herein or known in the art, such as reverse transcription quantitative polymerase chain reaction (RT-qPCR) and RNA seq techniques), an increase in the quantity or concentration of the corresponding protein (as assessed, e.g., using protein detection methods described herein or known in the art, such as enzyme-linked immunosorbent assays (ELISA), among others), and/or an increase in the activity of the corresponding protein (e.g., in the case of an enzyme, as assessed using an enzymatic activity assay described herein or known in the art) in a sample obtained from the patient. As used herein, a cell is considered to “express” a gene or protein of interest if one or more, or all, of the above events can be detected in the cell or in a medium in which the cell resides. For example, a gene or protein of interest is considered to be “expressed” by a cell or population of cells if one can detect (i) production of a corresponding RNA transcript, such as an mRNA template, by the cell or population of cells (e.g., using RNA detection procedures described herein); (ii) processing of the RNA transcript (e.g., splicing, editing, 5′ cap formation, and/or 3′ end processing, such as using RNA detection procedures described herein); (iii) translation of the RNA template into a protein product (e.g., using protein detection procedures described herein); and/or (iv) post-translational modification of the protein product (e.g., using protein detection procedures described herein).

The terms “expression level” or “level of expression” in general are used interchangeably and generally refer to the amount of a marker in a biological sample. “Expression” generally refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., post-translational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

The terms “increase,” “enhance,” and related variations are all used herein to mean an elevated amount or elevated degree. In some embodiments, the terms “increase” or “enhance,” can mean an increase of 10% or more (e.g., by 10%, 15%, 20%, 25%, 30%, 35%, 40%, 45%, 50%, 55%, 60%, 65%, 70%, 75%, 80%, 85%, 90%, 95%, 100%, or more than 100%) as compared to a reference level. In some embodiments, the terms “increase” or “enhance” can refer to an increase of about 2-fold, or about 3-fold, about 4-fold, about 5-fold, about 10-fold, or more, as compared to a reference level.

As used herein, the term “inhibitor” refers to any compound, natural or synthetic, which can reduce the activity of a target protein or signaling pathway. An inhibitor may attenuate or prevent the activity of a target protein either directly or indirectly. Direct inhibition can be obtained, for instance, by binding to a protein and reducing or eliminating interaction of the protein and an endogenous molecule, such as an enzyme, a substrate, or other binding partner, thereby diminishing the activity of the protein. For instance, an inhibitor may bind an enzyme active site and sterically preclude binding of an endogenous substrate at this location, thus decreasing the enzymatic activity of the protein. Alternatively, indirect inhibition can be obtained, for instance, by binding to a protein that promotes the activity of a target protein by inducing a conformational change or catalyzing a chemical modification of the target protein. For instance, indirect inhibition of a target protein may be achieved by binding and inactivating a kinase that catalyzes the phosphorylation of, and thus activates, the target protein.

As used herein, the terms “biomarker” or “marker” are used interchangeably herein to refer to a DNA, RNA, protein, carbohydrate, or glycolipid-based molecular marker, the expression or presence of which can be detected by standard methods in the art (or methods disclosed herein) in a sample (e.g., a cell sample, a cell population sample, a culture medium sample, or a tissue sample). Expression of such a marker may be determined to be higher or lower in a population of hepatocytes (e.g., PHHs) that have been expanded and/or matured using the disclosed compositions and/or according to the disclosed methods, as compared to a population of hepatocytes that were not expanded or matured with said compositions or methods.

As used herein, the term “maturing” or any variation thereof refers to the process of developing cells into a population that is distinct from a progenitor population. For example, a population of hepatocytes that has undergone a maturing step as disclosed herein has a mature hepatocyte phenotype, which may be evaluated by gene expression analysis (e.g., an upregulation of transcripts associated with mature hepatocytes and downregulation of transcripts associated with progenitors or cholangiocytes) and/or hepatocyte functional assays (e.g., urea secretion, CYP3A4 activity).

As used herein, the term “maturation supplement” refers to a reagent or additive that may be included in a basal cell culture medium for promoting maturation of a cell or a population of cells (e.g., hepatocytes).

As used herein, the terms “one or more” or “a combination thereof,” in reference to one or more member(s) of a group, is clear per se, by means of further exemplification, the term encompasses inter alia a reference to any one of said members, or to any two or more of said members, e.g., ≥3, ≥4, ≥5, ≥6, or ≥7, etc., of said members, and up to all said members.

“Primary cells,” and “primary cultures” are used interchangeably herein to refer to cells and cell cultures that have been harvested from human livers and cryogenically stored immediately without being cultured in vitro for any amount of time. Cells can be harvested from an individual by any convenient method such as biopsy or isolation from whole donated livers. An appropriate solution can be used for dispersion or suspension of the harvested cells. The cells can be used immediately, or they can be stored, frozen, for long periods of time, and then later thawed and reused.

As used herein, the term “primary human hepatocytes” (or “PHHs”) refer to major parenchymal cells in the liver. Specifically, these cells are of human origin and have the capacity to replicate and increase cell number in response to liver injury. It is known in the art that such cells express one or more gene selected from hepatocyte nuclear factor 4-alpha (HNFA), albumin (ALB), and a member of the cytochrome P450 (CYP) gene family (e.g., CYP 1, CYP2, CYP3, CYP4, CYP5, CYP7, CYP8, CYP11, CYP17, CYP19, CYP20, CYP21, CYP24, CYP26, CYP27, CYP39, CYP46, CYP51, or a combination thereof). It is also known that such cells do not express the alpha-fetoprotein (AFP) gene. It is additionally known in the art that such cells express biomarkers such as HNF4a, albumin, A1AT, and transferrin, and also secrete urea.

As used herein, the term “subject” refers to any organism to which a composition in accordance with the invention may be administered, e.g., for experimental, diagnostic, prophylactic, and/or therapeutic purposes. A subject may be any animal (e.g., a mammal). A subject may be in need of treatment and/or may receive treatment by administration of a population of hepatocytes (e.g., a population of hepatocytes prepared by the methods disclosed herein). In preferred embodiments, the subject is a human.

As used herein, the term “serum replacement component” refers to a reagent that may be added to cell culture medium that does not include serum (i.e., serum-free medium). A serum replacement component may be a specific formulation that includes, e.g., amino acids, antioxidants, transferrin or transferrin substitutes, insulin or insulin substitutes, and lipids.

As used herein, the term “three-dimensional (3D) culture system” refers to a format for in vitro cultivation of cells in which a population of cells are embedded in a 3D matrix during culturing. A 3D matrix for a 3D culture system may include a hydrophilic polymer such as an ECM protein (e.g., a collagen, a laminin, entactin, a fibronectin, a vitronectin, and/or perlecan), a glycosaminoglycan (e.g., hyaluronic acid), alginate, a synthetic polymer (e.g., polyglycolic acid, polylactic acid, polyorthoester, and/or polycaprolactone), or a combination thereof that maintains cell-cell and cell-polymer contacts in an aggregated cell cluster. A 3D culture system may include a hydrophilic gel (i.e., a hydrogel) that comprises a hydrophilic polymer to maintain cell-cell and cell-polymer contacts in an aggregated cell cluster. A hydrogel may be a commercially available hydrogel such as MATRIGEL™ or CULTREX™. In some embodiments, a 3D culture system includes a suspension cell culture in which a population of cells are dispersed in a liquid medium under continuous shaking or rotation in a cell culture vessel (e.g., a spinner flask, a stir flask, or a bioreactor).

As used herein, the term “two-dimensional (2D) culture system” refers to a format for in vitro cultivation of cells in which a population of cells are cultured to grow on a surface (e.g., a surface of a cell culture vessel such as a plate, a flask, a cell stack, a microcarrier, or a microwell). In some embodiments, all or substantially all of the cells contact the surface. In some embodiments, a surface a cell culture vessel is coated in a polymer such as an ECM protein (e.g., a collagen, a laminin, entactin, a fibronectin, a vitronectin, and/or perlecan), a glycosaminoglycan (e.g., hyaluronic acid), a synthetic polymer (e.g., polyglycolic acid, polylactic acid, polyorthoester, and/or polycaprolactone), or a combination thereof such that the basal surface of a cell contacts the surface of the cell culture vessel (e.g., via adhesion of basal cell surface receptors to the surface of the cell culture vessel). In some embodiments, the surface of the cell culture vessel is a flat surface. In some embodiments, the surface of the cell culture vessel is a curved surface (e.g., the surface has a convex surface or a concave surface).

As used herein, the term “xeno-free” refers to cell culture components or reagents that do not contain any unrefined or raw materials (e.g., a serum or tissue extract) that originate from a species that is different from the cell in culture. For example, a xeno-free cell culture medium for the cultivation of human cells would does not contain serum or tissue extracts from any non-human animals but may contain serum or tissue extracts that originate from a human (e.g., human platelet lysate). Xeno-free cell culture reagents may include recombinant proteins.

### DETAILED DESCRIPTION

The present disclosure provides compositions and methods that can be used for the two-dimensional or three-dimensional expansion and/or maturation of hepatocytes (e.g., primary human hepatocytes (PHHs) or iPSC-derived hepatocytes). In accordance with the compositions and methods described herein, a subject (e.g., a human) may be implanted with a population of the expanded hepatocytes. The disclosed methods for expanding hepatocytes result in robust and widely applicable culture expansion. Furthermore, these methods are not limited by the source of the hepatocytes or restrictive age limits on donor PHHs, thereby expanding the pool of eligible PHH donors and feasibly reducing the shortage of donor PHHs.

This invention is based, at least in part, on the discovery of defined culture conditions, including a basal medium for human cells, a fibroblast growth factor (FGF), an epidermal growth factor (EGF), an R-spondin, and a transforming growth factor-beta (TGF-beta) inhibitor, that allow hepatocytes, such as PHHs or hepatocytes obtained from reprogrammed cells (e.g., iPSC-derived hepatocytes), to be cultured for an increased number of passages as compared to existing cell culture medium formulations. Furthermore, the defined culture conditions also reflect the discovery of a reduced set of components required for expansion and/or maturation to reduce costs as compared to media with more or more expensive components. The defined culture conditions may include the addition of one or more additives or supplements, which are described in more detail herein. The completely defined, xeno-free culture conditions also result in large-scale, robust, expansion of hepatocytes (e.g., PHHs) that advantageously allows more cells to be obtained from a single cell or from a collection of cells as starting material than was possible using previous methods. These advantages allow for increasing the availability of cell sources or donor cells for therapeutic purposes (e.g., to supplement or rescue native liver function).

The present disclosure also provides compositions and methods that can be used for the maturation of hepatocytes, including hepatocytes that were expanded in an expansion medium described herein. This maturation of hepatocytes includes culturing the hepatocytes (e.g., PHHs or iPSC-derived hepatocytes, including expanded PHHs or expanded iPSC-derived hepatocytes) in a basal medium for human cells with one or more hepatocyte maturation supplements to reduce the rate of cell proliferation and/or reduce the likelihood of cultivating cells with a progenitor phenotype, thereby cultivating the hepatocytes such that they are matured into a distinct population. In accordance with the compositions and methods described herein, a subject (e.g., a human) may be implanted with a population of matured hepatocytes. The disclosed methods for maturing hepatocytes (e.g., PHHs or iPSC-derived hepatocytes) result in robust and widely applicable cell maturation.

### A. Expansion of Hepatocytes

The compositions and methods described herein include expansion of hepatocytes. In some embodiments of the compositions and methods described herein, the hepatocytes are primary human hepatocytes (PHHs). In some embodiments of the compositions and methods described herein, the hepatocytes are iPSC-derived hepatocytes. In some embodiments, culturing the hepatocytes (e.g., PHHs or iPSC-derived hepatocytes) in an expansion method disclosed herein allows the cells to multiply and retain their hepatocyte identity. In some embodiments, populations of expanded hepatocytes are formed using the expansion method include hepatic stem cells or progenitor-like cells or iPSCs. In some embodiments, the method further includes maturing a population of expanded hepatocytes (e.g., via the maturation methods described herein).

In some embodiments, the hepatocytes are PHHs that are obtained from mature tissue (e.g., mature liver tissue). A PHH or a population of PHHs may be obtained by any suitable method. In some embodiments, cells are isolated by collagenase digestion, for example, as described in Dorell et al. Hepatology. 48:1282-91, 2008, which is hereby incorporated by reference. In some embodiments, collagenase digestion is performed on a tissue biopsy. In some embodiments, collagenase and accutase digestion are used to obtain the PHHs.

PHHs are present in the liver. In some embodiments, the method includes culturing a fragment of tissue which includes liver epithelium. In some embodiments, the PHHs are isolated from a tissue fragment. For example, in the context of liver, the tissue fragment may include a portal vein, a liver biliary duct, or biliary duct tissue. Liver PHHs can be isolated from normal liver tissues using FACS-based sorting to exclude EpCAM⁺ progenitor cells such that the population of PHHs after processing does not include a detectable amount of EpCAM⁺ progenitor cells. In some embodiments, PHHs isolated from normal liver tissue may contain EpCAM+ progenitor cells such that the level of EpCAM+ progenitor cells is less than 10% (e.g., less than 10%, less than 9%, less than 8%, less than 7%, less than 6%, less than 5%, less than 4%, less than 3%, less than 2%, or less than 1%).

In some embodiments, the hepatocytes are derived from reprogrammed cells. In some embodiments, a reprogrammed cell may be a stem cell (e.g., a pluripotent stem cell) that was differentiated to have hepatocyte identity. In some embodiments, the hepatocytes are obtained from a population of differentiated stem cells such as iPSCs. Hepatocytes that derive from iPSCs are commercially available. Exemplary iPSC-derived hepatocytes are ICELL® Hepatocytes 2.0 (FUJIFILM Cellular Dynamics International, Inc.).

Hepatocytes (e.g., PHHs or hepatocytes derived from a population of reprogrammed cells such as a population of iPSCs) are terminally differentiated and are not progenitor cells (e.g., hepatocyte-like cells, liver-derived progenitor cells, or EpCAM⁺ progenitor cells), cannot be differentiated into a different cell type, and do not require further co-culturing with an additional cell type to adopt a hepatocyte cellular fate. Without being bound by theory, the terminally differentiated hepatocytes are capable of proliferation upon culturing in a cell culture method described herein due to signaling induced by environmental cues (e.g., a cell culture medium and/or a surface of a cell culture vessel).

In some embodiments, a single cell is isolated and cultured in a cell culture medium. In other embodiments, a population of cells is isolated and cultured in a cell culture medium.

In some embodiments, the cells may be cultured after isolation for about 5 days, about 10 days, about 15 days, about 20 days, about 25 days, or about 30 days. In some embodiments, the cells may be cultured after isolation for at least about 5 days. In some embodiments, the cells may be cultured after isolation for at least 10 days. In some embodiments, the cells may be cultured after isolation for at least 15 days. In some embodiments, the cells may be cultured after isolation for at least 20 days. In some embodiments, the cells may be cultured after isolation for at least 25 days. In some embodiments, the cells may be cultured after isolation for at least about 30 days.

The length of time the cells are cultured may depend on the recovery of the cells after isolation. For instance, the cells may be cultured until they reach about 80%, about 85%, or about 90% confluence in a cell culture vessel (e.g., a microwell plate, a flask, or a Petri dish). In certain aspects, the cells are expanded in culture longer to improve the homogeneity of the cell phenotype in the cell population or to stabilize the cell state of expanded cells.

In some embodiments, provided is a method for obtaining a population of expanded hepatocytes including culturing hepatocytes in an expansion medium using the method as described herein.

In some embodiments, following isolation and subsequent culturing, the cells (e.g., hepatocytes; e.g., PHHs or iPSC-derived hepatocytes) are prepared for storage and are frozen for later use. In other embodiments, following isolation and subsequent culturing, the cells (e.g., hepatocytes, e.g., PHHs or iPSC-derived hepatocytes) are expanded into a larger population via methods of cell cultivation described herein.

In some embodiments, the method includes culturing the hepatocytes or obtaining the population of expanded hepatocytes from a single cell. Advantageously, this allows a homogenous population of cells to form. In some embodiments, the method includes culturing the cells in an expansion medium for a duration of 10 to 50 days (e.g., 10 to 50 days, 10 to 45 days, 10 to 40 days, 10 to 35 days, or 10 to 30 days; e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days).

In some embodiments, the culturing step includes expanding plated cells (step P0) and a first passage of expanded cells (step P1). In some embodiments, the culturing step further includes a second passage (step P2), a third passage (step P3), a fourth passage (step P4), a fifth passage (step P5), a sixth passage (step P6), or more than six passages (step P6+n). A passage may have a duration of 5 to 25 days (e.g., 5 to 7 days, 5 to 10 days, 5 to 14 days, 5 to 20 days, 5 to 25 days, 7 to 10 days, 7 to 14 days, 10 to 15 days, 10 to 20 days, 10 to 25 days, 14 to 20 days, 14 to 25 days, or 20 to 25 days; e.g., 5, 6, 7, 8, 9, 10, 11, 12, 13, 14, 15, 16, 17, 18, 19, 20, 21, 22, 23, 24, 25 days). The duration of each passage may depend on the doubling time of the population of cells, the surface area of the cell culture vessel, the length of time required for the cell population to reach a desired cell density (e.g., 70% to 90% confluency), or a combination thereof. Furthermore, the number of passages may depend on cell morphology, cell viability, and/or proliferative state of the cell population. For instance, a population of hepatocytes may be passaged as long as the cells divide at a consistent rate or maintain hepatocyte identity and cell fate (e.g., the cells do not exhibit mesenchymal biomarkers or morphology) as measured by one or more hepatocyte biomarkers described herein.

In some embodiments, step P0 has a duration of 5 to 25 days, e.g., 7 to 20 days (e.g., 5, 6, 7, 8, 9, 10, 11, 12, 13, 14, 15, 16, 17, 18, 19, 20, 21, 22, 23, 24, or 25 days). In some embodiments, step P0 has a duration of longer than 25 days (e.g., about 30 days). In some embodiments, step P0 has a duration of about 10 to about 16 days (e.g., about 10, about 11, about 12, about 13, about 14, about 15, or about 16 days). In some embodiments, step P0 has a duration of about 10 to about 14 days (e.g., about 10, about 11, about 12, about 13, about 14 days). In some embodiments, step P0 has a duration of about 14 days.

In some embodiments, step P1 has a duration of 5 to 25 days, e.g., 7 to 20 days (e.g., 5, 6, 7, 8, 9, 10, 11, 12, 13, 14, 15, 16, 17, 18, 19, 20, 21, 22, 23, 24, or 25 days). In some embodiments, step P1 has a duration of longer than 25 days (e.g., about 30 days). In some embodiments, step P1 has a duration of 10 to 16 days (e.g., about 10, about 11, about 12, about 13, about 14, about 15, or about 16 days). In some embodiments, step P0 has a duration of 10 to 14 days (e.g., about 10, about 11, about 12, about 13, about 14 days). In some embodiments, step P1 has a duration of about 14 days.

In some embodiments, step P0 and/or step P1 includes seeding the hepatocytes at a density of 200 viable cells/cm² to 14,000 viable cells/cm², e.g., 200 to 1,000 viable cells/cm² (e.g., about 200, about 300, about 400, about 500, about 600, about 700, about 800, about 900, or about 1,000 viable cells/cm²), 1,000 viable cells/cm² to 10,000 viable cells/cm² (e.g., about 1,000, about 2,000, about 3,000, about 4,000, about 5,000, about 6,000, about 7,000, about 8,000, about 9,000, or about 10,000 viable cells/cm²), or 10,000 viable cells/cm² to 14,000 viable cells/cm² (e.g., about 10,000, about 11,000, about 12,000, about 13,000, or about 14,000 viable cells/cm²). In some embodiments, step P0 includes seeding the hepatocytes at a density of about 600, about 625, about 650, about 675, about 700, about 725, about 750, about 775, or about 800 viable cells/cm². In some embodiments, step P1 includes seeding the hepatocytes at a density of about 900, about 925, about 1,000, about 1,025, about 1,050, about 1,075, or about 1,110 viable cells/cm².

In some embodiments, the population of hepatocytes (e.g., PHHs) expand by at least 10-fold, at least 20-fold, at least 30-fold, at least 40-fold, at least 50-fold, at least 60-fold, at least 70-fold, at least 80-fold, at least 90-fold, at least 100-fold, at least 110-fold, at least 120-fold, at least 130-fold, at least 140-fold, or at least 150-fold during step P0. In some embodiments, the population of hepatocytes (e.g., PHHs) expand by 10-fold to 200-fold during step P0 (e.g., about 10-fold, about 15-fold, about 20-fold, about 25-fold, about 30-fold, about 35-fold, about 40-fold, about 45-fold, about 50-fold, about 60-fold, about 70-fold, about 80-fold, about 90-fold, about 100-fold, about 110-fold, about 120-fold, about 130-fold, about 140-fold, about 150-fold, about 160-fold, about 170-fold, about 180-fold, about 190-fold, or about 200-fold). For example, upon seeding hepatocytes at a density of 200 to 1,000 viable cells/cm² during the initial passage (i.e., step P0), the cell yield following expansion of hepatocytes during step P0 may be 4×10³ viable cells/cm² to 1.5×10⁵ viable cells/cm². In preferred embodiments, the expanded hepatocytes (e.g. PHHs) are further passaged (i.e., step P1) to further expand the population of hepatocytes. The hepatocytes may be passaged after reaching about 70% (e.g., about 70%, about 75%, about 80%, about 85%, about 90%, or about 95%) confluence during step P0 of the cell culturing method.

In some embodiments, the population of hepatocytes (e.g., PHHs) expand by about 500-fold, about 600-fold, about 700-fold, about 800-fold, about 900-fold, about 1,000-fold, about 1,100-fold, about 1,200-fold, about 1,300-fold, about 1,400-fold, about 1,500-fold, about 1,600-fold, about 1,700-fold, about 1,800-fold, about 1,900-fold, about 2,000-fold, about 2, 100-fold, about 2,200-fold, about 2,300-fold, about 2,400-fold, about 2,500-fold, about 2,600-fold, about 2,700-fold, about 2,800-fold, about 2,900-fold, about 3,000-fold, about 3,100-fold, about 3,200-fold, about 3,300-fold, about 3,400-fold, about 3,500-fold, about 3,600-fold, about 3,700-fold, about 3,800-fold, about 3,900-fold, about 4,000-fold, about 4, 100-fold, about 4,200-fold, about 4,300-fold, about 4,400-fold, about 4,500-fold, about 4,600-fold, about 4,700-fold, about 4,800-fold, about 4,900-fold, about 5,000-fold, about 5, 100-fold, about 5,200-fold, about 5,300-fold, about 5,400-fold, about 5,500-fold, about 5,600-fold, about 5,700-fold, about 5,800-fold, about 5,900-fold, about 6,000-fold, about 6, 100-fold, about 6,200-fold, about 6,300-fold, about 6,400-fold, about 6,500-fold, about 6,600-fold, about 6,700-fold, about 6,800-fold, about 6,900-fold, about 7,000-fold, 7,100-fold, about 7,200-fold, about 7,300-fold, about 7,400-fold, about 7,500-fold, about 7,600-fold, about 7,700-fold, about 7,800-fold, about 7,900-fold, about 8,000-fold, about 8,100-fold, about 8,200-fold, about 8,300-fold, about 8,400-fold, about 8,500-fold, about 8,600-fold, about 8,700-fold, about 8,800-fold, about 8,900-fold, about 9,000-fold, about 9,100-fold, about 9,200-fold, about 9,300-fold, about 9,400-fold, about 9,500-fold, about 9,600-fold, about 9,700-fold, about 9,800-fold, about 9,900-fold, or about 10,000-fold cumulatively in step P0 and step P1.

Achieving high cell yields of hepatocytes following expansion (e.g., expansion of PHHs or iPSC-derived hepatocytes) is critical for therapeutic applications in which hepatocytes may be administered to, engrafted in, or implanted in a subject in need thereof (e.g., a subject that has liver dysfunction or liver failure or a subject that is at risk of liver dysfunction or liver failure) since the availability of PHHs for transplantation is limited and therefore poses a significant problem. A mouse transplant, for example, requires a minimum of 1×10⁵ hepatocytes for successful engraftment. Approximately 1×10⁷ hepatocytes may be required for a successful engraftment in a human subject.

In some embodiments, the hepatocytes (e.g., PHHs or iPSC-derived hepatocytes) are cultured in the presence of 5% carbon dioxide. In some embodiments, the hepatocytes (e.g., PHHs or iPSC-derived hepatocytes) are cultured at a temperature of 37° C. In some embodiments, PHHs are cultured in hypoxic conditions, in which atmospheric oxygen level is below 20.9% or a hypoxic mimetic (e.g., a hypoxic mimetic described herein).

In some embodiments, the cells will expand at a rate of more than two (e.g., 3, 4, 5, 6, 7, 8, 9, 10, 11, 12, 13, 14, 15, 16, 17, 18, 19, 20, 21, 22, 23, 24, 25, 26, 27, 28, 29, or 30) population doublings a week.

In some embodiments, the method includes replacing the culture medium one or more times during a method of culturing. For example, the culture medium may be replaced with fresh medium during culturing because one or more components of the culture medium (e.g., one or more components added to the culture medium) degrade or are metabolized by the cells during culturing. In some embodiments, the culture medium is replaced about every 16 hours, about every 24 hours, about every 36 hours, about every 48 hours, about every 60 hours, or about every 72 hours. In some embodiments, the culture medium is replaced every 16 to 24 hours, every 24 to 48 hours, every 24 to 60 hours, or every 24 to 72 hours. In some embodiments, the culture medium is replaced every 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 hours.

The expansion medium may induce or promote the survival and/or proliferation of cells for at least 7 days of culture (e.g., at least 7, at least 10, at least 15, at least 20, at least 30, at least 50, or at least 100). In some embodiments, the population of expanded hepatocytes includes 70% or more viable cells (e.g., 70%, 75%, 80%, 85%, 90%, 91%, 92%, 93%, 94%, 95%, 96%, 97%, 98%, or 99%). Cellular viability may be assessed using any viability determination method known in the art such as, e.g., Hoechst staining, trypan blue staining, propidium iodide staining and measuring by any suitable quantitative method including microscopy (e.g., immunofluorescent microscopy) or flow cytometry. Proliferation can be evaluated using techniques known in the art, such as bromo-2′-deoxyuridine (BrdU) staining, 5-ethynyl-2′-deoxyuridine (Edu) staining, Ki67 staining, or calculating cell growth via a growth curve.

In some embodiments, following the culturing step, the expression profile of the hepatocytes (e.g., PHHs or iPSC-derived hepatocytes) includes expression of Ki67 (e.g., protein or mRNA transcript expression levels) by about 15% or more hepatocytes in the population (e.g., at least 15%, 20%, 25%, 30%, 35%, 40%, 45%, 50%, 55%, 60%, 65%, 70%, 75%, or more). In some embodiments, following the culturing step, the expression profile of the PHHs includes expression of Ki67 by at most 15% (e.g., at most 10%, 5%, 4%, 3%, 2%, or 1%) of the PHHs.

Following culturing, the method may further include obtaining and/or isolating one or more cultured hepatocytes. For example, following cultivation of a population of hepatocytes (e.g., PHHs or iPSC-derived hepatocytes), it may be useful to remove one or more hepatocytes cultured in the cell culture medium (e.g., an expansion medium or a maturation medium described herein) for use in subsequent applications. For example, it may be useful to isolate a single hepatocyte following hepatocyte expansion in an expansion medium. Alternatively, it may be useful to obtain a population of hepatocytes following expansion in an expansion medium.

In some embodiments, following hepatocyte expansion, the expanded hepatocyte population may undergo maturation in the presence of a maturation medium including a basal medium for human cells to which is added one or more hepatocyte maturation supplements. In some embodiments, the maturation step immediately follows hepatocyte expansion. In some embodiments, the maturation step does not immediately follow hepatocyte expansion.

The cells produced by the methods described herein can be used immediately. Alternatively, the cells can be frozen in liquid nitrogen and stored for long periods of time for thawing and culturing later. For example, cells can be frozen in a culture medium that includes a cryoprotective agent, such as a medium that includes 10% dimethylsulfoxide (DMSO), 50% serum, 40% buffered medium, or one or more other agents known in the art.

In some embodiments, following culturing in an expansion medium, the expanded hepatocytes (e.g., PHHs or iPSC-derived hepatocytes) are prepared for cryopreservation. For example, a population of expanded hepatocytes may be prepared for cryopreservation by dissociating expanded population of hepatocyte cultures and mixing them with a freezing medium that includes a cryoprotective agent such as Recovery cell culture freezing medium (Gibco) or CRYOSTOR® (Biolife Solutions) and freezing following standard procedures. One or more aliquots of expanded hepatocytes may be cryopreserved for at least a week, at least a month, at least a year, or longer. One or more aliquots of frozen cells may be recovered after cryopreservation by thawing the frozen expanded hepatocytes, embedding the thawed hepatocytes in an extracellular matrix (ECM) (e.g., a laminin, a collagen, a vitronectin, a fibronectin, or a combination thereof) and culturing the expanded hepatocytes in an expansion medium of the invention.

In some embodiments, the culture medium may be supplemented with a rho kinase (ROCK) inhibitor, such as Y-27632 (CAS No. 129830038-2), following thawing. In some embodiments, the culture medium may be supplemented with about 10 μM Y-27632 after thawing. In some embodiments, the culture medium is supplemented with Y-27632 for the first 1, 2, 3, 4, 5 or less days after thawing, preferably for the first 3 or 4 days. In some embodiments, Y-27632 is not present in the culture medium after the first 3, 4, 5, 6 or more days, preferably after the first 3 or 4 days. This thawing method may be used for expansion of hepatocytes. In some embodiments, a ROCK inhibitor such as Y-27632 is not added to the culture medium after thawing.

### B. Expansion Medium

An expansion medium of the disclosure is composed of a basal cell culture medium to which one or more proteins and/or supplements are added. The expansion medium may be used for cultivating hepatocytes (e.g., PHHs or iPSC-derived hepatocytes) as described herein. Components of an expansion medium are described below.

i. Basal Cell Culture Medium

The expansion medium may include a basal cell culture medium such as a commercially available basal cell culture medium that is suitable for the in vitro cultivation of mammalian (e.g., human) cells. In some embodiments, the basal cell culture medium is Takara Cellartis POWER™ Primary HEP medium. In some embodiments, the basal cell culture medium is Lonza HCM™ cell culture medium. In some embodiments, the basal cell culture medium is William's E medium. In some embodiments, the basal cell culture medium is LIFENET HEALTH® Human Hepatocyte Media.

ii. Growth Factors and Signaling Pathway Agonists

The expansion medium may include one or more different types of growth factors, such as a fibroblast growth factors (FGFs), an epidermal growth factor (EGF), a hepatocyte growth factor (HGF), or a combination thereof. In some embodiments, an FGF, an EGF, and an HGF are added to the basal cell culture medium. In some embodiments, the expansion medium includes an FGF or a fragment thereof (e.g., a fragment that retains binding to a fibroblast growth factor receptor (FGFR)). In some embodiments, the expansion medium includes an EGF or a fragment thereof (e.g., a fragment that retains binding to an epidermal growth factor receptor (EGFR)).

FGF family members possess broad mitogenic and cell survival activities, and are involved in a variety of biological processes, including embryonic development, cell growth, morphogenesis, tissue repair, tumor growth and invasion. FGFs stimulate cells by interacting with cell surface FGFR. Four closely related receptors (i.e., FGFR1, FGFR2, FGFR3, and FGFR4) have been identified. Most FGFs bind more than one receptor (Ornitz J. Biol. Chem. 273(9):5349-57, 1998). However, FGF10 and FGF7 are unique among FGFs in that they interact only with a specific isoform of FGFR2, designated FGFR2b, which is expressed exclusively by epithelial cells (Igarashi, J. Biol. Chem. 273(21):13230-5, 1998).

In some embodiments, the FGF is a human FGF. In some embodiments, the FGF is a recombinant FGF. In some embodiments, the FGF added to the expansion medium is selected from the group consisting of FGF1, FGF2, FGF3, FGF4, FGF5, FGF6, FGF7, FGF8, FGF9, FGF10, FGF16, FGF17, FGF18, FGF19, FGF20, FGF21, FGF22, FGF23, or a combination thereof. In some embodiments, only one FGF is added to the expansion medium. In some embodiments, the FGF is FGF10. In some embodiments, the FGF that is added to the expansion medium includes FGF10 and one or more FGFs (e.g., 1, 2, 3, or more than 3 FGFs). In preferred embodiments, the FGF added to the expansion medium does not include FGF7, which is also known in the art as keratinocyte growth factor (KGF). In some embodiments, FGF10 is added to the expansion medium. In some embodiments, FGF10 and FGF3 and/or FGF22 are added to the expansion medium. In some embodiments, FGF7 is not added to the expansion medium. In some embodiments, one or more FGFs are each added to the expansion medium at a concentration of 5 ng/ml to 500 ng/ml (e.g., 5 to 25 ng/ml, 5 to 50 ng/ml, 10 to 25 ng/ml, 10 to 50 ng/ml, 20 to 50 ng/ml, 25 to 100 ng/mL, 50 to 100 ng/mL, 50 to 150 ng/ml, 50 to 500 ng/ml, 100 to 200 ng/ml, 200 to 300 ng/ml, 300 to 400 ng/ml, or 400 to 500 ng/ml; e.g., 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, or 500 ng/ml).

EGF is a protein that stimulates cell growth and differentiation by binding to its receptor, EGFR. In some embodiments, any suitable EGF may be used. In some embodiments, the EGF includes a human EGF. In some embodiments, the EGF includes a recombinant EGF (e.g., a recombinant human EGF).

In some embodiments, the an EGF is added to the expansion medium at a concentration of 5 ng/ml to 500 ng/mL (e.g., 5 to 50 ng/ml, 10 to 50 ng/ml, 10 to 100 ng/ml, 10 to 100 ng/ml, 25 to 50 ng/ml, 25 to 100 ng/ml, 50 to 150 ng/ml, 100 to 200 ng/ml, 200 to 300 ng/ml, 300 to 400 ng/ml, or 400 to 500 ng/ml; e.g., 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, or 500 ng/ml). In some embodiments, EGF is substituted with an alternative compound that activates EGFR. For example, an insulin-like growth factor (IGF) may be substituted for EGF. In some embodiments, the expansion medium does not include EGF.

In some embodiments, the expansion medium includes an HGF. In some embodiments, an HGF is added to the expansion medium. Hepatocyte growth factor/scatter factor (HGF/SF) is a morphogenic factor that regulates cell growth, cell motility, and morphogenesis by activating a tyrosine kinase signaling cascade after binding to the proto-oncogenic c-Met receptor. In some embodiments, the HGF is human HGF. In some embodiments, the HGF is a recombinant HGF. In some embodiments, an HGF is added to the expansion medium at a concentration of 1 ng/ml to 200 ng/ml (e.g., 1 to 20 ng/ml, 10 to 30 ng/ml, 10 to 50 ng/ml, 10 to 100 ng/ml, 20 to 50 ng/ml, 20 to 100 ng/ml, 50 to 75 ng/ml, 60 to 80 ng/ml, 80 to 100 ng/ml, 100 to 125 ng/ml, 125 to 150 ng/ml, 150 to 175 ng/ml, or 175 to 200 ng/ml; e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95, 100, 110, 120, 130, 140, 150, 160, 170, 180, 190, or 200 ng/ml). In some embodiments, HGF is substituted with a compound that activates the HGF receptor, such as oligopeptide N-hexanoid-Tyr-Ile-(6)aminohexanoic amide, which is also known in the art as dihexa (CAS No. 1401708-83-5).

In some embodiments, the expansion medium further comprises a transforming growth factor (TGF), a polypeptide growth factor. The TGF may be TGF-alpha, TGF-beta-1, TGF-beta-22, TGF-beta-3, or a combination thereof. In some embodiments, the TGF is a human TGF. In some embodiments, the TGF is a recombinant TGF. In some embodiments, a TGF is added to the expansion medium at a concentration of 2 ng/ml to 50 ng/ml (e.g., 2 to 10 ng/ml, 5 to 20 ng/ml, or 20 to 50 ng/mL; e.g., 2, 3, 4, 5, 6, 7, 8, 9, 10, 11, 12, 13, 14, 15, 16, 17, 18, 19, 20, 25, 30, 35, 40, 45, or 50 ng/ml). In preferred embodiments, the expansion medium does not include TGF-alpha.

In some embodiments, during culturing of hepatocytes (e.g., PHHs or iPSC-derived hepatocytes), the one or more growth factors (e.g., an EGF, an FGF, an HGF, a TGF, or a combination thereof) is added to the cell culture medium when required, for example, daily or every other day. The one or more growth factors may be added singularly or in combination. In some embodiments, the one or more growth factors are added upon replacement of the cell culture medium (e.g., the expansion medium). In some embodiments, the one or more growth factors are added directly to the cell culture medium prior to adding the cell culture medium to the cells.

In some embodiments, the expansion medium includes a signal pathway agonist such as a Wnt signaling pathway agonist. The Wnt signaling pathway is an evolutionarily conserved developmental signaling pathway that is initiated upon activation of the cell-surface Wnt receptor complex, which includes a Frizzled receptor, a low-density lipoprotein receptor-related protein (LRP), and a leucine-rich repeat-containing G-protein-coupled receptor (LGR). The cell-surface Wnt receptor complex is usually activated by an extracellular signaling molecule, such as a member of the Wnt family, which then results in the activation of Disheveled family proteins, which inhibit a complex of proteins that includes axin, GSK-3, and APC to degrade intracellular β-catenin. The resulting enriched nuclear β-catenin enhances transcription by TCF/LEF family transcription factors. A Wnt agonist is an agent that activates TCF/LEF-mediated transcription in a cell. Wnt agonists are therefore selected from Wnt agonists that bind and activate the Wnt receptor complex including any and all of the Wnt family proteins, such as an inhibitor of intracellular β-catenin degradation, including a GSK inhibitor (such as the small molecule GSK3 inhibitor CHIR99021 (CAS No. 252917-06-9) and activators of TCF/LEF.

The Wnt agonist in the expansion medium may be any agonist able to stimulate the Wnt pathway via the LGR5 cell surface receptor, e.g., an LGR5 agonist. Known LGR5 agonists include an R-spondin protein and anti-LGR5 antibodies, including protein and antibody fragments that retain binding to LGR5. Exemplary LGR5 agonists are described in WO 2012/140274 and De Lau, W. et al. Nature. 476(7360):293-7, 2011, each of which is hereby incorporated by reference. In preferred embodiments, an LGR5 agonist added to the expansion medium is R-spondin. In some embodiments, the expansion medium includes an R-spondin, wherein the R-spondin includes R-spondin 1, R-spondin 2, R-spondin 3, R-spondin 4, or a combination thereof. In some embodiments, the R-spondin added to the expansion medium is a human R-spondin. In some embodiments, the R-spondin is a recombinant R-spondin (e.g., a recombinant human R-spondin; e.g., a recombinant human R-spondin 1). In some embodiments, one or more R-spondin proteins are added to the expansion medium at a concentration of 5 ng/ml to 1 μg/mL (e.g., 5 to 25 ng/ml, 5 to 50 ng/ml, 5 to 100 ng/ml, 5 to 150 ng/mL, 10 to 100 ng/ml, 10 to 150 ng/ml, 50 to 100 ng/ml, 50 to 150 ng/ml, 50 to 200 ng/ml, 150 to 300 ng/ml, 200 to 500 ng/ml, 500 to 600 ng/ml, 600 to 700 ng/ml, 700 to 800 ng/ml, 800 to 900 ng/ml, or 900 to 1 μg/mL; e.g., 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1,000 ng/ml). In some embodiments, the R-spondin added to the expansion medium comprises R-spondin 1. In some embodiments, R-spondin 1 and one or more additional R-spondin proteins (e.g., R-spondin 1 and R-spondin 2, R-spondin 3, R-spondin 4, or a combination thereof) are added to the expansion medium. In some embodiments, two R-spondin proteins are added to the expansion medium. In some embodiments, three R-spondin proteins are added to the expansion medium. In some embodiments, all four R-spondin proteins are added to the expansion medium. In some embodiments, the expansion medium does not include an R-spondin.

In some embodiments, a Wnt agonist is a secreted glycoprotein selected from the following: Wnt-I/Int-1, Wnt-2/Irp (InM-related protein), Wnt-2b/13, Wnt-3/Int-4, Wnt-3a, Wnt-4, Wnt-5a, Wnt-5b, Wnt-6 (Kirikoshi H et al., 2001 Biochem Biophys Res Com 283 798-805), Wnt-7a (R&D systems), Wnt-7b, Wnt-8a/8d, Wnt-8b, Wnt-9a/14, Wnt-9b/14b/15, Wnt-10a, Wnt-10b/12, WnMI, and Wnt-16. In preferred embodiments, Wnt3a is not added to the expansion medium. An overview of human Wnt proteins is provided in “THE WNT FAMILY OF SECRETED PROTEINS,” R&D Systems Catalog, 2004.

The Wnt agonist is preferably added to the expansion medium in an amount effective to stimulate Wnt activity in a cell. Wnt activity can be determined by measuring the transcriptional activity of Wnt, for example by TOPFLASH and FOPFLASH Tcf luciferase reporter constructs, such as those described in Korinek et al., Science. 275:1784-1787, 1997, which is hereby incorporated by reference. In some embodiments, a Wnt agonist is added to the expansion medium at a concentration of 10 ng/ml to 1 μg/mL (e.g., 10 to 100 ng/ml, 10 to 150 ng/ml, 50 to 100 ng/ml, 50 to 150 ng/ml, 50 to 200 ng/ml, 150 to 300 ng/ml, 200 to 500 ng/ml, 500 to 600 ng/ml, 600 to 700 ng/ml, 700 to 800 ng/ml, 800 to 900 ng/ml, or 900 to 1 μg/mL; e.g., 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1,000 ng/mL).

In some embodiments, during culturing of hepatocytes (e.g., PHHs or iPSC-derived hepatocytes), the one or more Wnt agonist such as one or more R-spondin proteins may be added to the culture medium when required, for example, daily or every other day. In some embodiments, the one or more Wnt agonists are added upon replacement of the cell culture medium (e.g., the expansion medium). In some embodiments, the Wnt agonist may be added to the culture medium prior to adding the cell culture medium to the cells.

iii. Transforming Growth Factor-Beta Inhibitor

The expansion medium described herein may include one or more transforming growth factor-beta (TGF-beta) inhibitors. A TGF-beta inhibitor may be any agent (e.g., a small molecule, a protein, or an inhibitory nucleic acid) that can attenuate or prevent the transcription of one or more genes that are transcribed due to the activity of a SMAD transcription co-activator protein. A TGF-beta inhibitor may disrupt the signal transduction cascade that leads to SMAD-induced gene transcription at one or more points within the pathway. For instance, a TGF-beta inhibitor may disrupt or prevent TGF-beta or a TGF-beta superfamily ligand, such as activin, inhibin, nodal, lefty, bone morphogenetic protein (BMP), growth and differentiation factor (GDF), or mullerian inhibitory factor (MIF), from binding to its endogenous receptor, thus inhibiting the phosphorylation and activation of the receptor-associated SMAD proteins.

The presence of a TGF-beta inhibitor in the expansion media is advantageous because it reduces the likelihood that the hepatocytes (e.g., PHHs or iPSC-derived hepatocytes) differentiate by reducing or inhibiting the activity of the TGF-beta signaling pathway, thereby preventing development of the mesenchymal phenotype. TGF-beta signaling is involved in many cellular functions, including cell growth, cell fate, and apoptosis. Signaling is generally initiated by the binding of a TGF-beta superfamily ligand to a type II receptor, which subsequently recruits and phosphorylates a type I receptor. The type I receptor then phosphorylates SMAD proteins, which act as transcription factors in the nucleus and regulate target gene expression.

The TGF-beta superfamily ligands include bone morphogenic proteins (BMPs), growth and differentiation factors (GDFs), anti-müllerian hormone (AMH), activin, nodal, and TGF-beta isoforms TGF-beta-1, TGF-beta-2, and TGF-beta-3. In general, SMAD2 and SMAD3 are phosphorylated by activin receptor-like kinases (ALK) ALK4, ALK5, and ALK7 in the TGF-beta/activin pathway. In contrast, SMAD1, SMAD5 and SMAD8 are phosphorylated as part of the bone morphogenetic protein (BMP) pathway. Although there is some cross-over between pathways, in the context of this disclosure, a “TGF-beta inhibitor” or an “inhibitor of TGF-beta signaling” is preferably an inhibitor of the TGF-beta pathway which acts via SMAD2 and SMAD3. Therefore, in some embodiments the TGF-beta inhibitor is not a BMP inhibitor such that, for example, the TGF-beta inhibitor is not Noggin.

Thus, the TGF-beta inhibitor may be any agent that reduces the activity of the TGF-beta signaling pathway. Many targets and inhibition strategies thereof of the TGF-beta signaling pathway are known in the art. For example, TGF-beta signaling may be dampened by any one or more of the following: inhibition of TGF-beta expression by a small-interfering RNA strategy; inhibition of furin (a TGF-beta activating protease); inhibition of the pathway by physiological inhibitors; neutralization of one or more TGF-beta isoforms with a monoclonal antibody; inhibition with small-molecule inhibitors of TGF-beta receptor kinase 1 (also known as ALK5), or other TGF-beta-related receptor kinases; inhibition of SMAD2 and SMAD3 signaling, e.g., by overexpression of their physiological inhibitor SMAD7 or by using thioredoxin as a SMAD anchor to inhibit SMAD activation (Fuchs, O. Inhibition of TGF-Signaling for the Treatment of Tumor Metastasis and Fibrotic Diseases. Current Signal Transduction Therapy, Volume 6, Number 1, January 2011, pp. 29-43(15)).

Various methods for determining whether a substance is a TGF-beta inhibitor are known in the art. For example, a cellular assay may be used in which cells are stably transfected with a reporter construct including the human PAI-1 promoter or SMAD binding sites, driving a luciferase reporter gene. Inhibition of luciferase activity relative to control groups can be used as a measure of compound activity (De Gouville et al., Br J Pharmacol. 145(2):166-177, 2005).

A TGF-beta inhibitor that is added to the cell culture medium (e.g., expansion medium) may be a protein, a peptide, a small molecule, a small interfering RNA, an antisense oligonucleotide, an aptamer, or an antibody. The inhibitor may be naturally occurring or synthetic. In some embodiments, the TGF-beta inhibitor is an inhibitor of ALK5. For example, the TGF-beta inhibitor may bind to and directly inhibit ALK5. In some embodiments, the TGF-beta inhibitor is A83-01 (CAS No. 909910-43-6), a small molecule that inhibits Smad signaling by inhibiting ALK4, ALK5, and ALK7.

In some embodiments, 500 nM to 5 μM A83-01 (e.g., 500 nM to 1 μM, 1 to 2 μM, 2 to 3 μM, 3 to 4 μM, or 4 to 5 μM; e.g., about 500 nM, about 600 nM, about 700 nM, about 800 nM, about 900 nM, about 1 μM, about 1.1 μM, about 1.2 μM, about 1.3 μM, about 1.4 μM, about 1.5 μM, about 1.6 μM, about 1.7 μM, about 1.8 μM, about 1.9 μM, about 2 μM, about 2.1 μM, about 2.2 μM, about 2.3 μM, about 2.4 μM, about 2.5 μM, about 2.6 μM, about 2.7 μM, about 2.8 μM, about 2.9 μM, about 3 μM, about 3.5 μM, about 4 μM, about 4.5 μM, or about 5 M) is added to the expansion medium.

In some embodiments, one TGF-beta inhibitor is added to the expansion medium. In some embodiments, more than one (e.g., two, three, four, or more) TGF-beta inhibitors are added to the expansion medium. In some embodiments, A83-01 is the only TGF-beta inhibitor added to the expansion medium. In some embodiments, A83-01 and one or more additional TGF-beta inhibitors are added to the expansion medium. In some embodiments, Noggin is not added to the expansion medium.

iv. Serum Replacement Component or Serum

An expansion medium described herein may include a serum replacement component or a serum.

In some embodiments, the expansion medium includes a serum or a serum replacement component (i.e., a component that may substitute fetal bovine serum as an additive in a cell culture medium). In some embodiments, the expansion medium includes a serum or a serum replacement component at a volumetric concentration of 0.1% to 20% (e.g., 0.1% to 1%, 0.1% to 5%, 0.1% to 10%, 1% to 5%, 1% to 10%, 1% to 20%, 5% to 10%, 5% to 20%, or 10% to 20%; e.g., about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9%, about 10%, about 11%, about 12%, about 13%, about 14%, about 15%, about 16%, about 17%, about 18%, about 19%, or about 20%).

In some embodiments, the volumetric concentration of the added serum or serum replacement component is unchanged over the course of culturing the hepatocytes (e.g., PHHs or iPSC-derived hepatocytes). In some embodiments, the volumetric concentration of the added serum or serum replacement component is changed over the course of culturing the hepatocytes (e.g., PHHs or iPSC-derived hepatocytes), such that the volumetric concentration of the added serum or serum replacement component is increased or decreased when replacing the cell culture medium (e.g., expansion medium). In some embodiments, the volumetric concentration of the added serum or serum replacement component is increased during culturing such that the volumetric concentration of the serum or serum replacement component increases as the cell density of the hepatocytes (e.g., PHHs or iPSC-derived hepatocytes) increases. Any suitable serum replacement component may be used. In some embodiments, the serum replacement component is KNOCKOUT™ Serum Replacement (KOSR), human platelet lysate, human serum, insulin transferrin selenium (ITS), Trace Elements A (i.e., a cell culture medium additive that includes copper, zinc, iron, and selenium), or Trace Elements B (i.e., a cell culture medium additive that includes ammonium molybdate, ammonium vanadate, manganese sulfate, nickel sulfate, sodium silicate, stannous chloride, and hydrochloric acid). KOSR is a serum-free eukaryotic cell culture medium supplement that includes or is obtained by combining albumin or an albumin supplement and one or more ingredients selected from the group consisting of glycine, L-histidine, L-isoleucine, L-methionine, L-phenylalanine, L-proline, L-hydroxyproline, L-serine, L-threonine, L-tryptophan, L-tyrosine, L-valine, thiamine, reduced glutathione, L-ascorbic acid-2-phosphate, iron saturated transferrin, insulin, and compounds containing the trace element moieties Ag⁺, Al³⁺, Ba²⁺, Cd²⁺, Co²⁺, Cr³⁺, Ge⁴⁺, Se⁴⁺, Br⁻, I⁻, Mn²⁺, F⁻, Si⁴⁺, V⁵⁺, Mo⁶⁺, Ni²⁺, Rb⁺, Sn²⁺ and Zr⁴⁺ (see, e.g., U.S. Publication No. US20020076747, the disclosure of which is hereby incorporated by reference in its entirety).

In some embodiments, the serum replacement component includes KOSR. In some embodiments, the expansion medium includes KOSR at a volumetric concentration of 0.1% to 20% (e.g., 0.1% to 1%, 0.1% to 5%, 0.1% to 10%, 1% to 5%, 1% to 10%, 1% to 20%, 5% to 10%, 5% to 20%, or 10% to 20%; e.g., about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9%, about 10%, about 11%, about 12%, about 13%, about 14%, about 15%, about 16%, about 17%, about 18%, about 19%, or about 20%).

In some embodiments, during culturing of hepatocytes (e.g., PHHs or iPSC-derived hepatocytes), the serum replacement component or serum may be added to the culture medium when required, for example, daily or every other day. In some embodiments, the serum replacement component or serum is added upon replacement of the cell culture medium (e.g., the expansion medium). In some embodiments, the serum replacement component or serum may be added to the culture medium prior to adding the cell culture medium to the cells.

In some embodiments, the concentration of the serum or serum replacement component is changed depending on the cell density or confluence of the hepatocytes during expansion.

In some embodiments, the volumetric concentration of the serum or serum replacement component is increased after seeding the hepatocytes. In some embodiments, the volumetric concentration of the serum or serum replacement component is increased from 1% to 6% (e.g., 1%, 2%, 3%, 4%, 5%, or 6%) to a volumetric concentration of 7% to 15% (e.g., 7%, 8%, 9%, 10%, 11%, 12%, 13%, 14%, or 15%) after seeding the hepatocytes. In some embodiments, the volumetric concentration of the serum or serum replacement component is increased when the cell density of the hepatocytes (e.g., PHHs or iPSC-derived hepatocytes) reaches 20% to 70% confluency (e.g., 20% to 30%, 30% to 40%, 40% to 50%, 50% to 60%, 60% to 70%; e.g., about 20%, about 25%, about 30%, about 35%, about 40%, about 45%, about 50%, about 55%, about 60%, about 65%, or about 70% confluency). In some embodiments, the volumetric concentration of the serum or serum replacement component is increased after 72 hours or after 120 hours (e.g., 120 to 170 hours) of culturing the hepatocytes. In some embodiments, the volumetric concentration of the serum or serum replacement component is increased at about 80 hours, about 90 hours, about 100 hours, about 120 hours, about 130 hours, about 140 hours, about 150 hours, about 160 hours, about 170 hours, about 180 hours, about 190 hours, or about 200 hours of culturing the hepatocytes (e.g., PHHs or iPSC-derived hepatocytes).

In some embodiments, the volumetric concentration of the serum or serum replacement component is increased at day 3, at day 4, at day 5, at day 6, at day 7, at day 8, at day 9, at day 10, at day 11, at day 12, at day 13, or at day 14 of culturing the hepatocytes (e.g., PHHs or iPSC-derived hepatocytes).

In some embodiments, the volumetric concentration of the added serum or serum replacement component is increased in the cell culture medium (e.g., expansion medium) more than once over the course of culturing the hepatocytes (e.g., PHHs or iPSC-derived hepatocytes). In some embodiments, the volumetric concentration of the added serum or serum replacement component is increased in the cell culture medium (e.g., expansion medium) in a single passage. In some embodiments, the volumetric concentration of the added serum or serum replacement component is increased in the cell culture medium (e.g., expansion medium) in every passage over the course of the culture (e.g., every passage for 2 passages, 3 passages, 4 passages, 5 passages, 6 passages, or more over the course of the culture).

In some embodiments the volumetric concentration of the added serum or serum replacement component is increased once, twice, three times, four times, or more than four times over the course of culturing the hepatocytes (e.g., PHHs or iPSC-derived hepatocytes). As an example, the volumetric concentration of the added serum or serum replacement component is 1% upon seeding the hepatocytes and is increased to 5% and then further increased to 10% over the course of culturing the hepatocytes in a cell culture medium.

In some embodiments, the serum or serum replacement component is added to the cell culture medium (e.g., the expansion medium) at a volumetric concentration of 1% to 6% (e.g., 1%, 2%, 3%, 4%, 5%, or 6%) upon seeding the hepatocytes in step P0. In some embodiments, the serum or serum replacement component is added to the cell culture medium (e.g., the expansion medium) at a volumetric concentration of 1% upon seeding the hepatocytes in step P0. In some embodiments, the serum or serum replacement component is added to the cell culture medium (e.g, the expansion medium) at a volumetric concentration of 5% upon seeding the hepatocytes in step P0.

In some embodiments, the volumetric concentration of the serum or serum replacement component is raised to 5% in the cell culture medium (e.g., the expansion medium) when the cell density reaches 15% to 30% confluency (e.g., about 15%, about 20%, about 25%, or about 30%) at step P0. In some embodiments, the volumetric concentration of the serum replacement component is raised to 5% in the cell culture medium (e.g., the expansion medium) at day 3 to day 7 (e.g., day 3, day 4, day 5, day 6, or day 7) of step P0.

In some embodiments, the volumetric concentration of the serum replacement component is raised to 10% when the cell density reaches 40% to 60% confluency (e.g., about 40%, about 45%, about 50%, about 55%, or about 60%) of step P0. In some embodiments, the volumetric concentration of the serum replacement component is raised to 10% at day 7 to day 13 (e.g., day 7, day 8, day 9, day 10, day 11, day 12, or day 13) of step P0.

In some embodiments, the serum replacement component is present in the cell culture medium (e.g., the expansion medium) at a volumetric concentration of 1% upon the passage of the hepatocytes in step P1. In some embodiments, the serum replacement component is present in the cell culture medium (e.g., the expansion medium) at a volumetric concentration of 5% upon the passage of hepatocytes in step P1.

In some embodiments, the volumetric concentration of the serum replacement component is raised to 5% in the cell culture medium (e.g., the expansion medium) when the cell density reaches 15% to 30% confluency (e.g., about 15%, about 20%, about 25%, or about 30%) at step P1. In some embodiments, the volumetric concentration of the serum replacement component is raised to 5% in the cell culture medium (e.g., the expansion medium) at day 3 to day 7 (e.g., day 3, day 4, day 5, day 6, or day 7) of step P1.

In some embodiments, the volumetric concentration of the serum replacement component is raised to 10% when the cell density reaches 40% to 60% confluency (e.g., about 40%, about 45%, about 50%, about 55%, or about 60%) of step P1. In some embodiments, the volumetric concentration of the serum replacement component is raised to 10% between day 5 to day 13 (e.g., day 5, day 6, day 7, day 8, day 9, day 10, day 11, day 12, or day 13) of step P1.

In some embodiments, the expansion medium includes a serum. In some embodiments, the serum includes fetal bovine serum. In some embodiments, the expansion medium includes a serum at a volumetric concentration of 0.1% to 20% (e.g., 0.1% to 1%, 0.1% to 5%, 0.1% to 10%, 1% to 5%, 1% to 10%, 1% to 20%, 5% to 10%, or 10% to 20%; e.g., about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9%, about 10%, about 11%, about 12%, about 13%, about 14%, about 15%, about 16%, about 17%, about 18%, about 19%, or about 20%). In preferred embodiments, the expansion medium does not include a serum.

v. Additional Medium Components

An expansion medium described herein optionally includes one or more additional agents or supplements that are added to the basal cell culture medium, such as an antioxidant, a cell survival agent, an amino acid supplement, a buffering agent, an antibiotic, or any combination thereof.

In some embodiments, the expansion medium includes an antioxidant. In some embodiments, the antioxidant is added to the expansion medium at a concentration of 100 μM to 10 mM (e.g., 100 M to 500 μM, 500 μM to 1 mM, 1 mM to 2 mM, 2 mM to 3 mM, 3 mM to 4 mM, 4 mM to 5 mM, 5 mM to 6 mM, 6 mM to 7 mM, 7 mM to 8 mM, 8 mM to 9 mM, or 9 mM to 10 mM; e.g., 100 μM, 200 μM, 300 μM, 400 μM, 500 μM, 600 μM, 700 μM, 800 μM, 900 μM, 1 mM, 1.25 mM, 1.5 mM, 1.75 mM, 2 mM, 2.25 mM, 2.5 mM, 2.75 mM, 3 mM, 3.25 mM, 3.5 mM, 3.75 mM, 4 mM, 4.25 mM, 4.5 mM, 4.75 mM, 5 mM, 5.25 mM, 5.5 mM, 5.75 mM, 6 mM, 6.25 mM, 6.5 mM, 6.75 mM, 7 mM, 7.25 mM, 7.5 mM, 7.75 mM, 7 mM, 7.25 mM, 7.5 mM, 7.75 mM, 8 mM, 8.25 mM, 8.5 mM, 8.75 mM, 9 mM, 9.25 mM, 9.5 mM, 9.75 mM, or 10 mM).

In some embodiments, the antioxidant includes N-acetyl cysteine. In some embodiments, the antioxidant includes nicotinamide. In some embodiments, the antioxidant includes both N-acetyl cysteine and nicotinamide.

In some embodiments, the expansion medium includes 100 μM to 5 mM N-acetylcysteine (e.g., 100 μM to 500 μM, 500 M to 1 mM, 1 mM to 2 mM, 2 mM to 3 mM, 3 mM to 4 mM, or 4 mM to 5 mM; e.g., 100 μM, 200 μM, 300 μM, 400 μM, 500 μM, 600 μM, 700 μM, 800 μM, 900 μM, 1 mM, 1.25 mM, 1.5 mM, 1.75 mM, 2 mM, 2.25 mM, 2.5 mM, 2.75 mM, 3 mM, 3.25 mM, 3.5 mM, 3.75 mM, 4 mM, 4.25 mM, 4.5 mM, 4.75 mM, or 5 mM).

In some embodiments, the expansion medium includes nicotinamide at a concentration of 10 ng/ml to 1 μg/mL (e.g., 10 ng/ml to 50 ng/ml, 50 ng/ml to 100 ng/mL, 100 ng/ml to 200 ng/ml, 200 ng/ml to 300 ng/ml, 300 ng/mL to 400 ng/ml, 400 ng/ml to 500 ng/ml, 500 ng/ml to 600 ng/ml, 600 ng/ml to 700 ng/ml, 700 ng/ml to 800 ng/ml, 800 ng/ml to 900 ng/ml, or 900 ng/ml to 1 μg/mL; 10 ng/ml, 20 ng/ml, 30 ng/ml, 40 ng/ml, 50 ng/ml, 60 ng/mL, 70 ng/ml, 80 ng/ml, 90 ng/mL, 100 ng/ml, 110 ng/ml, 120 ng/mL, 130 ng/ml, 140 ng/ml, 150 ng/ml, 200 ng/mL, 250 ng/ml, 300 ng/ml, 350 ng/ml, 400 ng/ml, 450 ng/ml, 500 ng/ml, 550 ng/ml, 600 ng/ml, 650 ng/ml, 700 ng/ml, 750 ng/ml, 800 ng/ml, 850 ng/mL, 900 ng/ml, 950 ng/ml, or 1 μg/mL).

In some embodiments, the expansion medium includes agents or additives that promote cell survival of the cell culture. In some embodiments, a cell survival agent is a supplement such as a supplement for a serum-free cell culture medium (i.e., a cell culture medium that does not contain animal serum, e.g., fetal bovine serum). In some embodiments, the supplement includes a B27 supplement. In some embodiments, the supplement includes an N2 supplement. In some embodiments, the supplement includes both a B27 supplement and an N2 supplement.

In some embodiments, the B27 does not contain vitamin A. The B27 supplement may be used to formulate a cell culture medium that includes biotin, cholesterol, linoleic acid, linolenic acid, progesterone, putrescine, retinyl acetate, sodium selenite, triiodothyronine (T3), DL-alpha tocopherol (vitamin E), albumin, insulin, and transferrin. The B27 supplement without vitamin A was shown to work particularly well in an expansion medium for liver cells (e.g., hepatocytes; e.g., PHHs or iPSC-derived hepatocytes). The B27 supplement may be purchased as a liquid 50× concentrate that comprises biotin, cholesterol, linoleic acid, linolenic acid, progesterone, putrescine, retinol, retinyl acetate, sodium selenite, tri-iodothyronine (T3), DL-alpha tocopherol (vitamin E), albumin, insulin, and transferrin. Of these ingredients, at least linolenic acid, retinol, retinyl acetate, and triiodothyronine (T3) are nuclear hormone receptor agonists. B27 supplement may be added to a culture medium as a concentrate or diluted before addition to a culture medium. It may be used at a 1× final concentration or at other concentrations. Use of B27 supplement is a convenient way to incorporate biotin, cholesterol, linoleic acid, linolenic acid, progesterone, putrescine, retinol, retinyl acetate, sodium selenite, tri-iodothyronine (T3), DL-alpha tocopherol (vitamin E), albumin, insulin, and transferrin into a culture medium of the invention. It is also envisaged that some or all of these components may be added separately to the expansion medium instead of using the B27 supplement. Thus, the expansion medium may include some or all of these components.

In some embodiments, the expansion medium includes a B27 supplement at a concentration of 0.1× to 100× (e.g., 0.1× to 90×, 0.5× to 80×, 1× to 70×, 5× to 60×, and 10× to 50×; e.g., about 0.1×, about 0.2×, about 0.3×, about 0.4×, about 0.5×, about 0.6×, about 0.7×, about 0.8×, about 0.9×, about 1×, about 1.5×, about 2×, about 2.5×, about 3×, about 3.5×, about 4×, about 4.5×, about 5×, about 6×, about 7×, about 8×, about 9×, about 10×, about 15×, about 20×, about 25×, about 30×, about 35×, about 40×, about 45×, about 50×, about 60×, about 70×, about 80×, about 90×, or about 100×). In some embodiments, the expansion medium includes an N2 supplement. N2 supplement may be purchased as a 100× liquid concentrate, containing human transferrin, bovine insulin, progesterone, putrescine, and sodium selenite. N2 supplement may be added to a culture medium as a concentrate or diluted before addition to a culture medium. It may be used at a 1× final concentration or at other concentrations. Use of N2 supplement is a convenient way to incorporate transferrin, insulin, progesterone, putrescine, and sodium selenite into a culture medium of the invention. It is also envisaged that some or all of these components may be added separately to the expansion medium instead of using the N2 supplement. Thus, in some embodiments, the expansion medium may include some or all of these components.

In some embodiments, the expansion medium includes an N2 supplement at a concentration of 0.1× to 100× (e.g., 0.1× to 90×, 0.5× to 80×, 1× to 70×, 5× to 60×, and 10× to 50×; e.g., about 0.1×, about 0.2×, about 0.3×, about 0.4×, about 0.5×, about 0.6×, about 0.7×, about 0.8×, about 0.9×, about 1×, about 1.5×, about 2×, about 2.5×, about 3×, about 3.5×, about 4×, about 4.5×, about 5×, about 6×, about 7×, about 8×, about 9×, about 10×, about 15×, about 20×, about 25×, about 30×, about 35×, about 40×, about 45×, about 50×, about 60×, about 70×, about 80×, about 90×, or about 100×).

In some embodiments, the expansion medium includes an amino acid supplement. In some embodiments, the amino acid supplement is a non-essential amino acid (NEAA) supplement that includes one or more amino acids. In some embodiments, the NEAA supplement includes glycine, L-alanine, L-asparagine, L-aspartic acid, L-glutamic acid, L-proline, L-serine, or any combination thereof. In some embodiments, the NEAA supplement includes glycine, L-alanine, L-asparagine, L-aspartic acid, L-glutamic acid, L-proline, and L-serine. In some embodiments, the expansion medium includes a non-essential amino acid supplement containing 1 M to 100 mM, e.g., 1 M to 10 μM (e.g., 1 μM, 2 μM, 3 μM, 4 μM, 5 μM, 6 μM, 7 μM, 8 μM, 9 μM, or 10 μM), 10 μM to 100 μM (e.g., 10 μM, 20 μM, 30 μM, 40 μM, 50 μM, 60 μM, 70 μM, 80 μM, 90 μM, or 100 M), 100 μM to 1 mm (e.g., 100 μM, 200 μM, 300 μM, 400 μM, 500 μM, 600 μM, 700 μM, 800 μM, 900 μM, or 1 mm), 1 mM to 10 mM (e.g., 1 mM, 2 mM, 3 mM, 4 mM, 5 mM, 6 mM, 7 mM, 8 mM, 9 mM, or 10 mM), or 10 mM to 100 mM (e.g., 10 mM, 20 mM, 30 mM, 40 mM, 50 mM, 60 mM, 70 mM, 80 mM, 90 mM, or 100 mM) of each of glycine, L-alanine, L-asparagine, L-aspartic acid, L-glutamic acid, L-proline, and L-serine. In some embodiments, the expansion medium includes glycine, L-alanine, L-asparagine, L-aspartic acid, L-glutamic acid, L-proline, L-serine, or any combination thereof. In some embodiments, one or more of and glycine, L-alanine, L-asparagine, L-aspartic acid, L-glutamic acid, L-proline, L-serine is added to the expansion medium at a concentration of 10 μM to 50 μM, 50 μM to 100 μM, 100 μM to 200 μM, 200 μM to 300 μM, 300 μM to 400 μM, 400 μM to 500 μM, 500 μM to 600 μM, 600 μM to 700 μM, 700 μM to 800 μM, 800 μM to 900 μM, or 900 μM to 1 mM. In some embodiments, the expansion medium includes 100 μM to 500 μM (e.g., 100, 125, 150, 175, 200, 225, 250, 275, 300, 325, 350, 375, 400, 425, 450, 475, or 500 μM) of each of glycine, L-alanine, L-asparagine, L-aspartic acid, L-glutamic acid, L-proline, and L-serine. In some embodiments, the NEAA supplement is purchased as a solution comprising glycine, L-alanine, L-asparagine, L-aspartic acid, L-glutamic acid, L-proline, and L-serine at a 100× concentration. In some embodiments, the expansion medium includes an NEAA supplement solution at a final concentration of 0.1× to 10× (e.g., 0.1× to 1×, 0.1× to 5×, 1× to 5×, 1× to 10×, or 5× to 10×; e.g., about 0.1×, about 0.2×, about 0.3×, about 0.4×, about 0.5×, about 0.6×, about 0.7×, about 0.8×, about 0.9×, about 1×, about 1.5×, about 2×, about 2.5×, about 3×, about 3.5×, about 4×, about 4.5×, about 5×, about 6×, about 7×, about 8×, about 9×, or about 10×).

In some embodiments, the expansion medium includes L-glutamine or an L-glutamine substitute or derivative thereof. In some embodiments, the L-glutamine is an L-glutamine supplement, e.g., L-alanyl-L-glutamine dipeptide, e.g., 200 mM L-alanyl-L-glutamine dipeptide in 0.85% NaCl (e.g., GLUTAMAX™). In some embodiments, the expansion medium includes L-glutamine or an L-glutamine substitute or derivative thereof (e.g., GLUTAMAX™) at a volumetric concentration of 0.1% to 20% (e.g., 0.1% to 1%, 0.1% to 5%, 0.1% to 10%, 1% to 5%, 1% to 10%, 1% to 20%, 5% to 10%, 5% to 20%, or 10% to 20%; e.g., about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9%, about 10%, about 11%, about 12%, about 13%, about 14%, about 15%, about 16%, about 17%, about 18%, about 19%, or about 20%). In some embodiments, L-glutamine or an L-glutamine substitute or derivative thereof is provided as a solution at a 100×concentration and is added at a concentration of 0.1× to 10× (e.g., 0.1× to 1×, 0.1× to 5×, 1× to 5×, 1× to 10×, or 5× to 10×; e.g., about 0.1×, about 0.2×, about 0.3×, about 0.4×, about 0.5×, about 0.6×, about 0.7×, about 0.8×, about 0.9×, about 1×, about 1.5×, about 2×, about 2.5×, about 3×, about 3.5×, about 4×, about 4.5×, about 5×, about 6×, about 7×, about 8×, about 9×, or about 10×).

In some embodiments, an expansion medium includes a buffering agent. A buffering agent may be any buffering agent suitable for mammalian cell culture. In some embodiments, the expansion medium includes a buffering agent at a concentration of 1 mM to 100 mM (e.g., 1 to 5 mM, 1 to 10 mM, 1 to 20 mM, 1 to 50 mM, 10 to 50 mM, 10 to 100 mM, 20 to 100 mM, or 50 to 100 mM; e.g., 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM).

In some embodiments, the buffering agent is HEPES. In some embodiments, the expansion medium includes 1 mM to 50 mM HEPES (e.g., 1 to 5 mM, 1 to 10 mM, 1 to 20 mM, 1 to 50 mM, 10 to 20 mM, 10 to 50 mM, or 20 to 50 mM; e.g., 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, or 50 mM).

In some embodiments, the expansion medium includes an antibiotic. In some embodiments, the expansion medium does not include an antibiotic. In some embodiments, the antibiotic includes penicillin, streptomycin, or both penicillin and streptomycin. In some embodiments, the expansion medium includes about 0.1% or more (e.g., about 0.5%, about 1%, or about 5%) of a solution of penicillin and streptomycin. In some embodiments, the expansion medium includes from 0.1% to 10%, e.g., 0.1% to 1% (e.g., 0.1%, 0.2%, 0.3%, 0.4%, 0.5%, 0.6%, 0.7%, 0.8%, 0.9%, or 1%) or 1% to 10% (e.g., 1%, 2%, 3%, 4%, 5%, 6%, 7%, 8%, 9%, or 10%) a solution of penicillin and streptomycin.

In some embodiments, during culturing of hepatocytes (e.g., PHHs or iPSC-derived hepatocytes), any one or more of the foregoing supplements and agents (e.g., an antioxidant, a cell survival agent, an amino acid supplement a buffering agent, and/or an antibiotic) may be added to the culture medium when required, for example, daily or every other day. In some embodiments, the one or more supplements and agents are added upon replacement of the cell culture medium (e.g., the expansion medium). In some embodiments, the one or more supplements and agents may be added to the culture medium prior to adding the cell culture medium to the cells.

In some embodiments, certain reagents may not be added to the cell culture medium for improved expansion or cost effectiveness. In some embodiments, one or more paracrine signaling hormones arachidonic acid, a prostaglandin (e.g., prostaglandin E2), gastrin, or a combination thereof are not added to the cell culture medium (e.g., the expansion medium). In some embodiments, arachidonic acid is not added to the cell culture medium. In some embodiments, prostaglandin E2 is not added to the cell culture medium. In some embodiments, gastrin is not added to the cell culture medium. In some embodiments, arachidonic acid and prostaglandin E2 are not added to the cell culture medium. In some embodiments, arachidonic acid and gastrin are not added to the cell culture medium. In some embodiments, arachidonic acid and prostaglandin E2 are not added to the cell culture medium. In some embodiments, arachidonic acid, prostaglandin, and gastrin are not added to the cell culture medium.

In some embodiments, one or more of FGF7, Wnt3a, and TGF-alpha are not added to the cell culture medium. In some embodiments, two or more of FGF7, Wnt3a, and TGF-alpha are not added to the basal cell culture medium. In some embodiments, FGF7, Wnt3a, and TGF-alpha are not added to the basal cell culture medium.

In some embodiments, two or more of gastrin, arachidonic acid, and prostaglandin E2 are not added to the cell culture medium. In some embodiments, gastrin, arachidonic acid, and prostaglandin E2 are not added to the cell culture medium.

In some embodiments, FGF7 and gastrin are not added to the cell culture medium. In some embodiments, FGF7 and arachidonic acid are not added to the cell culture medium. In some embodiments, FGF7 and prostaglandin E2 are not added to the cell culture medium. In some embodiments, Wnt3a and gastrin are not added to the cell culture medium. In some embodiments, Wnt3a and arachidonic acid are not added to the cell culture medium. In some embodiments, Wnt3a and prostaglandin E2 are not added to the cell culture medium. In some embodiments, TGF-alpha and gastrin are not added to the cell culture medium. In some embodiments, TGF-alpha and arachidonic acid are not added to the cell culture medium. In some embodiments, TGF-alpha and prostaglandin E2 are not added to the cell culture medium.

In some embodiments, FGF7, gastrin, and arachidonic acid are not added to the cell culture medium. In some embodiments, FGF7, gastrin, and prostaglandin E2 are not added to the cell culture medium. In some embodiments, FGF7, arachidonic acid, and prostaglandin E2 are not added to the cell culture medium. In some embodiments, Wnt3a, gastrin, and arachidonic acid are not added to the cell culture medium. In some embodiments, Wnt3a, gastrin, and prostaglandin E2 are not added to the cell culture medium. In some embodiments, Wnt3a, arachidonic acid, and prostaglandin E2 are not added to the cell culture medium. In some embodiments, TGF-alpha, gastrin, and arachidonic acid are not added to the cell culture medium. In some embodiments, TGF-alpha, gastrin, and prostaglandin E2 are not added to the cell culture medium. In some embodiments, TGF-alpha, arachidonic acid, and prostaglandin E2 are not added to the cell culture medium.

In some embodiments, FGF7, Wnt3a, and gastrin are not added to the cell culture medium. In some embodiments, FGF7, Wnt3a, and arachidonic acid are not added to the cell culture medium. In some embodiments, FGF7, Wnt3a, and prostaglandin E2 are not added to the cell culture medium. In some embodiments, FGF7, Wnt3a, gastrin, and arachidonic acid are not added to the cell culture medium. In some embodiments, FGF7, Wnt3a, gastrin, and prostaglandin E2 are not added to the cell culture medium. In some embodiments, FGF7, Wnt3a, gastrin, arachidonic acid, and prostaglandin E2 are not added to the cell culture medium.

In some embodiments, TGF-alpha, Wnt3a, and gastrin are not added to the cell culture medium. In some embodiments, TGF-alpha, Wnt3a, and arachidonic acid are not added to the cell culture medium. In some embodiments, TGF-alpha, Wnt3a, and prostaglandin E2 are not added to the cell culture medium. In some embodiments, TGF-alpha, Wnt3a, gastrin, and arachidonic acid are not added to the cell culture medium. In some embodiments, TGF-alpha, Wnt3a, gastrin, and prostaglandin E2 are not added to the cell culture medium. In some embodiments, TGF-alpha, Wnt3a, gastrin, arachidonic acid, and prostaglandin E2 are not added to the cell culture medium.

In some embodiments, FGF7, TGF-alpha, and gastrin are not added to the cell culture medium. In some embodiments, FGF7, TGF-alpha, and arachidonic acid are not added to the cell culture medium. In some embodiments, FGF7, TGF-alpha, and prostaglandin E2 are not added to the cell culture medium.

In some embodiments, FGF7, TGF-alpha, gastrin, and arachidonic acid are not added to the cell culture medium. In some embodiments, FGF7, TGF-alpha, gastrin, and prostaglandin E2 are not added to the cell culture medium. In some embodiments, FGF7, TGF-alpha, gastrin, arachidonic acid, and prostaglandin E2 are not added to the cell culture medium.

In some embodiments, FGF7, Wnt3a, TGF-alpha, gastrin, arachidonic acid, and prostaglandin E2 are not added to the cell culture medium.

### C. Culture Surface

In some examples of the methods described herein, the hepatocytes (e.g., PHHs or iPSC-derived hepatocytes) are cultured on a two-dimensional surface (e.g., a two-dimensional surface of a cell culture vessel). The cell culture surface can be made of any material suitable for culturing mammalian cells. For example, the surface may be a biocompatible and easily sterilized material such as plastic or other artificial polymer material. In some embodiments, the cell culture surface may contain plastic or glass. In some embodiments, the cells are grown in one plane.

Any number of materials can be used to form the cell culture surface including polyamides, polyesters, polystyrene, polypropylene, polyacrylates, polyvinyl compounds (e.g., polyvinylchloride), polycarbonate, polytetrafluoroethylene (PTFE), nitrocellulose, cotton, polyglyolic acid (PGA), cellulose, dextran, gelatin, glass, fluoropolymers, fluorinated ethylene propylene, polyvinylidene, polydimethylsiloxane, and silicon substrates (e.g., fused silica, polysilicon, or single silicon crystals), and the like. Metals such as gold, silver, or titanium films may also be used.

In some embodiments, the surface may be modified to promote cellular adhesion (e.g., coated with a material that promotes engagement with a cell surface, e.g., via engagement of an extracellular cell adhesion protein or receptor). For example, a glass surface may be treated with a protein or a peptide to promote cell adhesion to the cell culture surface. In some embodiments, a single protein is adhered to the surface. In some embodiments, two or more proteins are adhered to the surface. In some embodiments, the surface is coated with an extracellular matrix (ECM) (e.g., an ECM described herein) to facilitate cell adhesion.

In some embodiments, the cell culture surface may be modified to promote cell proliferation (e.g., coated with a material that promotes proliferation via activation of one or more cell surface receptors). For example, a glass surface may be treated with a protein or a peptide to promote proliferation of one or more cells (e.g., a hepatocyte) via downstream signaling following activation of one or more cell surface receptors upon plating the one or more cells on the treated surface.

In some embodiments, the surface is coated with an ECM to promote cell adhesion and/or cell proliferation. In some embodiments, the ECM promotes cell adhesion and/or cell proliferation through an integrin-mediated pathway (e.g., via activity of integrins such as α1β1, α2β1. α3β1, α6β1, α6β4, and/or α7β1 integrins). In some embodiments, the ECM promotes cell adhesion and/or cell proliferation through a non-integrin-mediated pathway (e.g., via activity of non-integrin cellular surface proteins; e.g., DAG1, RPSA, and/or BCAM). In some embodiments, the ECM promotes cell adhesion and/or cell proliferation through both an integrin-mediated pathway and a non-integrin-mediated pathway.

In some embodiments, the hepatocytes (e.g., PHHs or iPSC-derived hepatocytes) are cultured on a two-dimensional surface, wherein the two-dimensional surface has a surface area of 9.5 cm² to 10,000 cm² (e.g., 9.5 to 500 cm², 500 to 1,000 cm², or 1,000 cm² to 10,000 cm²; e.g., 9.5 cm², 100 cm², 200 cm², 300 cm², 400 cm², 500 cm², 600 cm², 700 cm², 800 cm², 900 cm², 1,000 cm², 2,000 cm², 3,000 cm², 4,000 cm², 5,000 cm², 6,000 cm², 7,000 cm², 8,000 cm², 9,000 cm², or 10,000 cm²). In some embodiments, the PHHs are cultured on a two-dimensional surface, wherein the two-dimensional surface has a surface area of 600 cm² to 6,000 cm² (e.g., 600 cm², 650 cm², 700 cm², 750 cm², about 800 cm², 850 cm², 900 cm², 950 cm², 1,000 cm², 1,500 cm², 2,000 cm², 2,500 cm², 3,000 cm², 3,500 cm², 4,000 cm², 4,500 cm², 5,000 cm², 5,500 cm², or 6,000 cm²).

### D. Extracellular Matrix

As described herein, the methods for culturing hepatocytes (e.g., PHHs or iPSC-derived hepatocytes) may include culturing the one or more hepatocytes in contact with an extracellular matrix (ECM). In some embodiments, the hepatocytes contact the ECM through physical means, chemical means, or any combination thereof. Any suitable ECM may be used. Isolated hepatocytes (e.g., PHHs or iPSC-derived hepatocytes) are preferably cultured in a microenvironment that mimics, at least in part, a cellular niche in which the hepatocytes naturally reside. A cellular niche is determined in part by the hepatocytes (e.g., PHHs or iPSC-derived hepatocytes) and surrounding cells, and the ECM that is produced by the cells in said niche. This cellular niche may be mimicked by culturing the one or more hepatocytes (e.g., PHHs or iPSC-derived hepatocytes) in the presence of biomaterials, such as an ECM that provides key regulatory signals that control or maintain hepatocyte fate.

In some embodiments, the cell culture surface is coated with an ECM. In some embodiments, the hepatocytes (e.g., PHHs or iPSC-derived hepatocytes) adhere to the ECM. In some embodiments, the hepatocytes adhere to the ECM and are cultured on the surface of the ECM.

ECM includes a variety of polysaccharides, proteoglycans, water, elastin, and glycoproteins, wherein the glycoproteins may include a laminin, a collagen, an entactin (nidogen), a vitronectin, and/or a fibronectin. ECM components are secreted by connective tissue cells. Different types of ECM are known, including different compositions including different types of glycoproteins and/or different combinations of glycoproteins.

An ECM may be a one glycoprotein or a particular isoform thereof, or a purified or partially purified fraction that is enriched for ECM species based on their molecular weight (e.g., high molecular weight or low molecular weight species). An ECM may include a mixture of components, such as a mixture of glycoproteins. An ECM may include a laminin (e.g., laminin-111, laminin-211, laminin-121, laminin-221, laminin-332, laminin-311, laminin-321, laminin-411, laminin-421, laminin-511, laminin-521, and/or laminin-213), a collagen (e.g., type I, type II, type III, type IV, type V, type VI, type VII, type VIII, type IX, type X, type XI, type XII, type XIII, type XIV, type XV, type XVI, type XVII, type XVIII, type XIX, type XX, type XXI, type XXII, type XIII, and/or type XXIV collagen), a vitronectin (e.g., type I, type II, and/or type III vitronectin), a fibronectin (e.g., type I, type II, and/or type III fibronectin), or any combination thereof.

An ECM can be provided by culturing ECM-producing cells, such as for example fibroblasts, in a receptacle, prior to the removal of these cells and the addition of isolated tissue fragments (e.g., tissue fragments that have been isolated from a liver) or isolated hepatocytes (e.g., PHHs or iPSC-derived hepatocytes). Examples of ECM-producing cells are chondrocytes that primarily produce collagen and proteoglycans; fibroblasts that primarily produce type IV collagen, laminin, interstitial procollagens, and fibronectin; and colonic myofibroblasts that primarily produce type I, type III, and type V collagens, chondroitin sulfate proteoglycan, hyaluronic acid, fibronectin, and tenascin-C.

Alternatively, an ECM can be commercially provided. Examples of commercially available ECMs are ECM proteins (Invitrogen). A synthetic ECM material may be used. Mixtures of ECM materials may be used, if desired. In some embodiments, the ECM does not include a hydrogel (e.g., MATRIGEL™). In some embodiments, the ECM includes a hydrogel (e.g., MATRIGEL™). In some embodiments, the hydrogel is MATRIGEL™.

In some embodiments, the ECM includes a xeno-free substrate. In some embodiments, the ECM includes one or more recombinant proteins (e.g., recombinant glycoproteins).

In some embodiments, the ECM includes collagen. In some embodiments, the collagen is type I collagen or type IV collagen. In some embodiments, the collagen is type I collagen. In some embodiments, the collagen is type IV collagen.

In some embodiments, the ECM includes laminin. In some embodiments, the laminin is laminin-111, laminin-211, laminin-221, laminin-332, laminin-411, laminin-421, laminin-511, or laminin-521. In some embodiments, the laminin is laminin-111. In some embodiments, the laminin is laminin-511. In some embodiments, the laminin is laminin-521.

In some embodiments, the ECM is applied to a surface (e.g., the surface of a cell culture vessel). In some embodiments, the ECM is applied to the surface at a density of 0.050 μg/cm² to 0.150 μg/cm² (e.g., 0.050, 0.055, 0.060, 0.065, 0.070, 0.075, 0.080, 0.085, 0.090, 0.095, 0.100, 0.105, 0.110, 0.115, 0.120, 0.125, 0.130, 0.135, 0.140, 0.145, or 0.150 μg/cm²). In some embodiments, the ECM is applied to the surface at a density of 0.150 μg/cm² to 1 μg/cm² (e.g., 0.150 μg/cm² to 0.300 μg/cm², 0.150 μg/cm² to 0.500 μg/cm², 0.200 g/cm² to 0.600 μg/cm², or 0.600 g/cm² to 1 g/cm²; e.g., 0.160, 0.170, 0.180, 0.190, 0.200, 0.210, 0.220, 0.230, 0.240, 0.250, 0.300, 0.350, 0.400, 0.450, 0.500, 0.550, 0.600, 0.650, 0.700, 0.750, 0.800, 0.850, 0.900, 0.950, or 1 g/cm²).

In some embodiments, the culture medium is placed on top of the ECM.

In some embodiments, a population of cells (e.g., hepatocytes) is in contact with an ECM or a scaffold that mimics the ECM (e.g., a polymer) by via cellular membrane proteins, such as, e.g., integrins (e.g., α1β1, α2β1. α3β1, α6β1, α6β4, and/or α7β1 integrins) or non-integrin cellular membrane proteins (e.g., dystroglycan (DAG1), ribosomal protein SA (RPSA) or laminin receptor, and/or basal cell adhesion molecule (BCAM)). In some embodiments, a population of cells contacts an ECM or a scaffold that mimics the ECM only through basal membrane interactions when cultured in a 2D culture system. In some embodiments, a population of cells contacts an ECM or a scaffold that mimics the ECM through basal and apical membrane interactions (i.e., contacts on multiple surfaces of a cell) when embedded in an ECM or a scaffold in a 3D culture system.

### E. Hypoxic Culture Conditions

The culture methods described herein may include culturing hepatocytes (e.g., PHHs or iPSC-derived hepatocytes) under hypoxic conditions or in the presence of a hypoxic mimetic. Hypoxic conditions, as described herein, include any condition where oxygen is present in concentrations below normal oxygen concentrations (normoxic conditions). Hypoxic mimetics mimic hypoxia by inducing the accumulation of hypoxia-inducible factor one alpha (HiF1α), which is a protein subunit of a transcription factor that responds to decreases in available oxygen.

In some embodiments, culturing includes culturing the cells under hypoxic conditions. Hypoxic conditions may include, e.g., an oxygen level of less than 20.9%. In some embodiments, the culturing under hypoxic conditions includes culturing the cells at an oxygen level of 1% to 20.8% (e.g., 2.5% to 19%, e.g., 2.5% to 10%, e.g., 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, or 20.5%). In some embodiments, the culturing under hypoxic conditions includes culturing the cells at an oxygen level of 2.5% to 19%. In some embodiments, the culturing includes culturing the cells at an oxygen level of 2.5% to 10%. In some embodiments, the culturing includes culturing the cells at an oxygen level of 5%. In some embodiments, the culturing includes culturing the cells under normoxic conditions.

In some embodiments, culturing includes culturing the cells in the presence of a hypoxic mimetic (e.g., a HIF-1α stabilizer or a PHD inhibitor). Exemplary hypoxia mimetics are an iron chelator (e.g., deferoxamine (DFO), compound A, deferasirox, and 2,2′-dipyridyl (DP)), an ion competitor (e.g., cobalt chloride (CoCl₂) or a divalent metal ion such as Ni²⁺, Mn²⁺, Co²⁺, or Zn²⁺), and a 2 oxoglutarate (2OG) analog (e.g., dihydroxybenzoic acid (DHB), N-oxalylglycine, dimethyloxalylglycine (DMOG)), a prolyl hydroxylase domain (PHD (inhibitor (e.g., FG-4497, GSK360A, TM6008, or folic acid), or a HIF-1a stabilizer (e.g., miR-335, isoflurane, N-acetylcysteine, MG-132, BSc2118, and tilorone).

Hypoxia mimetics are also described, e.g., in Davis et al. Front. Cell Dev. Biol. 6:175, 2018, which is hereby incorporated by reference. In some embodiments, the culturing in the presence of a hypoxic mimetic includes culturing the cells in the presence of cobalt chloride. In some embodiments, the culturing in the presence of a hypoxic mimetic includes culturing the cells in the presence of DFO. In some embodiments, the culturing in the presence of a hypoxic mimetic includes culturing the cells in the presence of DMOG. In some embodiments, the culturing includes culturing the cells in the absence of a hypoxic mimetic.

### F. Hepatocyte Maturation

Methods of the disclosure may include maturing a population of hepatocytes (e.g., PHHs or iPSC-derived hepatocytes), such as a population of expanded hepatocytes. In some embodiments, maturation of a population of hepatocytes (e.g., PHHs or iPSC-derived hepatocytes) occurs immediately after hepatocyte expansion (e.g., a method of hepatocyte expansion described herein). In some embodiments, maturation of a population of hepatocytes (e.g., PHHs or iPSC-derived hepatocytes) does not occur immediately after hepatocyte expansion. In some embodiments, a population of expanded hepatocytes have been cryopreserved prior to maturation.

In some embodiments, the maturation step has a duration of 3 to 14 days (e.g., 3 to 7 days, 3 to 10 days, 5 to 10 days, 7 to 10 days, 7 to 14 days, or 10 to 14 days; e.g., 3, 4, 5, 6, 7, 8, 9, 10, 11, 12, 13, or 14 days). In some embodiments, the maturation step is 7 days.

In some embodiments, the population of hepatocytes is matured on a surface of a culture vessel (e.g., in contact with a surface of a culture vessel or in contact with an ECM that is coated on a surface of a culture vessel). In some embodiments, the population of hepatocytes is matured in a suspension culture, in which the hepatocytes are cultivated via dispersion (e.g., by shaking or agitation) in a maturation medium. In some embodiments, the specific components that are included in a maturation medium depend on the culture format (i.e., maturation on a cell culture surface or in a suspension culture as a seed). For example, a population of cells that includes hepatocytes in a seed format may be encapsulated in a hydrogel during maturation to further enhance cell-cell interactions.

In some embodiments, the population of hepatocytes is matured in hypoxic conditions, in which the hepatocytes are cultured under normoxic conditions (e.g., atmospheric oxygen levels of greater than 20.9%, e.g., atmospheric oxygen levels of 21% or more).

In some embodiments, the population of hepatocytes is matured in vivo, such as following administration of hepatocytes (e.g., expanded hepatocytes) into a subject (e.g., a human subject).

### G. Maturation Medium

A maturation medium of the disclosure is composed of a basal cell culture medium to which one or more maturation supplements are added. The maturation medium may be used for cultivating hepatocytes (e.g., expanded hepatocytes; e.g., PHHs or iPSC-derived hepatocytes) in vitro. Components of a maturation medium are described below.

i. Basal Cell Medium

The basal cell culture medium of the maturation medium may be a commercially available basal cell culture medium that is suitable for the in vitro cultivation of mammalian (e.g., human) cells. In some embodiments, the basal cell culture medium is Takara Cellartis POWER™ Primary HEP medium. In some embodiments, the basal cell culture medium is Lonza HCM™ cell culture medium. In some embodiments, the basal cell culture medium is William's E medium. In some embodiments, the basal cell culture medium is LIFENET HEALTH® Human Hepatocyte Media.

ii. Maturation Supplements

In some embodiments, the one or more maturation supplements includes a buffering agent that is suitable for mammalian cell culture. In some embodiments, the maturation medium includes a buffering agent at a concentration of 1 mM to 100 mM (e.g., 1 to 5 mM, 1 to 10 mM, 1 to 20 mM, 1 to 50 mM, 10 to 50 mM, 10 to 100 mM, 20 to 100 mM, or 50 to 100 mM; e.g., 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM). In some embodiments, the buffering agent is HEPES.

In some embodiments, the maturation medium includes L-glutamine or an L-glutamine substitute or derivative thereof. In some embodiments, the L-glutamine is an L-glutamine supplement, e.g., L-alanyl-L-glutamine dipeptide, e.g., 200 mM L-alanyl-L-glutamine dipeptide in 0.85% NaCl (e.g., GLUTAMAX™). In some embodiments, the maturation medium includes GLUTAMAX™ at a volumetric concentration of 0.1% to 20% (e.g., 0.1% to 1%, 0.1% to 5%, 0.1% to 10%, 1% to 5%, 1% to 10%, 1% to 20%, 5% to 10%, 5% to 20%, or 10% to 20%; e.g., about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9%, about 10%, about 11%, about 12%, about 13%, about 14%, about 15%, about 16%, about 17%, about 18%, about 19%, or about 20%). In some embodiments, L-glutamine or an L-glutamine substitute or derivative thereof is provided as a solution at a 100×concentration and is added at a concentration of 0.1× to 10× (e.g., 0.1× to 1×, 0.1× to 5×, 1× to 5×, 1× to 10×, or 5× to 10×; e.g., about 0.1×, about 0.2×, about 0.3×, about 0.4×, about 0.5×, about 0.6×, about 0.7×, about 0.8×, about 0.9×, about 1×, about 1.5×, about 2×, about 2.5×, about 3×, about 3.5×, about 4×, about 4.5×, about 5×, about 6×, about 7×, about 8×, about 9×, or about 10×).

In some embodiments, the maturation medium includes a Notch inhibitor. A Notch inhibitor may be any agent that inhibits or decreases signaling activity of the Notch signaling pathway, e.g., by inhibiting or decreasing activity of a Notch receptor (e.g., NOTCH1, NOTCH2, NOTCH3, and/or NOTCH4). A Notch inhibitor may reduce cell surface expression of one or more Notch receptors or reduce the expression of Notch Intracellular Domain (NICD) induced by the transmembrane ligand Delta-like 4 (DLL4). A Notch inhibitor may be an antibody that targets one or more Notch receptors. A Notch inhibitor may be a small molecule inhibitor.

In some embodiments, a Notch inhibitor is a gamma secretase inhibitor, which inhibits proteolytic cleavage of a Notch receptor intracellular domain. Gamma secretase inhibitors are known in the art and are described elsewhere, such as Golde et al., Biochim Biphys Acta. 1828(12):2898-907, 2013, which is hereby incorporated by reference. In some embodiments, the Notch inhibitor includes gamma secretase inhibitor Compound E (CAS No. 209986-17-4). In some embodiments, the Notch inhibitor includes Gamma Secretase Inhibitor XX (CAS No. 209985-56-5). In some embodiments, the Notch inhibitor includes both Compound E and Gamma Secretase Inhibitor XX.

In some embodiments, the maturation medium includes an epidermal growth factor receptor (EGFR) inhibitor. In some embodiments, the maturation medium includes an EGFR inhibitor at a concentration of 200 nM to 20 μM (e.g., 200 nM to 1 μM, 500 nM to 5 μM, 1 μM to 5 μM, 1 μM to 10 μM, 5 μM to 10 μM, or 10 μM to 20 μM; e.g., 200 nM, 300 nM, 400 nM, 500 nM, 600 nM, 700 nM, 800 nM, 900 nM, 1 μM, 1.5 μM, 2 μM, 2.5 μM, 3 μM, 3.5 μM, 4 μM, 4.5 μM, 5 μM, 6 μM, 7 μM, 8 μM, 9 μM, 10 μM, 11 M, 12 μM, 13 μM, 14 μM, 15 μM, 16 μM, 17 μM, 18 μM, 19 μM, or 20 M). In some embodiments, the EGFR inhibitor is erlotinib hydrochloride.

In some embodiments, the maturation medium includes oncostatin M. In some embodiments, the maturation medium includes oncostatin M at a concentration of 1 to 200 ng/ml (e.g., 1 to 20 ng/ml, 10 to 30 ng/ml, 20 to 50 ng/ml, 50 to 75 ng/mL, 60 to 80 ng/ml, 80 to 100 ng/ml, 100 to 125 ng/mL, 125 to 150 ng/ml, 150 to 175 ng/ml, or 175 to 200 ng/mL; e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95, 100, 110, 120, 130, 140, 150, 160, 170, 180, 190, or 200 ng/mL).

In some embodiments, the maturation medium includes an antioxidant. In some embodiments, the maturation medium includes the antioxidant at a concentration of 0.1 mM to 10 mM (e.g., 0.1 mM to 1 mM, 1 mM to 5 mM, or 5 mM to 10 mM; e.g., 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM). In some embodiments, the antioxidant includes vitamin C.

In some embodiments, the maturation medium includes a glucocorticoid. In some embodiments, the glucocorticoid includes dexamethasone, hydrocortisone, prednisone, triamcinolone, methylprednisolone, or a combination thereof. In some embodiments, the maturation medium includes two or more glucocorticoids (e.g., two, three, four, or more). In some embodiments, the maturation medium includes a glucocorticoid at a concentration of 1 μM to 1 mM (e.g., 1 M to 5 μM, 1 μM to 10 μM, 1 μM to 50μ, 1 μM to 100μ, 100 μM to 200μ, 100 μM to 500 μM, 100 μM to 1 mM, or 500 μM to 1 mM; e.g., 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 30 μM, 40 μM, 50 μM, 60 μM, 70 μM, 80 μM, 90 μM, 100 M, 150 μM, 200 μM, 250 μM, 300 μM, 350 μM, 400 μM, 450 μM, 500 μM, 550 μM, 600 μM, 650 μM, 700 μM, 750 μM, 800 μM, 850 μM, 900 μM, or 1 mM). In some embodiments, the maturation medium includes dexamethasone. In some embodiments, the maturation medium includes hydrocortisone. In some embodiments, the maturation medium includes both dexamethasone and hydrocortisone.

In some embodiments, the maturation medium includes a pregnane X receptor (PXR) activator. PXR is also known in the art as the steroid and xenobiotic sensing nuclear receptor (SXR) and is a ligand-activated nuclear receptor. PXR may be activated by a hydrophobic ligand such as a hydrophobic vitamin, a bile acid (e.g., a bile acid described herein), or a combination thereof. In some embodiments, the PXR activator is a hydrophobic vitamin such as vitamin A, vitamin D, vitamin E, or vitamin K. In some embodiments, the PXR activator is vitamin K. The maturation medium may include a PXR activator at a concentration of 1 to 100 μM (e.g., 1 to 5 μM, 1 to 10 μM, 1 to 20 μM, 5 to 20 μM, 10 to 50 μM, or 50 to 100 μM; e.g., 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, or 100 μM).

In some embodiments, the maturation medium includes a bile acid. In some embodiments, the bile acid is a secondary bile acid. In some embodiments, the bile acid includes lithocholic acid, urso deoxycholic acid, or a combination thereof. In some embodiments, the maturation medium includes a bile acid at a concentration of 1 to 100 μM (e.g., 1 to 5 μM, 1 to 10 μM, 1 to 20 μM, 5 to 20 μM, 10 to 50 μM, or 50 to 100 μM; e.g., 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, or 100 M).

In some embodiments, the maturation medium includes a cholesterol.

In some embodiments, the maturation medium includes a cAMP or a CAMP analog. In some embodiments, the CAMP analog includes 8-bromo CAMP. In some embodiments, the CAMP analog includes forskolin. In some embodiments, the CAMP analog includes both 8-bromo cAMP and forskolin. In some embodiments, the maturation medium includes cAMP or a CAMP analog at a concentration of 1 to 100 μM (e.g., 1 to 5 μM, 1 to 10 μM, 1 to 20 μM, 5 to 20 μM, 10 to 50 μM, or 50 to 100 μM; e.g., 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, or 100 μM). In some embodiments, the maturation medium includes CAMP or a cAMP analog at a concentration of 0.1 to 10 mM (e.g., 0.1 to 1 mM, 0.1 to 5 mM, 1 to 5 mM, 1 to 10 mM, 1 to 20 mM, 1 to 50 mM, 10 to 50 mM, 10 to 100 mM, 20 to 100 mM, or 50 to 100 mM; e.g., 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM).

In some embodiments, the maturation medium includes a thyroid hormone. In some embodiments, the thyroid hormone is triiodothyronine (T3). In some embodiments, the maturation medium includes a thyroid hormone at a concentration of 1 to 30 μM (e.g., 1 to 5 μM, 1 to 10 μM, 10 to 20 μM, or 10 to 30 μM; e.g., 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, or 30 μM).

In some embodiments, the maturation medium includes a peptide hormone. In some embodiments, the peptide hormone includes glucagon or glucagon-like peptide-1 (GLP-1). In some embodiments, the peptide hormone includes glucagon. In some embodiments, the peptide hormone includes GLP-1. In some embodiments, the maturation medium includes the peptide hormone at a concentration of 10 nM to 1 μM (e.g., 10 nM to 50 nM, 10 nM to 100 nM, 50 nM to 200 nM, 50 nM to 500 nM, 100 nM to 500 nM, 100 nM to 1 μM, or 500 nM to 1 μM; e.g., 10 nM, 20 nM, 30 nM, 40 nM, 50 nM, 60 nM, 70 nM, 80 nM, 90 nM, 100 nM, 110 nM, 120 nM, 130 nM, 140 nM, 150 nM, 160 nM, 170 nM, 180 nM, 190 nM, 200 nM, 225 nM, 250 nM, 275 nM, 300 nM, 325 nM, 350 nM, 375 nM, 400 nM, 425 nM, 450 nM, 475 nM, 500 nM, 525 nM, 550 nM, 575 nM, 600 nM, 625 nM, 650 nM, 675 nM, 700 nM, 725 nM, 750 nM, 775 nM, 800 nM, 825 nM, 850 nM, 875 nM, 900 nM, 925 nM, 950 nM, 975 nM, or 1 μM).

In some embodiments, the maturation medium includes an amino acid supplement. In some embodiments, the amino acid supplement is an NEAA supplement or an essential amino acid (EAA) supplement. In some embodiments, the maturation medium includes both an NEAA supplement and an EAA supplement. In some embodiments, the NEAA supplement includes glycine, L-alanine, L-asparagine, L-aspartic acid, L-glutamic acid, L-proline, L-serine, or any combination thereof. In some embodiments, the NEAA supplement includes glycine, L-alanine, L-asparagine, L-aspartic acid, L-glutamic acid, L-proline, and L-serine. In some embodiments, the EAA supplement includes L-histidine, L-isoleucine, L-leucine, L-lysine, L-methionine, L-phenylalanine, L-threonine, L-tryptophan, L-valine, or any combination thereof. In some embodiments, the EAA supplement includes L-histidine, L-isoleucine, L-leucine, L-lysine, L-methionine, L-phenylalanine, L-threonine, L-tryptophan, and L-valine. In some embodiments, the amino acid supplement is provided as a solution at a 50× or 100× stock concentration. In some embodiments, the maturation medium includes the amino acid supplement at a final concentration of about 0.1× to about 100× (e.g., about 0.1× to about 90×, about 0.5× to about 80×, about 1× to about 70×, about 5× to about 60×, and about 10× to about 50×; e.g., about 0.1×, about 0.2×, about 0.3×, about 0.4×, about 0.5×, about 0.6×, about 0.7×, about 0.8×, about 0.9×, about 1×, about 1.5×, about 2×, about 2.5×, about 3×, about 3.5×, about 4×, about 4.5×, about 5×, about 6×, about 7×, about 8×, about 9×, about 10×, about 15×, about 20×, about 25×, about 30×, about 35×, about 40×, about 45×, about 50×, about 60×, about 70×, about 80×, about 90×, or about 100×).

In some embodiments, the maturation medium includes an antibiotic. In some embodiments, the maturation medium does not include an antibiotic. In some embodiments, the antibiotic includes penicillin, streptomycin, or both penicillin and streptomycin. In some embodiments, the maturation medium includes about 0.1% or more (e.g., about 0.5%, about 1%, or about 5%) of a solution of penicillin and streptomycin. In some embodiments, the maturation medium includes from 0.1% to 10%, e.g., 0.1% to 1% (e.g., 0.1%, 0.2%, 0.3%, 0.4%, 0.5%, 0.6%, 0.7%, 0.8%, 0.9%, or 1%) or 1% to 10% (e.g., 1%, 2%, 3%, 4%, 5%, 6%, 7%, 8%, 9%, or 10%) a solution of penicillin and streptomycin.

In some embodiments, the maturation medium includes additional additives. In some embodiments, the maturation medium includes additional additives when the population of hepatocytes is matured as a seed. In some embodiments, the maturation medium includes a ROCK inhibitor, such as Y-27632 (CAS No. 129830038-2). In some embodiments, the maturation medium includes a nuclease (e.g., a DNase or an RNase). In some embodiments, the maturation medium includes a DNase. In some embodiments, the maturation medium includes a surfactant such as a non-ionic detergent to reduce shear forces applied to cells in a suspension culture. In some embodiments, the non-ionic detergent includes Pluronic F68, Pluronic F127, Poloxamer 188, Poloxamer 407, polysorbate 20, polysorbate 80, or sorbitan monolaurate. In some embodiments, the non-ionic detergent includes Pluronic F68. For instance, a nuclease (e.g., a DNase) and a surfactant (e.g., a non-ionic detergent; e.g., Pluronic F68) may be added to a maturation medium to mature a population of hepatocytes that are cultured as a seed which the population of hepatocytes has been admixed with at least one additional cell type (e.g., a population of stromal cells such as fibroblasts (e.g., NHDF)).

In some embodiments, the maturation medium does not include one or more paracrine signaling hormones of arachidonic acid, a prostaglandin (e.g., prostaglandin E2), gastrin, or a combination thereof such that one or more of these paracrine signaling hormones are not added to the maturation medium. In some embodiments, arachidonic acid is not added to the cell culture medium. In some embodiments, the maturation medium does not include prostaglandin E2. In some embodiments, the maturation medium does not include gastrin. In some embodiments, the maturation medium does not include arachidonic acid and prostaglandin E2. In some embodiments, the maturation medium does not include arachidonic acid and gastrin. In some embodiments, the maturation medium does not include arachidonic acid and prostaglandin E2. In some embodiments, the maturation medium does not include arachidonic acid, prostaglandin, and gastrin.

iii. Serum Replacement Component

The maturation medium may include a serum replacement component, in which any suitable serum replacement component may be used. In some embodiments, the serum replacement component is KOSR, human platelet lysate, human serum, ITS, Trace Elements A (i.e., a cell culture medium additive that includes copper, zinc, iron, and selenium), or Trace Elements B (i.e., a cell culture medium additive that includes ammonium molybdate, ammonium vanadate, manganese sulfate, nickel sulfate, sodium silicate, stannous chloride, and hydrochloric acid). In some embodiments, the serum replacement component includes a combination of two or more (e.g., two, three, or four) of the foregoing serum replacement components. In some embodiments, the maturation medium includes KOSR and ITS.

### H. Cellular Signatures and Biomarkers

Following expansion and/or maturation of a population of hepatocytes (e.g., PHHs or iPSC-derived hepatocytes), the hepatocytes may be evaluated for expression of biomarkers that confirm hepatocyte cellular identity and function. Such biomarkers include proteins that are secreted into the bloodstream, such as coagulation factors, surface-expressed biomarkers and receptors, and liver enzymes, including urea cycle enzymes. Exemplary biomarkers that are expressed following expansion and/or maturation (e.g., via the expansion and maturation methods described herein) are discussed below.

Albumin is a globular protein that is produced by the liver and secreted into the bloodstream, in which it stabilizes osmotic pressure in the vasculature and carries endogenous ligands to various tissues. Albumin is a biomarker of both hepatoblasts (i.e., hepatic progenitor cells) and terminally differentiated hepatocytes. In some embodiments, after culturing, the population of hepatocytes secrete albumin. In general, expanded hepatocytes (e.g., PHHs or iPSC-derived hepatocytes) may secrete lower levels of albumin as compared to overnight plated control PHHs. In some embodiments, after maturation, the mature hepatocytes secrete albumin.

In some embodiments, the expanded and/or matured hepatocytes (e.g., PHHs or iPSC-derived hepatocytes) secrete 1 to 50 μg/million cells/day of albumin (e.g., 1 to 5 μg/million cells/day, 1 to 10 μg/million cells/day, 5 to 25 μg/million cells/day, 10 to 25 μg/million cells/day, 25 to 50 μg/million cells/day, or more than 50 μg/million cells/day; e.g., 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, or more than 50 μg/million cells/day). In some embodiments, at least 70% of hepatocytes in a population of expanded and/or matured hepatocytes secrete albumin. In some embodiments, at least 75%, at least 80%, at least 85%, or at least 90% of hepatocytes in a population of expanded and/or matured hepatocytes secrete albumin.

In some embodiments, the expanded and/or matured hepatocytes (e.g., PHHs or iPSC-derived hepatocytes) secrete coagulation factor IX. In some embodiments, the expanded and/or matured hepatocytes secrete 1 to 50 ng/million cells/day of factor IX (e.g., 1 to 5 ng/million cells/day, 1 to 10 ng/million cells/day, 5 to 25 ng/million cells/day, 10 to 25 ng/million cells/day, 25 to 50 ng/million cells/day, or more than 50 ng/million cells/day; e.g., 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, or more than 50 ng/million cells/day). In some embodiments, at least 70% of hepatocytes in a population of expanded and/or matured hepatocytes secrete factor IX. In some embodiments, at least 75%, at least 80%, at least 85%, or at least 90% of hepatocytes in a population of expanded and/or matured hepatocytes secrete factor IX.

In some embodiments, the expanded and/or matured hepatocytes express one or more other biomarkers that confirms hepatocyte identity. In some embodiments, the expanded and/or matured hepatocytes express hepatocyte biomarkers including HNFα, NR112, SERPINA1, alpha-1 antitrypsin, ASGR1, or a combination thereof.

In some embodiments, the expanded and/or matured hepatocytes express one or more biomarkers associated with liver metabolic function. In some embodiments, the expanded and/or matured hepatocytes secrete urea or express one or more biomarkers associated with the urea cycle, such as a gene encoding an enzyme in the urea cycle. The expanded and/or matured hepatocytes may express one or more urea cycle biomarkers including CYP protein family (e.g., CYP3A4, CYP1A2, or both), ARG1, ASL, ASS1, CPS1, NAGS, OTC, AQP9, SLC24A13, SLC25A15, or a combination thereof. In some embodiments, the expanded and/or matured hepatocytes express one or more glutamine synthesis biomarkers including GLS2, GLUL, GPX1, UGT1A1, or a combination thereof. In some embodiments, at least 70%, at least 75%, at least 80%, at least 85%, or at least 90% of hepatocytes in a population of expanded and/or matured hepatocytes express one or more urea cycle biomarkers.

In some embodiments, the expanded and/or matured hepatocytes express one or more biomarkers associated with cell polarity, such as apical and basolateral polarity membrane proteins. In some embodiments, the expanded and/or matured hepatocytes express ABCG2, ABCC2, ABCC3, ABCB11, SCARB1, ALC10A1, or a combination thereof.

In some embodiments, the expanded and/or matured hepatocytes do not express hepatic progenitor or cholangiocyte biomarkers. In some embodiments, the expanded and/or matured hepatocytes do not have detectable expression of hepatic progenitor or cholangiocyte biomarkers including AFP, CYP3A7, EPCAM, LGR5, KRT7, KRT19, AQP1, or a combination thereof. In some embodiments, a population of expanded hepatocytes (e.g., PHHs or iPSC-derived hepatocytes) have increased expression of hepatic progenitor or cholangiocyte markers such as AFP, CYP3A7, EPCAM, LGR5, KRT7, KRT19, AQP1, or a combination thereof as compared to a population of PHHs that were not expanded. In some embodiments, a population of expanded PHHs require a maturation step to downregulate or reduce the expression of hepatic progenitor or cholangiocyte biomarkers. In some embodiments, less than 10% (e.g., less than 10%, less than 9%, less than 8%, less than 7%, less than 6%, less than 5%, less than 4%, less than 3%, less than 2%, less than 1%) of hepatocytes in a population of expanded and/or matured hepatocytes express a hepatic progenitor or cholangiocyte biomarker.

The presence and/or expression level of cell biomarkers (e.g., biomarkers of hepatocyte identity or function described herein) may be detected or measured by any suitable methodology known in the art for evaluating the expression of nucleic acids such as RNA (e.g., mRNA transcripts), proteins (e.g., intracellularly expressed proteins, surface-expressed proteins, or secreted proteins), or metabolic products or byproducts (e.g., urea). In some embodiments, expression level of a protein (e.g., an enzyme) includes detecting or measuring its activity (e.g., by product yield and/or by a fluorescent or luminescent reporter assay).

Exemplary methods of detecting or measuring the relative expression of one or more transcription factors or biomarkers are flow cytometry, fluorescence activated cell sorting (FACS), massively parallel DNA sequencing (e.g., next-generation sequencing), RNA-sequencing (RNA-seq) (e.g., bulk RNA-seq or single-cell RNA-seq), real-time reverse transcription polymerase chain reaction (RT-PCR), quantitative PCR (qPCR), RT-qPCR, Northern blot analysis, mass spectrometry and proteomic modalities, Western blot analysis, enzyme-linked immunosorbent assay (ELISA), immunofluorescence or immunodetection methods, in situ hybridization (e.g., fluorescence in situ hybridization (FISH)) among other detection methods known in the art.

In some embodiments, the presence and/or expression level of a biomarker in a first sample (e.g., a cell such as an expanded hepatocyte or a matured hepatocyte, a tissue such as a portion of a liver, or a sample of culture medium such as culture medium that has been in contact with a population of hepatocytes) is compared to the expression level of the biomarker in a second sample. In some embodiments, the second sample is a reference sample such as, a reference cell or population of cells (e.g., PHHs or iPSC-derived hepatocytes cultured in the absence of a culture medium such as a culture medium described herein), a reference tissue, a control sample, a control cell, or a control tissue.

### I. Hepatocyte Aggregates

The expanded and/or matured hepatocytes (e.g., PHHs or iPSC-derived hepatocytes) may be aggregated. Aggregates described herein include a population of hepatocytes. In some embodiments, the hepatocytes are admixed under conditions which cause the cell population to form aggregates. In some embodiments, the hepatocytes are admixed using tissue fabrication techniques. In some embodiments, the hepatocytes are cultured by hanging drop, microwell molding, non-adhesive surfaces, spheroid suspension culture using a spinner flask, vertical wheel bioreactor, horizontal wheel bioreactor, or a microfluidic spheroid system. Additional methods include those using acoustical waves and using positively charged surfaces on a plate. In some embodiments, the hepatocytes (e.g., PHHs or iPSC-derived hepatocytes) are admixed in the presence of stromal cells (e.g., fibroblasts, e.g., NHDFs). In some embodiments, the hepatocytes (e.g., PHHs or iPSC-derived hepatocytes) are admixed in the presence of NHDFs. In some embodiments, the hepatocytes (e.g., PHHs or iPSC-derived hepatocytes) are admixed in the absence of stromal cells (e.g., NHDFs).

In some embodiments, an aggregate comprises 10 to 1,000 hepatocytes (e.g., 10 to 100, 50 to 500, 100 to 500, 100 to 1,000, or 500 to 1,000 hepatocytes; e.g., about 10, about 20, about 50, about 100, about 150, about 200, about 250, about 300, about 350, about 400, about 450, about 500, about 550, about 600, about 650, about 700, about 750, about 800, about 850, about 900, about 950, or about 1,000 hepatocytes).

In other aspects, the compositions provided herein can contain additional components, growth factors, ligands, cytokines, drugs, and the like. In some embodiments, the cell mixture can include molecules which elicit additional microenvironmental cues such as small molecules or growth factors which stimulate or enhance proliferation and expansion of a cell population.

In certain embodiments, the aggregates disclosed herein include one or more adherence materials to facilitate maintenance of the desired phenotype of the engrafted cells in vivo. The material may include an antibody, a protein or peptide, a nucleic acid, an aptamer (e.g., an RNA or DNA aptamer), a carbohydrate, a proteoglycan, or a matrix. The type of adherence materials (e.g., ECM materials, sugars, proteoglycans, etc.) will be determined, in part, by the cell type (e.g., PHHs or iPSC-derived hepatocytes) to be cultured.

In some embodiments, organizing cells and material into spatial arrangements, such as aggregates, can be accomplished by physically constraining the placement of cells/material by the use of wells or grooves, or injecting cells into microfluidic channels or oriented void spaces/pores. In certain embodiments, the cells can be organized by physically positioning cells with electric fields, magnetic tweezers, optical tweezers, ultrasound waves, pressure waves, or micromanipulators.

The cells produced by the culturing methods described herein can be used immediately in the making of an aggregate. Alternatively, the cells can be frozen in liquid nitrogen and stored for long periods of time for thawing and later making an aggregate. For example, the cells can be frozen in a culture medium that includes a cryoprotective agent, such as a medium that includes 10% dimethylsulfoxide (DMSO), 50% serum, 40% buffered medium, or one or more other agents known in the art.

### J. Cell Implants

As described herein, the methods disclosed herein may include introducing the population of expanded and/or matured hepatocytes (e.g., PHHs or iPSC-derived hepatocytes) or progeny thereof into a subject. In some embodiments, the population of expanded and/or matured hepatocytes or progeny thereof is introduced into a subject in the form of a hepatocyte aggregate. In some embodiments, the subject is a human. In some embodiments, the subject is a human patient suffering from a liver disease. The population of expanded and/or matured hepatocytes (e.g., PHHs or iPSC-derived hepatocytes) or progeny thereof may be incorporated into an engineered tissue construct, e.g., for implantation into a subject. The engineered tissue construct may include a biocompatible hydrogel scaffold (e.g., a scaffold that includes fibrin). The biocompatible scaffold may contain an encapsulated population of aggregated hepatocytes.

In some embodiments, uses of the population of expanded and/or matured hepatocytes (e.g., PHHs or iPSC-derived hepatocytes) cultured as described herein are likewise provided. For example, in some embodiments, the invention also provides the use of the population of expanded and/or matured hepatocytes of the invention or a lot of frozen hepatocytes derived from said population of expanded hepatocytes in a discovery screen; toxicity assay; gene expression studies including recombinant gene expression; research of mechanisms involved in tissue injury and repair; research of inflammatory and infectious diseases; studies of pathogenetic mechanisms; or studies of mechanisms of cell transformation and etiology of liver disease.

In some embodiments, the invention also provides cells derived from the population of expanded and/or matured hepatocytes (e.g., PHHs or iPSC-derived hepatocytes) of the invention for use in medicine. In some embodiments, the invention also provides cells derived from the population of expanded and/or matured hepatocytes of the invention for use in treating a disorder, condition, or disease. In some embodiments, the invention also provides the population of expanded hepatocytes, or cells derived from the population of expanded hepatocytes of the invention, for use in regenerative medicine, for example, wherein the use involves implantation of the population of expanded cells or cells derived from the population of expanded hepatocytes into a subject. In some embodiments, the invention also provides the population of matured hepatocytes (e.g., PHHs or iPSC-derived hepatocytes), or cells derived from the population of matured hepatocytes of the invention, for use in regenerative medicine, for example, wherein the use involves implantation of the population of matured cells or cells derived from the population of matured hepatocytes into a subject.

In some embodiments, a hepatocyte aggregate comprises 7×10⁶ to 8×10⁸ hepatocytes (e.g., expanded and/or matured hepatocytes, e.g., expanded and/or matured PHHs or iPSC-derived hepatocytes). In some embodiments, a hepatocyte aggregate comprises about 7×10⁶, about 7.5×10⁶, about 8×10⁶, about 8.5×10⁶, about 9×10⁶, about 1×10⁷, about 1.5×10⁷, about 2×10⁷, about 2.5×10⁷, about 3×10⁷, about 3.5×10⁷, about 4×10⁷, about 4.5×10⁷, about 5×10⁷, about 5.5×10⁷, about 6×10⁷, about 6.5×10⁷, about 7×10⁷, about 7.5×10⁷, about 8×10⁷, about 8.5×10⁷, about 9×10⁷, about 9.5×10⁷, about 1×10⁸, about 1.5×10⁸, about 2×10⁸, about 2.5×10⁸, about 3×10⁸, about 3.5×10⁸, about 4×10⁷, about 4.5×10⁸, about 5×10⁸, about 5.5×10⁸, about 6×10⁸, about 6.5×10⁸, about 7×10⁸, about 7.5×10⁸, or about 8×10⁸ hepatocytes.

### K. Pharmaceutical Compositions

The invention also provides a pharmaceutical formulation including one or more population of expanded and/or matured hepatocytes (e.g., PHHs or iPSC-derived hepatocytes) and a pharmaceutically acceptable diluent and/or excipient.

The cells or aggregates prepared by one or more of the methods described herein may be formulated into various compositions (e.g., a pharmaceutical composition) for administration to a subject in a biologically compatible form suitable for administration in vivo. For example, the cells (e.g., expanded and/or matured hepatocytes) or aggregates of the cells described herein may be administered in a suitable diluent, carrier, stabilizer, or excipient, and may further contain a preservative, e.g., to prevent the growth of microorganisms. Conventional procedures and ingredients for the selection and preparation of suitable compositions are described, for example, in Remington, J. P. The Science and Practice of Pharmacy, Easton, PA. Mack Publishers, 2012, 22nd ed. and in The United States Pharmacopeial Convention, The National Formulary, United States Pharmacopeial, 2015, USP 38 NF 33.

The cells or aggregates described herein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is hereby incorporated by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In some embodiments, a pharmaceutical composition is prepared for administration by injection, implantation, or engraftment. In some embodiments, a pharmaceutical composition includes a carrier and is formulated in aqueous solution, such as water or a physiologically compatible buffer such as Hanks's solution, Ringer's solution, or a saline buffer (e.g., phosphate buffered saline). In some embodiments, other ingredients are included (e.g., ingredients that aid in solubility or serve as preservatives). In some embodiments, injectable suspensions are prepared using appropriate liquid carriers, suspending agents and the like. Certain pharmaceutical formulations for injection may be presented in unit dosage form, e.g., in a vial.

### L. Kits

The compositions described herein can be provided in a kit for use in expanding freshly harvested or previously cryopreserved hepatocytes (e.g., PHHs or iPSC-derived hepatocytes) or for maturing hepatocytes, such as hepatocytes that have been expanded. In some embodiments, the kit can include one or more cell culture medium components or additives as described herein. In some embodiments, the kit further includes a package insert that instructs a user of the kit, such as a laboratory scientist, to perform any one of the methods described herein. In some embodiments, the kit includes cryopreserved hepatocytes (e.g., PHHs or iPSC-derived hepatocytes) that were previously expanded according to the method described herein. In some embodiments, the kit includes cryopreserved hepatocytes (e.g., PHHs or iPSC-derived hepatocytes) that were previously matured according to a method described herein. In some embodiments, the kit can include equipment for administering a pharmaceutical composition comprising hepatocytes (e.g., a pharmaceutical composition comprising expanded and/or matured hepatocytes).

### EXAMPLES

The following examples are set forth so as to provide those of ordinary skill in the art with a description of how the compositions and methods described herein may be used and evaluated and are intended to be purely exemplary of the invention and are not intended to limit the scope of what the inventors regard as their invention.

### Example 1. Evaluation of Cell Culture Method Using Variable Expansion Medium Formulations

This example describes expansion medium formulations developed by the inventors for expansion of hepatocytes.

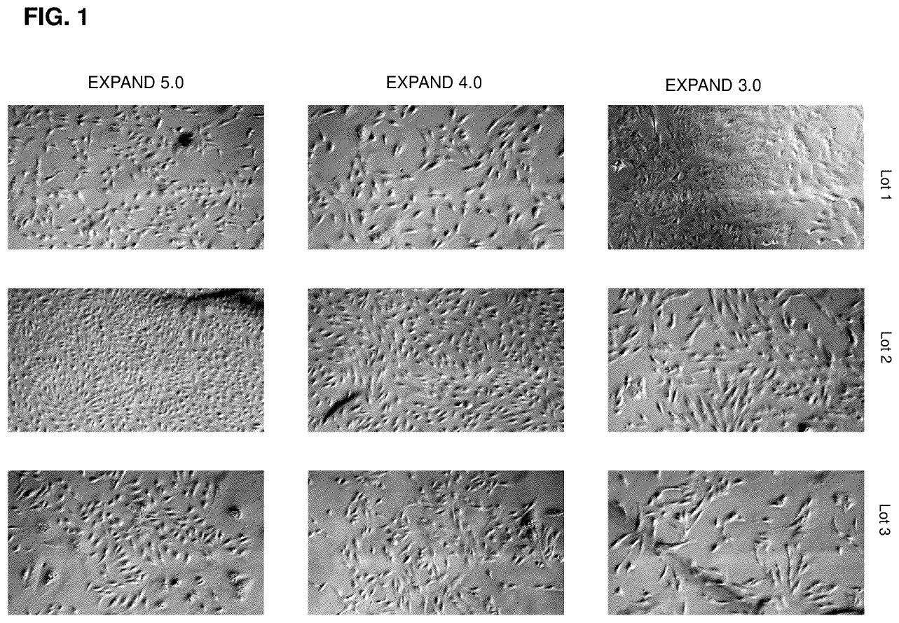

Three lots of primary human hepatocytes (PHHs) were expanded in expansion medium formulations Expand 3.0, Expand 4.0, or Expand 5.0, the components of which are shown in Table 1. PHHs were expanded for thirteen days in 6-well plates that were coated in laminin-521. PHHs cultured in the Expand 3.0 medium were plated at a density of 667 cells/cm², and PHHs cultured in the Expand 4.0 and Expand 5.0 media were plated at a density of 1,300 cells/cm². The expansion medium was refreshed every 48 hours over the course of the expansion. Representative brightfield images showing the morphology of the hepatocytes in each culture condition are shown in FIG. 1.

TABLE 1
Expansion Media Compositions

| | Components | EXPAND 3.0 Cocktail | | EXPAND 4.0 Cocktail | | EXPAND 5.0 Cocktail | |
|---|---|---|---|---|---|---|---|
| 1 | DMEM based media | Base Medium | | Base Medium | | Base Medium | |
| 2 | Penicillin/Streptomycin | 1% | | 1% | | 1% | |
| 3 | L-glutamine alternative | 1% | | 1% | | 1% | |
| 4 | HEPES | 10 | mM | 10 | mM | 10 | mM |
| 5 | B27 Supplement | 1X | | 1X | | 1X | |
| 6 | N2 Supplement | 1X | | 1X | | Not added | |
| 7 | Rspo-1 | 100 | ng/mL | 50 | ng/mL | Not added | |
| 8 | Wnt3a | 50 | ng/mL | Not added | | Not added | |
| 9 | N-acetylcysteine | 1.25 | mM | 1.25 | mM | 1.25 | mM |
| 10 | Nicotinamide | 100 | ng/mL | 100 | ng/mL | 100 | ng/mL |
| 11 | Recombinant EGF | 50 | ng/mL | 50 | ng/mL | 50 | ng/mL |
| 12 | Recombinant TGF-alpha | 20 | ng/mL | Not added | | 20 | ng/mL |
| 13 | Human FGF-7 | 50 | ng/mL | Not added | | Not added | |
| 14 | Human FGF-10 | 50 | ng/mL | 50 | ng/mL | 50 | ng/mL |
| 15 | Human HGF | 25 | ng/mL | 25 | ng/mL | 25 | ng/mL |
| 16 | A83-01 | 2 | μM | 2 | μM | 2 | μM |

| | | | | |
|---|---|---|---|---|
| 17 | KOSR | 1-5-10% or 5-10% | 1-5-10% or 5-10% | 1-5-10% or 5-10% |
| 18 | NEAA (100X) | 1X | 1X | 1X |
| | Hypoxic culture | Yes | Yes | Yes |
| | conditions? | | | |

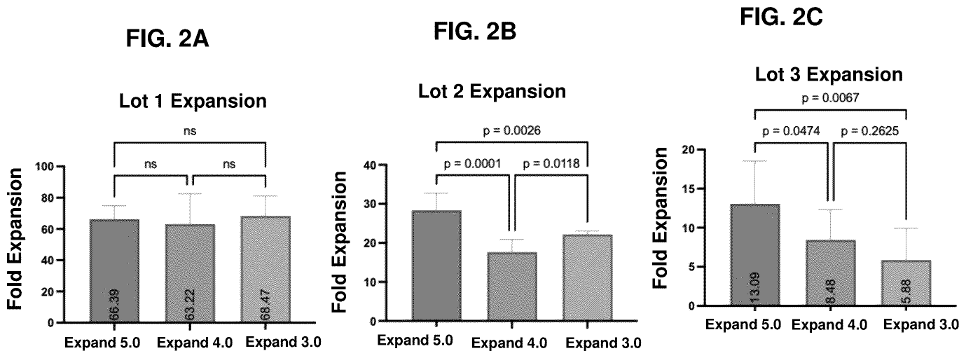

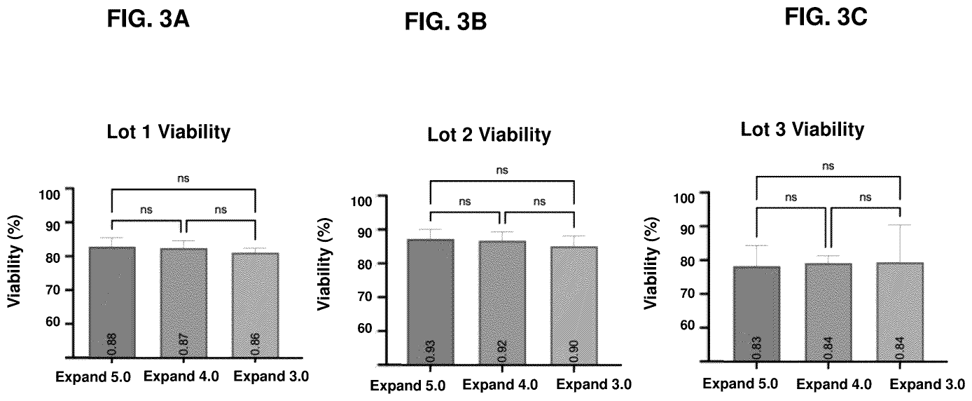

After confirming that cell morphology and hepatocyte expansion were comparable in the 6-well plate format, the expansion was repeated at a larger scale in T75 cell culture flasks. Hepatocyte expansion (FIGS. 2A-2C) and hepatocyte viability (FIGS. 3A-3C) of each cell lot were evaluated after the thirteen-day expansion.

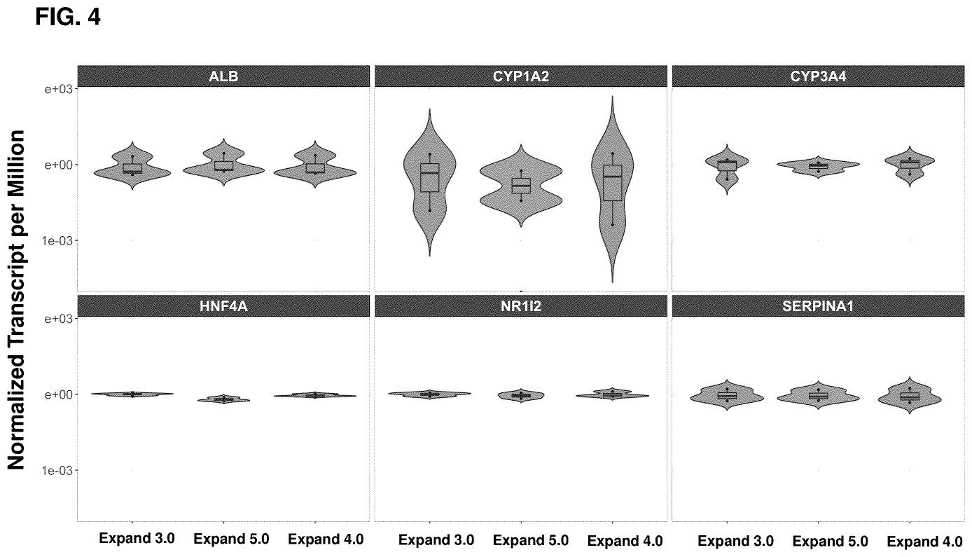

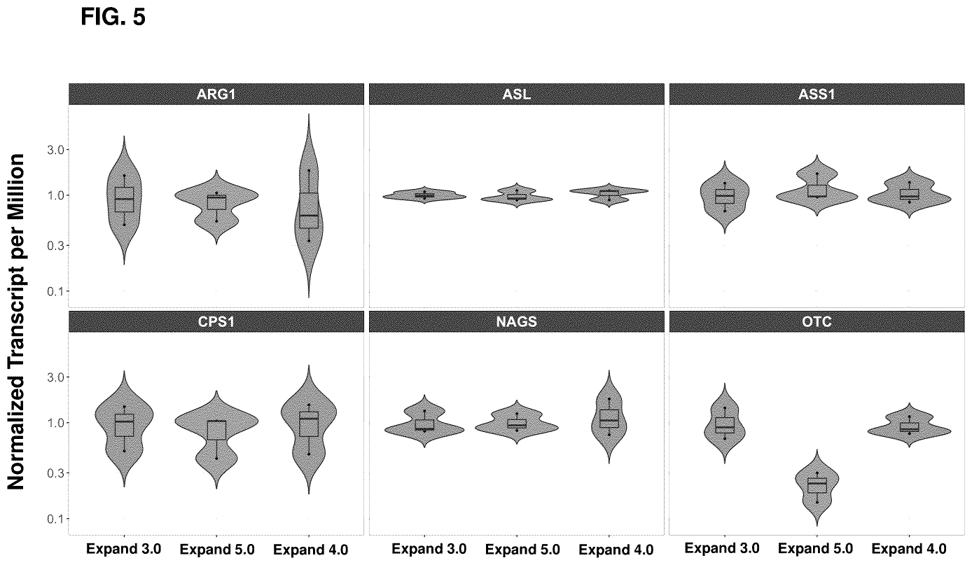

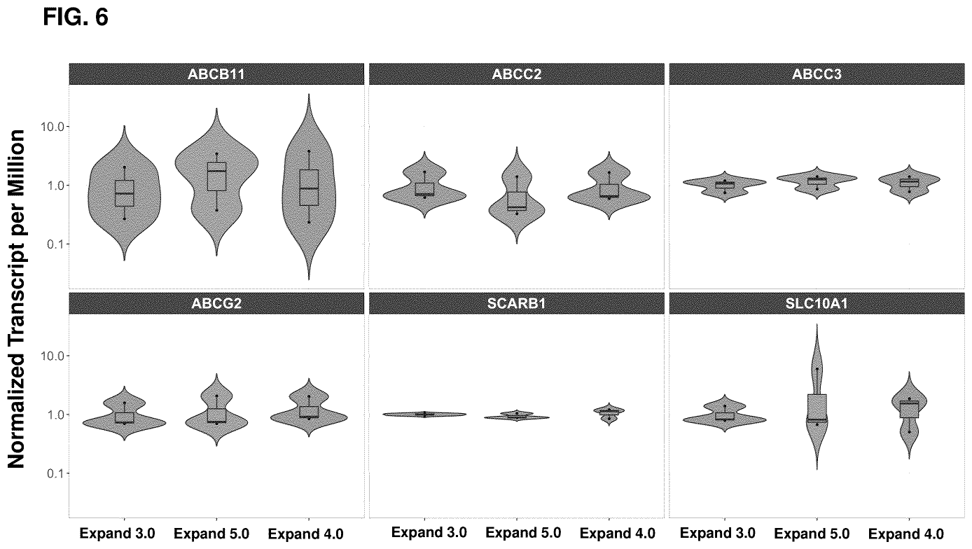

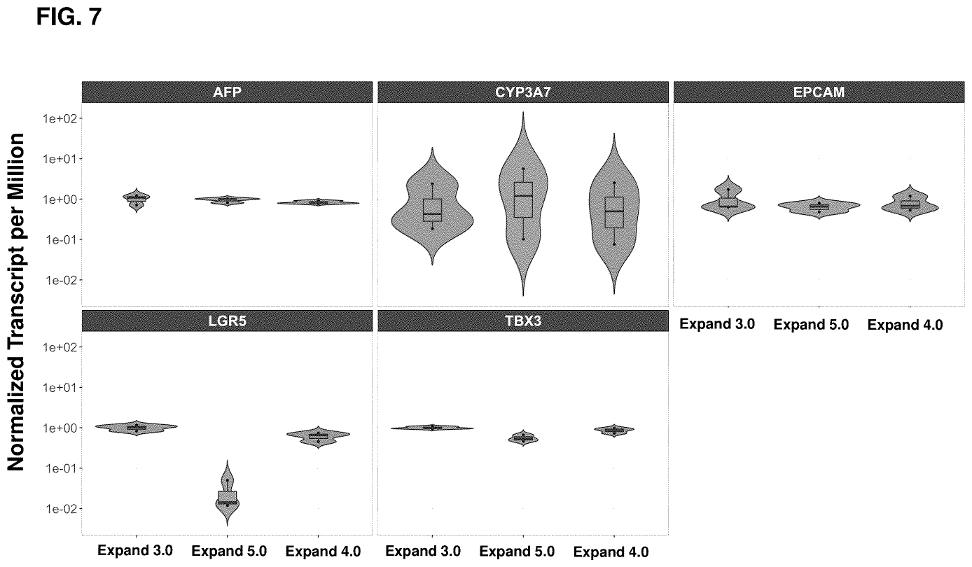

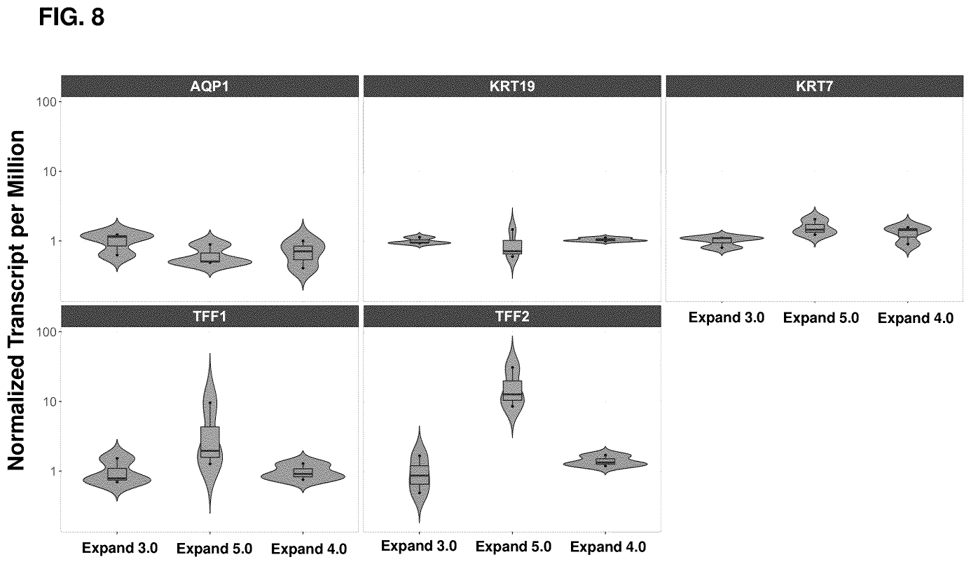

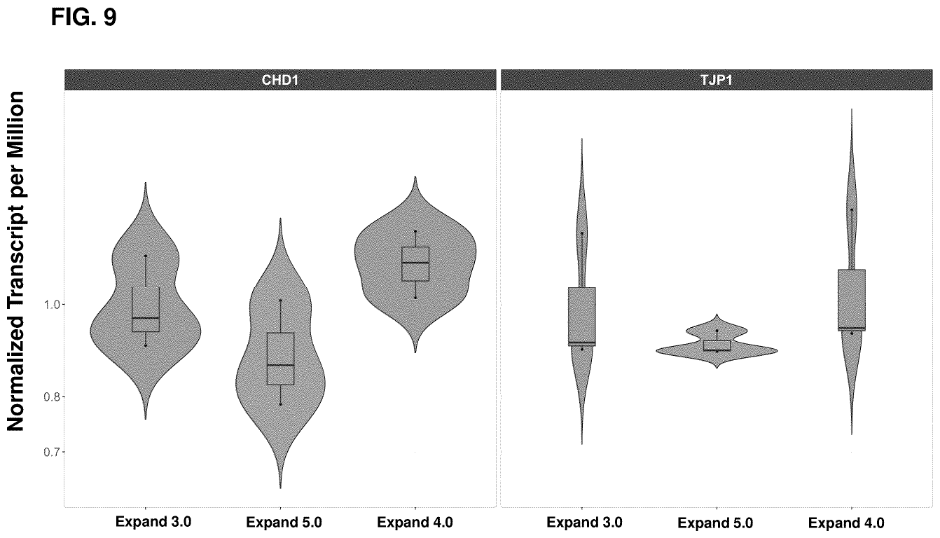

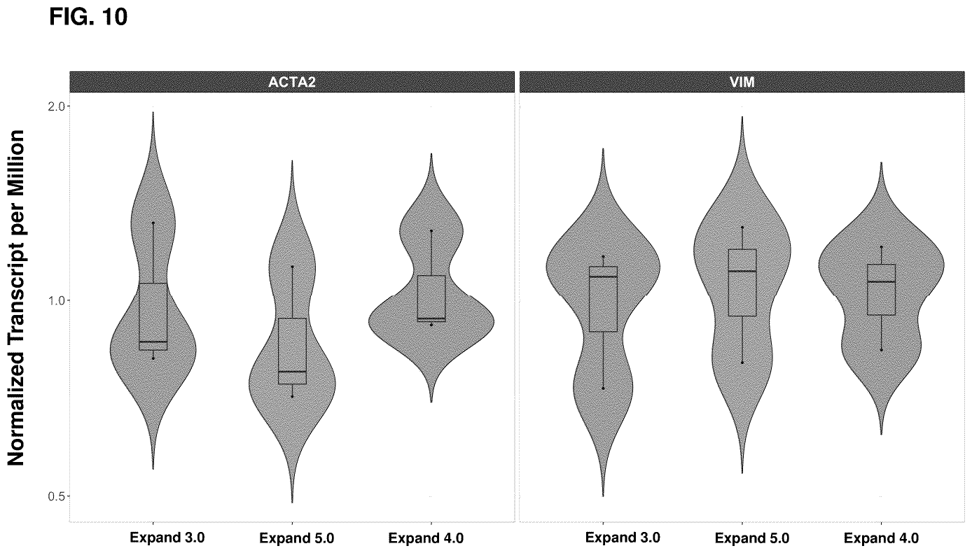

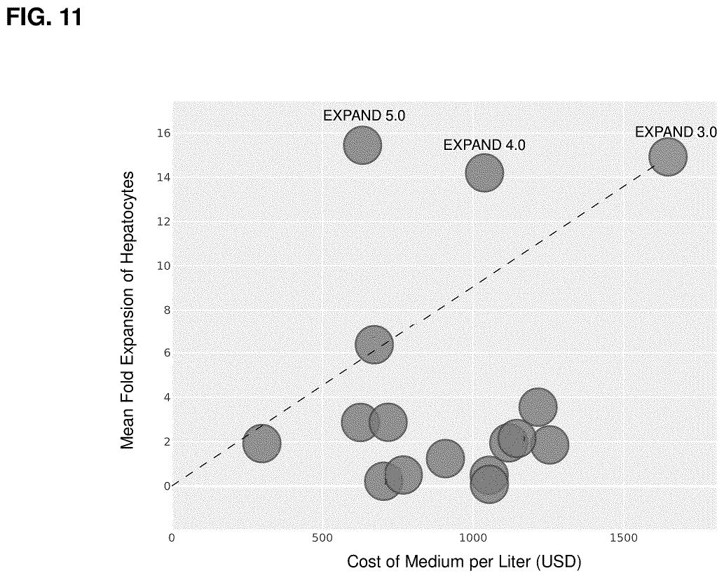

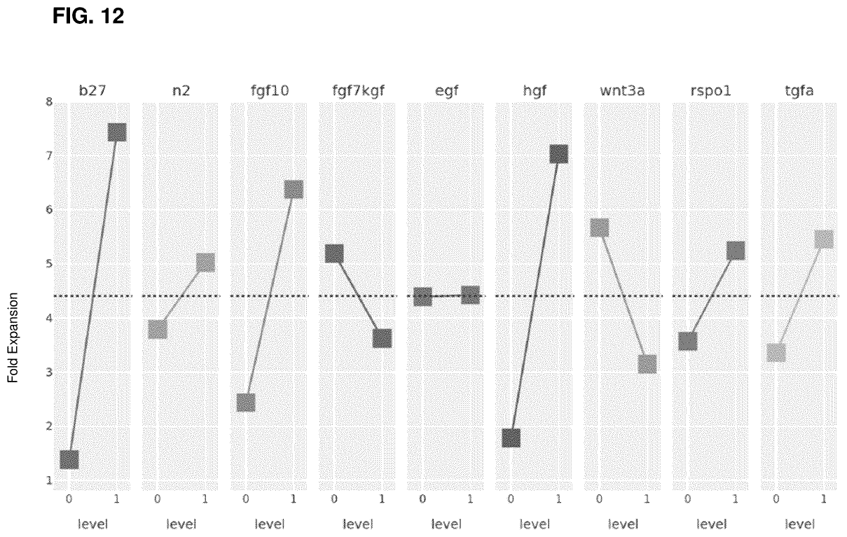

Expression levels of various biomarkers were then evaluated via RNA-seq analysis. Expression levels of hepatocyte lineage markers including albumin (ALB), cytochrome P450 1A2 (CYP1A2), cytochrome P450 3A4 (CYP3A4), hepatocyte nuclear factor 4 alpha (HNF4A), nuclear receptor subfamily 1 group I member 2 (NR112), and serpin family A member 1 (SERPINA1) were compared in hepatocytes that were expanded in each expansion medium (FIG. 4). The expression levels of urea cycle biomarkers including arginase 1 (ARG1), argininosuccinate lyase (ASL), argininosuccinate synthase 1 (ASS1), carbamoyl phosphate synthetase 1 (CPS1), N-acetylglutamate synthase (NAGS), and ornithine transcarbamylase (OTC) were also measured for the expanded hepatocytes (FIG. 5). Moreover, expression levels of biomarkers for cell polarity (FIG. 6), hepatocyte progenitor cells (FIG. 7), cholangiocytes (FIG. 8), epithelial cells (FIG. 9), and mesenchymal cell fate (FIG. 10) were similarly evaluated. These data demonstrate that the newly developed Expand 4.0 and Expand 5.0 expansion media effectively expands hepatocytes, maintains hepatocyte cell identity, and provides a more cost-effective formulation with fewer components compared to the Expand 3.0 expansion medium. The cost benefit and fold expansion of these three medium formulations, among others, is shown in FIG. 11. In addition to cost benefit and fold expansion analysis across various medium formulations, the presence of individual additives and their contribution to hepatocyte expansion was modeled using an ordinary least squares (OLS) model. Most additives showed positive coefficients, which indicates that the addition of these components can increase fold expansion of hepatocytes (FIG. 12). Interestingly, FGF7/KGF and Wnt3a exhibited negative coefficients, which indicates that the presence of these components can decrease fold expansion of hepatocytes (FIG. 12).

### Example 2. Serum Replacement Component and Non-Essential Amino Acids Each Improve Hepatocyte Expansion

This example describes adding (i) a serum replacement component and (ii) specific non-essential amino acids for improved hepatocyte expansion.

In addition to developing the Expand 4.0 and Expand 5.0 expansion medium formulations shown in Example 1, other expansion medium formulations were developed to assess the contribution of various additives to hepatocyte expansion. These formulations were based on the Expand 1.0 expansion medium shown in Table 2.

TABLE 2
Expand 1.0 expansion medium

| | Components | EXPAND 1.0 Cocktail | |
|---|---|---|---|
| 1 | DMEM based media | Base Medium | |
| 2 | Penicillin/Streptomycin | 1% | |
| 3 | L-glutamine alternative | 1% | |
| 4 | HEPES | 10 | mM |
| 5 | B27 Supplement | 1X | |
| 6 | N2 Supplement | 1X | |
| 7 | Rspo-1 | 100 | ng/mL |
| 8 | Wnt3a | 50 | ng/mL |
| 9 | N-acetylcysteine | 1.25 | mM |
| 10 | Nicotinamide | 100 | ng/mL |
| 11 | Recombinant EGF | 50 | ng/mL |
| 12 | Recombinant TGF-alpha | 20 | ng/mL |
| 13 | Human FGF-7 | 50 | ng/mL |
| 14 | Human FGF-10 | 50 | ng/mL |
| 15 | Human HGF | 25 | ng/mL |
| 16 | A83-01 | 2 | μM |
| 17 | ROCK inhibitor (Y-27632) | 10 | μM |

| | | |
|---|---|---|
| 18 | KOSR | Not Added |
| 19 | NEAA (100X) | Not added |
| | Hypoxic culture conditions? | No |

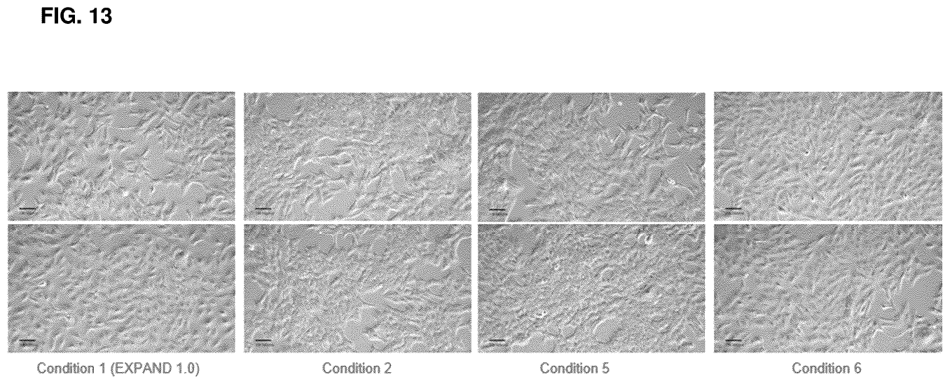

The additional medium conditions and resulting hepatocyte expansion yield following thirteen days of culture after seeding are described in Table 3. Brightfield images of hepatocyte morphology following thirteen days of culture are shown in FIG. 13.

TABLE 3
Cell culture formulation conditions and cell expansion

| | Normoxia | ROCK | Amino Acids | | Fold |
|---|---|---|---|---|---|
| Condition | (20-21% O₂) | Inhibitor | (NEAA) | KOSR | Expansion |
| Expand 1.0 | + | 10 μM | Not Added | Not Added | 8.78-fold |
| Condition 2 | + | Not Added | Not Added | Not Added | 27.7-fold |
| Condition 5 | + | Not Added | 1X | 1% | 37.5-fold |
| Condition 6 | + | Not Added | 1X | 10% | 36.5-fold |

While the Expand 1.0 expansion medium had trace amino acids present in the basal cell culture medium, adding the NEAA supplement increased the concentration of amino acids glycine, alanine, asparagine, aspartic acid, glutamic acid, proline, and serine as shown in Table 4.

TABLE 4
Amino acid supplementation in Expand medium formulations

| | Concentration in Basal | Concentration with |
|---|---|---|
| Amino Acids | Cell Culture Medium (mM) | Supplementation (mM) |
| Glycine | 0.25 | 0.35 |
| Alanine | 0.049 | 0.149 |
| Asparagine | 0.05 | 0.15 |
| Aspartic Acid | 0.05 | 0.15 |
| Proline | 0.15 | 0.25 |
| Serine | 0.25 | 0.35 |

In addition to improved fold expansion relative to the Expand 1.0 expansion medium, the hepatocytes cultured in the additional medium conditions shown in Table 4 reached confluence after fewer days of culturing and had faster doubling time. For instance, cell culture conditions following culture in medium Condition 5 and the Expand 1.0 expansion medium are compared in Table 5.

TABLE 5
Comparison of hepatocyte expansion in different cell culture medium conditions

| | Days to reach | | | | |
|---|---|---|---|---|---|
| | confluency/ | Cell yield at | Absolute | Number of | Doubling |
| Expand | near | confluency/near | Fold | population | Time |
| formulation | confluency | confluency | Expansion | doublings | (Days) |
| Expand 1.0 | 23 | 1.5 million cells | 15-fold | ~6 | 3.8 days |
| | | | | (1.6 million) | |
| Condition 5 | 13 | 3.75 million cells | 37.5-fold | ~7 | 1.85 days |
| | | | | (3.2 million) | |

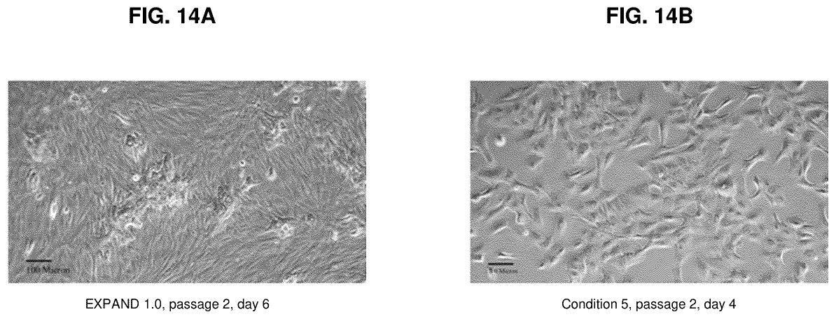

In addition to the differences in expansion, the morphology of the cells cultured in the Expand 1.0 and Condition 5 cell culture medium formulations differed. After two passages, the hepatocytes cultured in the Expand 1.0 expansion medium were heterogenous and had a mesenchymal morphology (FIG. 14A), in contrast to the hepatocytes cultured in the Condition 5 cell culture medium, which were homogenous and maintained hepatocyte morphology (FIG. 14B).

These results demonstrate that omitting a ROCK inhibitor, supplementing the amino acids shown in Table 4 (i.e., glycine, alanine, asparagine, aspartic acid, proline, and serine), and adding a serum replacement component to the cell culture medium improves hepatocyte expansion.

### Example 3. A Gradient of Serum Replacement Component Gradient Enhances Hepatocyte Expansion

This example describes the effect of serum replacement component concentration on hepatocyte culture.

To further evaluate potential benefits from culturing hepatocytes upon addition of a serum replacement component to the cell culture medium, the concentrations of the serum replacement component were further modulated. Since there were no observed improvements in expansion when hepatocytes were cultured in an expansion medium that contained 1% or 10% serum replacement component as shown in Example 2, a graded increase of serum replacement component over the course of the culture was assessed. PHHs were seeded and expanded in either the Expand 1.0 expansion medium or the Condition 5 expansion medium, as described in Example 2, in which the Condition 5 medium was formulated with 1%, 5%, or 10% volumetric concentrations of serum replacement component such that the volume of serum replacement component increased during the 14-day culture as the medium was replaced. The medium conditions and resulting hepatocyte expansion are summarized in Table 6.

TABLE 6
Hepatocyte expansion in static or gradient
serum replacement component conditions

| | | Serum | |
|---|---|---|---|
| | | Replacement | Hepatocyte |
| Media Formulation | Culture Day | Component | Expansion |
| Expand 1.0 | Day 1-Day 6 | Not Added | 4.5-fold |
| (No KOSR) | Day 7-Day 8 | | |
| | Day 9-Day 14 | | |
| Condition 5 | Day 1-Day 6 | 1% | 48-fold |
| (Static KOSR) | Day 7-Day 8 | | |
| | Day 9-Day 14 | | |
| Condition 5 | Day 1-Day 6 | 1% | 79-fold |
| (Increasing KOSR) | Day 7-Day 8 | 5% | |
| | Day 9-Day 14 | 10% | |

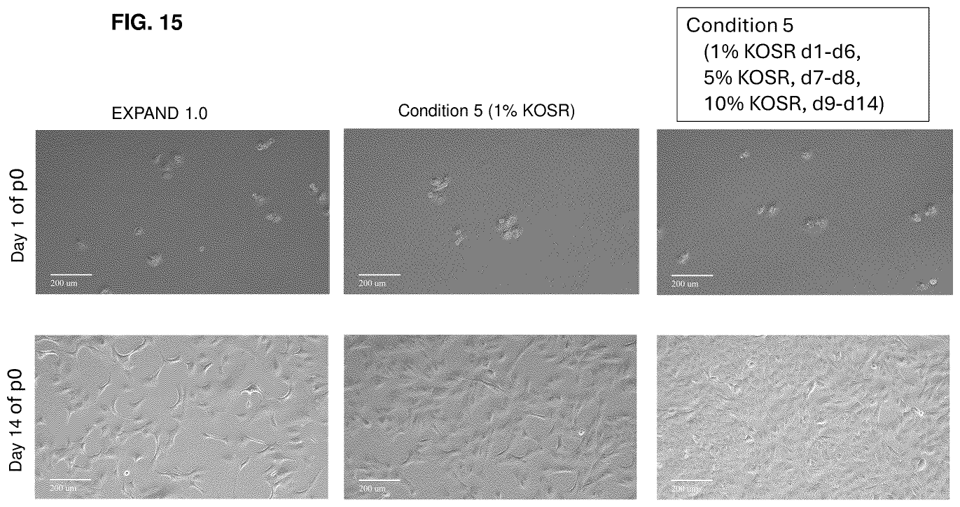

Surprisingly, gradually increasing the serum replacement component added to the expansion medium over the course of culturing hepatocytes led to a substantial increase in the yield of expanded hepatocytes with no effect on cell morphology (FIG. 15).

### Example 4. Evaluating Cell Expansion Method in a Three-Dimensional Culture Format

This example describes expansion of hepatocytes when cultured in a three-dimensional culture format.

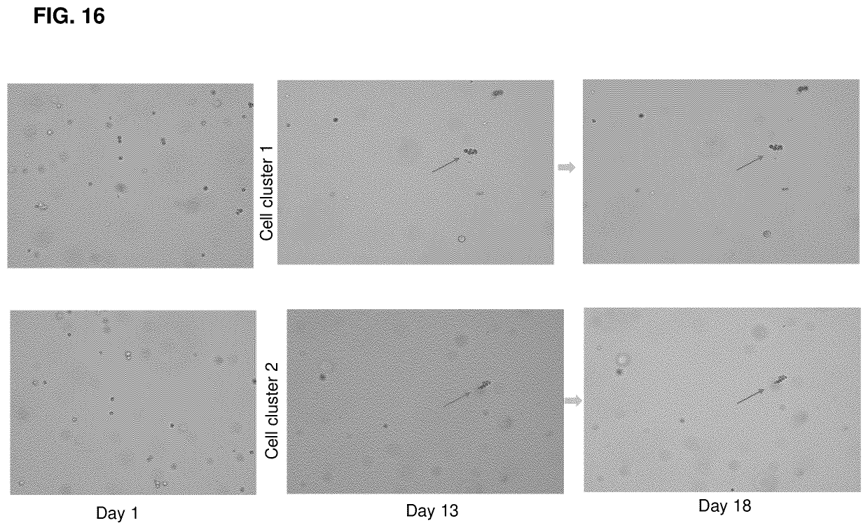

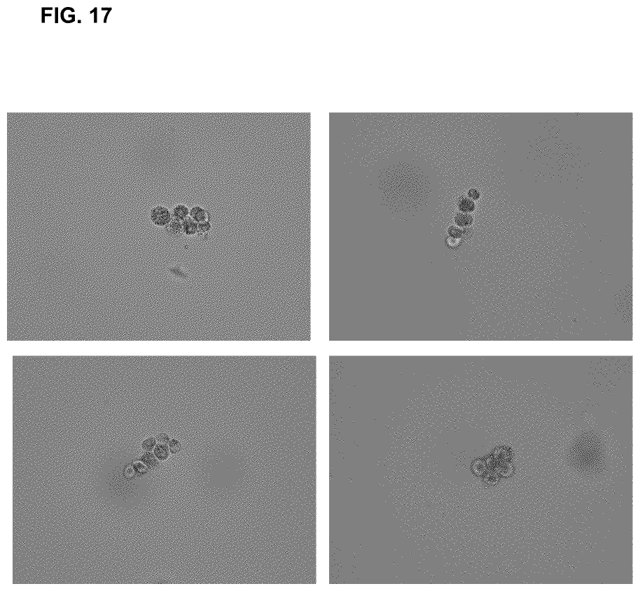

PHHs (25,000 cells) were resuspended in an ECM-hydrogel mixture (CULTREX® Basement Membrane Extract) at a ratio of 3:1 hydrogel mixture to expansion medium (Expand 1.0 medium, as described in Example 2) and plated at about 300 μL per well in a 24-well plate. After solidification of the hydrogel, 500 mL of Expand 1.0 expansion medium were added to each well, and the medium was replaced every two days. After 18 days of culture, no growth or expansion was observed in the formed cell clusters (FIGS. 16 and 17).

### Example 5. Evaluation of Hepatocyte Cultures on Extracellular Matrix Substrates

This example describes culturing hepatocytes in expansion medium on different extracellular matrix substrates.

Six-well plates were coated with different concentrations of various extracellular matrix substrates for expansion of three different lots of PHHs. The plates were coated in laminin-521 or vitronectin at concentrations of 0.125 μg/cm², 0.25 μg/cm², and 0.5 μg/cm². Additionally, some plates were coated in 75 μL/cm² of CELLstart™ xeno-free substrate (Gibco), in which three volumetric dilutions of CELLstart™ were tested: 1:50, 1:100, and 1:200. The three hepatocyte lots were seeded on each substrate condition at seeding densities of 325 cells/cm², 650 cells/cm², or 1,300 cells/cm² and cultured in Expand 4.0 expansion medium for 13 days.

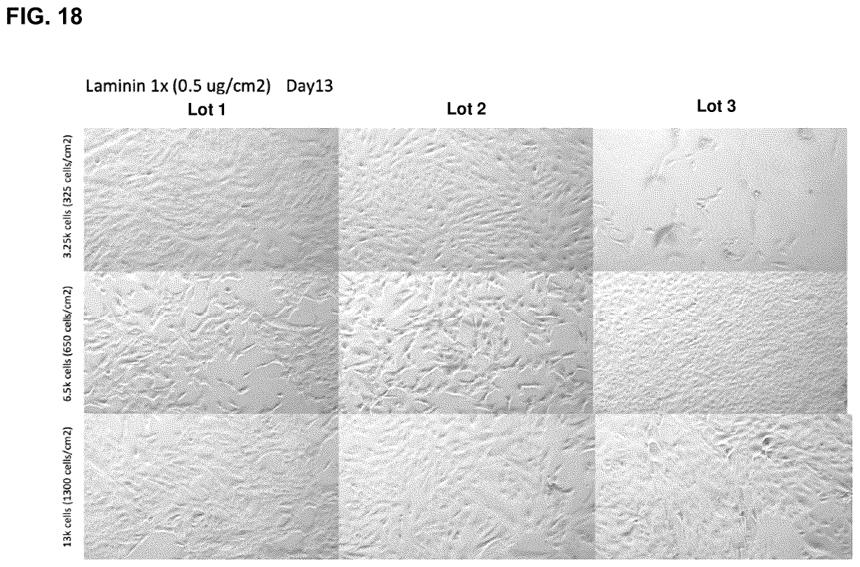

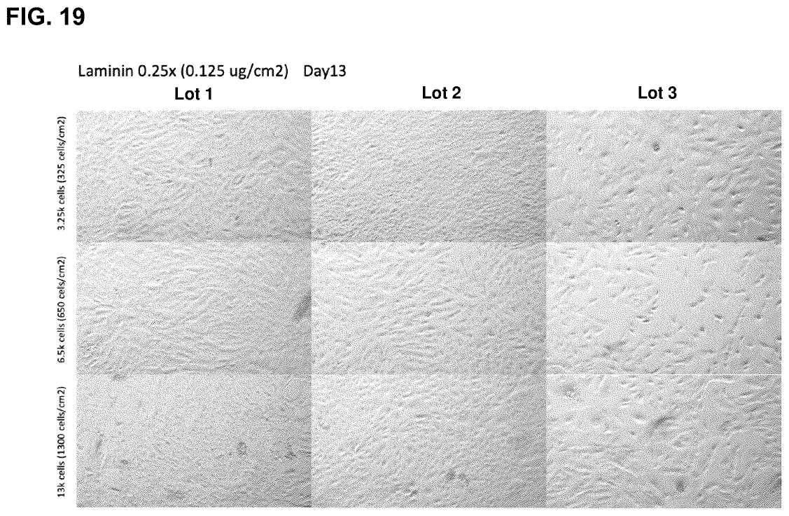

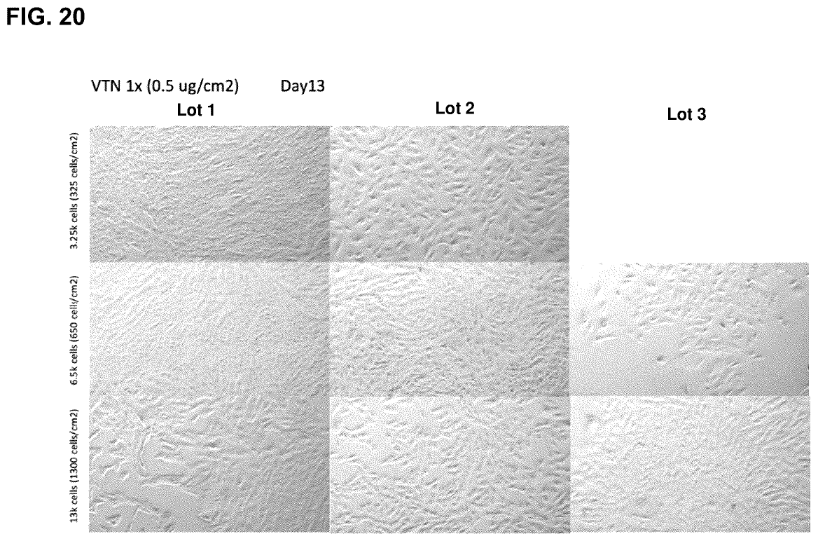

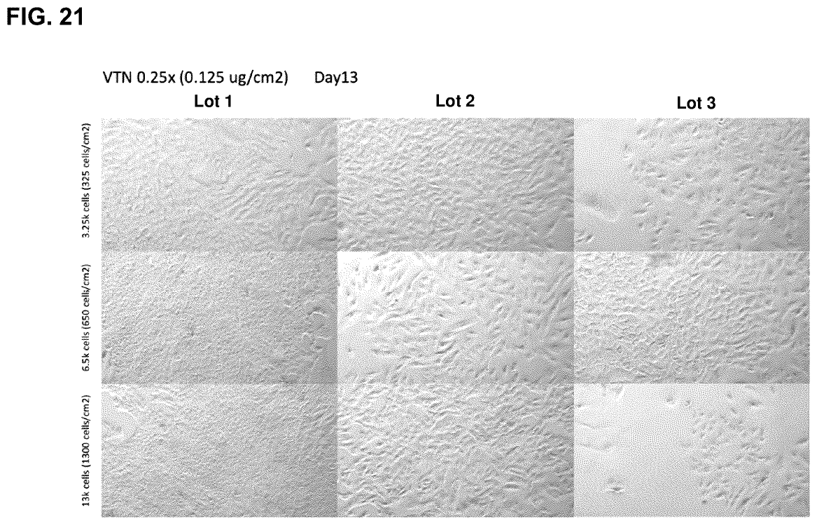

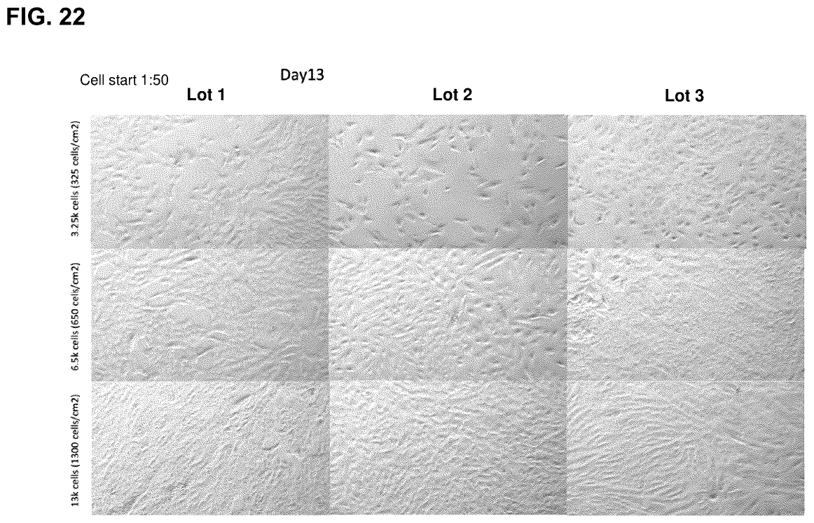

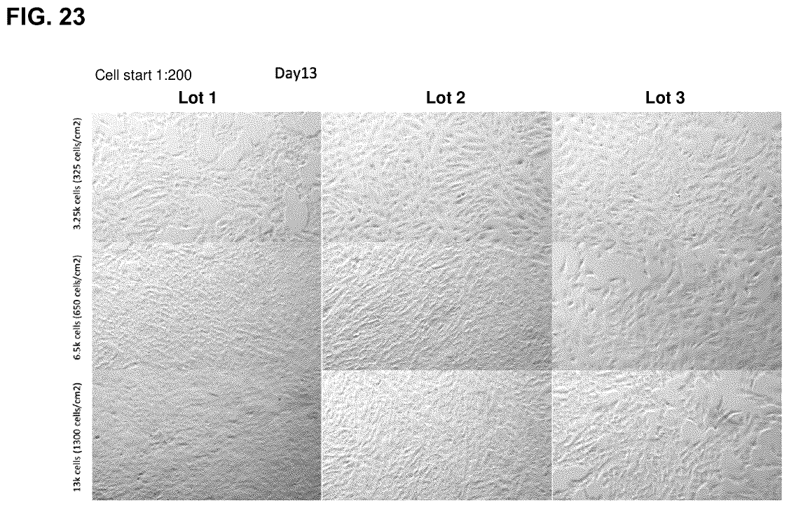

Cultures that were seeded at higher densities reached confluence sooner than cultures seeded at lower densities. After 13 days of expansion, cell morphology was confirmed to be comparable across all conditions (FIGS. 18-23).

### Example 6. Two Passage Expansion of Primary Human Hepatocytes

This example describes a high-yield 24-day expansion of PHHs using the Expand 4.0 expansion medium as described in Example 1.

A cryopreserved population of PHHs were thawed and plated at a density of 667 viable cells/cm² in the Expand 4.0 expansion medium in T75 cell culture flasks coated in 0.125 μg/cm² laminin-521. These plated cells (passage 0 (P0)) were cultured for 13 days at 37° C. under 5% oxygen hypoxic atmosphere, and the expansion medium was replaced every 48 hours. After 13 days, the cells were a confluent monolayer. The cells were then dissociated and seeded at a density of 1,333 viable cells/cm² in fresh expansion medium (passage 1 (P1)) and were cultured for an additional 11 days. The cells expanded by nearly 100-fold after expansion step P0, and collectively, P0 and P1 expansion in Expand 4.0 expansion medium yielded greater than 1,000-fold expansion of PHHs.

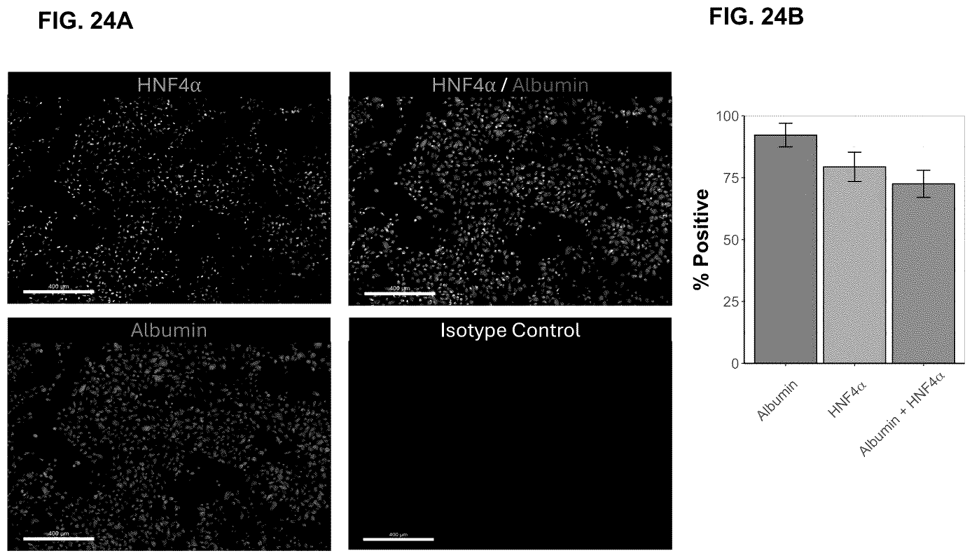

Immunofluorescence staining confirmed that the expanded hepatocytes maintained expression of albumin and HNFα (FIGS. 24A and 24B), demonstrating that hepatocyte cell identity was preserved during expansion with Expand 4.0 expansion medium.

### Example 7. Modulating Concentrations of Growth Factor and Metabolite Additives in Expansion Medium

This example describes the expansion of hepatocytes by varying the added amount of growth factors and other metabolites in the Expand 4.0 expansion medium as described in Example 1. To identify additional formulations of an effective expansion medium, the Expand 4.0 expansion medium was used as a base formulation, except the starting modeled Rspo-1 concentration was increased, and the concentrations of different additives were assessed. The described analyses focus primarily on varying the concentrations of FGF-10 and Rspo-1, which are the two most expensive growth factors used in the Expand 4.0 expansion medium, as well as N2 and B27 supplements, which are widely utilized supplements for cell culturing. Thus, these additional formulations represent functional expansion media that are cost-effective and are more likely to withstand lot-to-lot variability of commercially available reagents.

After culturing hepatocytes in the different medium formulations that included varying concentrations of FGF-10, Rspo-1, N2, and B27 supplements, the fold expansion and viability results were used to generate a model to examine the effects of these additives on cell culture quality. The fold expansion and viability responses were modeled as a function of the input concentrations in standardized units ranging from −1.94 to 1.94 for the four representative factors as shown in Table 7 to maintain each predictor on an identical scale and avoid any predictor dominating the regression solver based on larger absolute units.

TABLE 7
Modeled additive reduction

| | 1.94 | | | | | | | | | |
|---|---|---|---|---|---|---|---|---|---|---|
| Component | (Starting Concentration) | | 1 | | 0 | | −1 | | −1.94 | |
| Human FGF-10 | 50 | ng/mL | 39 | ng/mL | 28 | ng/mL | 15 | ng/mL | 5 | ng/mL |
| Rspo-1 | 100 | ng/mL | 79 | ng/mL | 55 | ng/mL | 20 | ng/mL | 10 | ng/mL |
| N2 Supplement | 10 | mL/L | 8 | mL/L | 6 | mL/L | 2 | mL/L | 1 | mL/L |
| B27 Supplement | 20 | mL/L | 16 | mL/L | 11 | mL/L | 4 | mL/L | 2 | mL/L |

Each model included main effects, second order interactions, and second order polynomial terms. The model was fit and then reduced by trimming terms with high p values (p>0.15). An appropriate fit was ensured by comparing the adjusted coefficient of determination (R² adjusted) values between the full and reduced model, and the Nelder-Mead algorithm was used to solve for the maximum on the linear model surface.

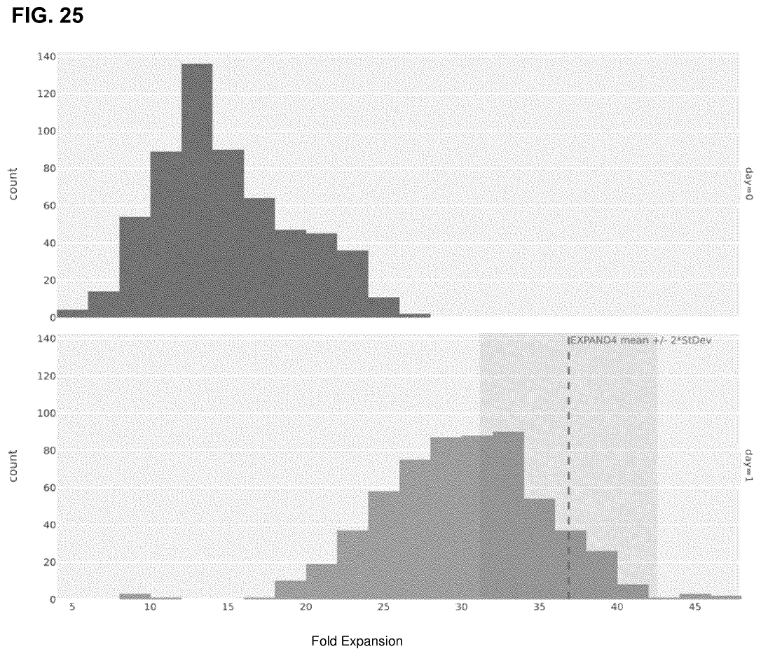

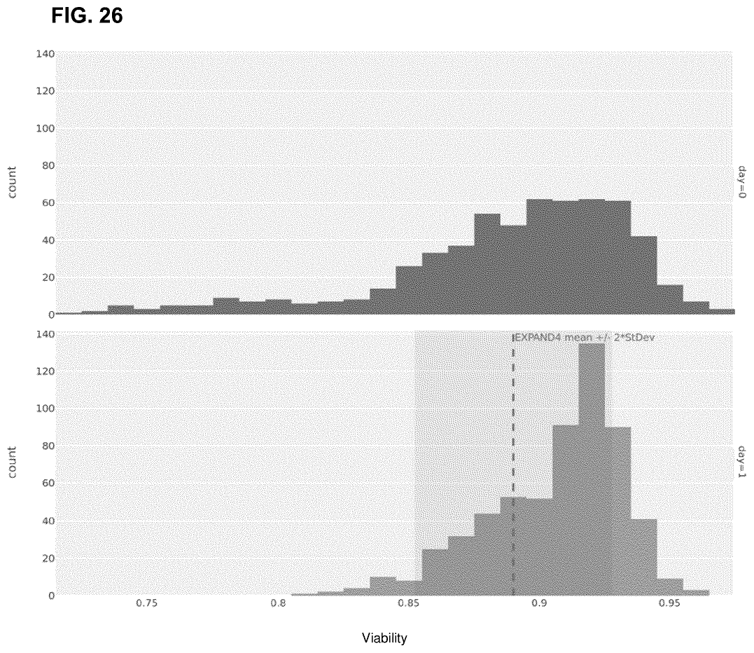

First, the resulting modeled fold expansion appeared mostly normal and unimodal but appeared to have a day-to-day effect in which day 1 produced approximately 2× higher fold expansion relative to day 0. The day 1 fold expansion aligned with the mean of the standard Expand 4.0 media, suggesting that the day 1 values were not aberrantly high (FIG. 25). The modeled viability response showed more alignment of the distributions between days 0 and 1, suggesting that the fold expansion day-to-day effect was independent of viability (FIG. 26).

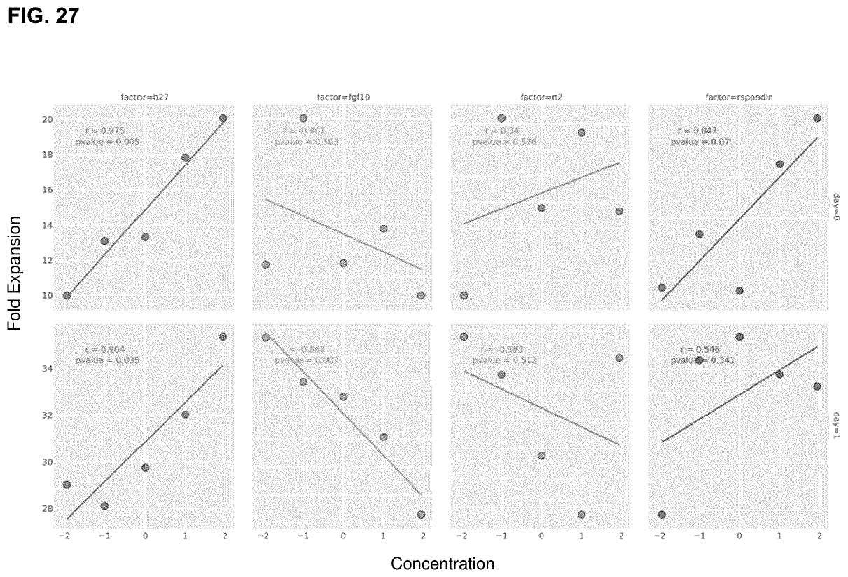

The trend consistency across the two days of experiments were evaluated by comparing OLS trendlines through the means of each concentration by day (FIG. 27). For B27 supplementation, both days produced a trendline with a coefficient with a low p-value (p<0.05) and a Pearson correlation coefficient (Pearson r)>0.9, suggesting that B27 concentration positively associated with fold expansion on both days. For FGF-10, the trend was negative on both days (r<−0.4) but only day 1 was significant (p=0.007), suggesting a mostly negative main effect. In contrast, the trend for N2 supplementation was not significant on either day, suggesting that there was no observed effect within the tested concentration range. Lastly, the Rspo-1 trend was positive (r>0.5) but not significant for either day.

### Example 8. In Vitro Maturation of Expanded Human Hepatocytes on the Surface of a Cell Culture Vessel

This example describes hepatocyte functionality and identity following expansion in Expand 3.0 expansion medium in addition to hepatocyte maturation or hepatocyte maintenance in an in vitro cell culture format, in which the hepatocytes were cultured on the surface of a cell culture vessel. In particular, expanded and/or matured hepatocytes were evaluated for activity of cytochrome P450 3A4 (CYP3A4) enzyme activity, which is a robust biomarker of hepatocyte maturity as well as expression of other cell biomarkers.

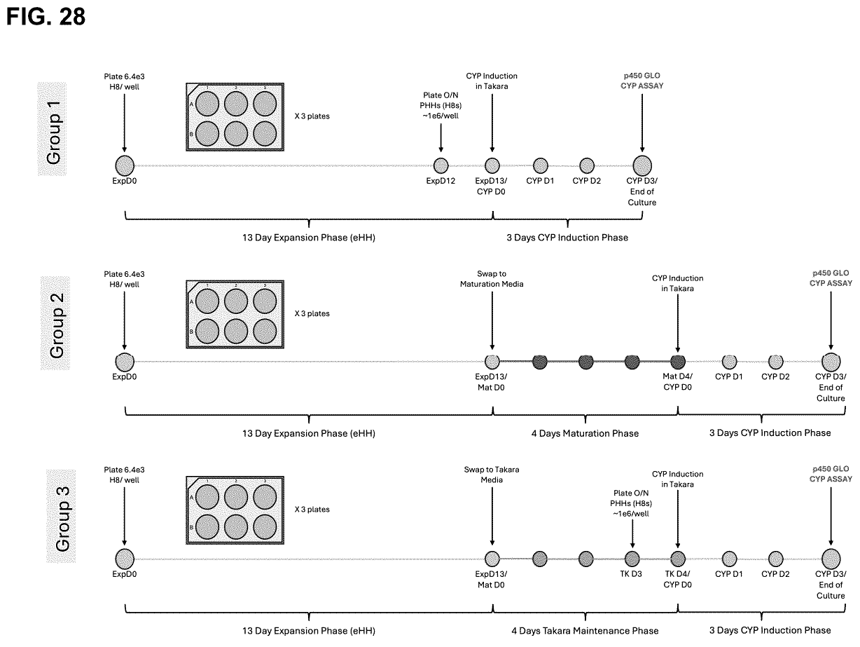

The in vitro expansion and subsequent maturation were performed in three experimental groups as outlined in FIG. 28. PHHs were thawed and seeded at a density of 6.4×10³ cells per well in a 6-well plate and then expanded for 13 days in Expand 3.0 expansion medium as described in Example 1. One group of expanded hepatocytes underwent CYP induction for three days immediately after expansion (Group 1). The expanded hepatocytes were then matured for 4 days in the 18S+3UC maturation medium (Table 8; Group 2) or were maintained for 4 days in Takara Cellartis POWER™ Primary HEP medium (Group 3) prior to CYP induction in Takara Cellartis POWER™ Primary HEP medium for three days. After CYP induction, activity of CYP3A4 was measured using the Promega P450-GLO™ CYP3A4 Kit according to manufacturer specifications. In each experimental group, cells were treated with either dimethyl sulfoxide (DMSO) to inhibit CYP3A4 activity or rifampicin as a positive control to induce CYP3A4 activity. Additionally, a population of PHHs plated overnight in Expand 3.0 expansion medium and a population of PHHs that were plated overnight in Takara Cellartis POWER™ Primary HEP medium were used as additional controls.

TABLE 8
17S + 3UC maturation medium composition

| | |
|---|---|
| Components | Concentration |

| 1 | Lonza HCM Medium | Base Medium | |
|---|---|---|---|
| 2 | Penicillin/Streptomycin (100X) | 1X | |
| 3 | HEPES (100X) | 1X | |
| 4 | GLUTAMAX ™ (100X) | 1X | |
| 5 | Insulin Transferrin Selenium (ITS) (100X) | 1X | |
| 6 | Compound E | 0.1 | μM |
| 7 | Erlotinib HCl | 2 | μM |
| 8 | Oncostatin M | 20 | ng/ml |
| 9 | Dexamethasone | 10 | μM |
| 10 | Hydrocortisone (Lonza CC-4182, 1000X) | 1X | |
| 11 | Vitamin C | 0.5 | mM |
| 12 | Vitamin K2 | 10 | μM |
| 13 | Lithocholic Acid | 10 | μM |
| 14 | Urso deoxycholic Acid | 25 | μM |
| 15 | 8 Bromo cAMP | 1 | mM |
| 16 | Forskolin | 5 | μM |
| 17 | Cholesterol (250X) | 1X | |
| 18 | T3 | 3 | μM |
| 19 | NEAA (100X) | 6.25X | |
| 20 | EAA (50X) | 12.5X | |
| 21 | Glucagon | 100 | nM |

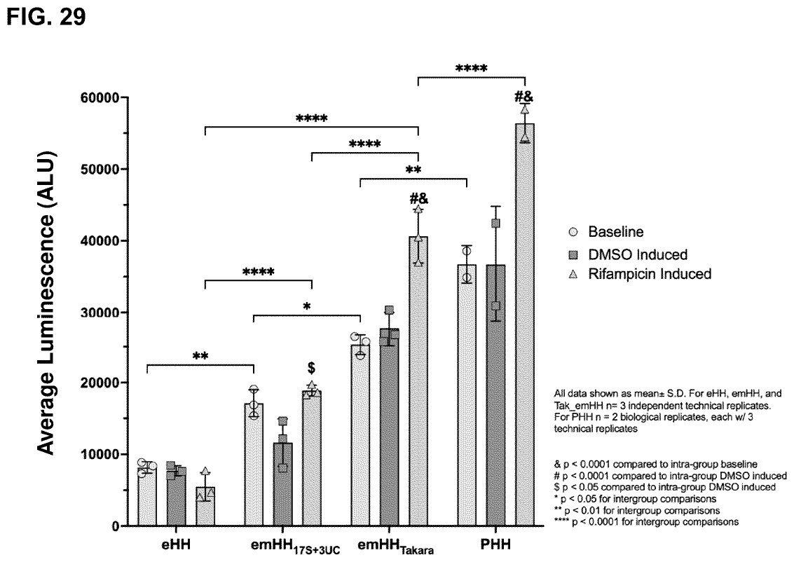

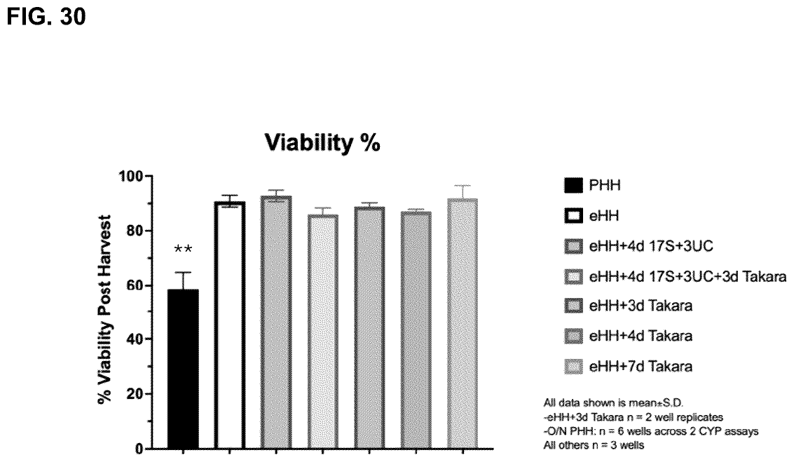

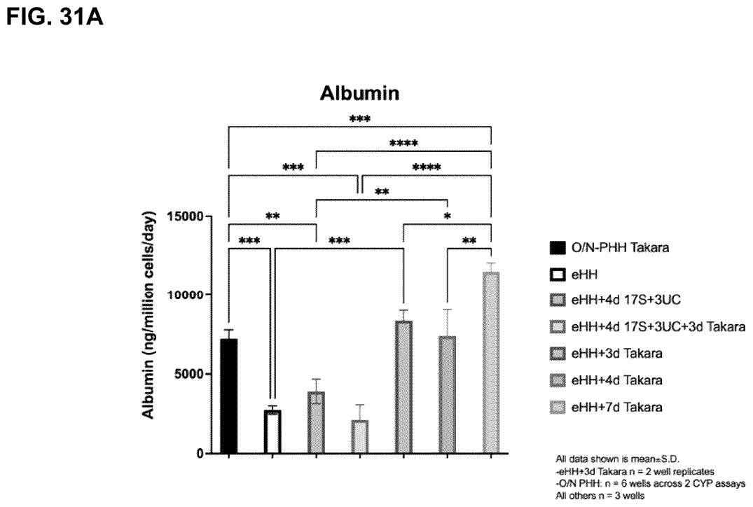

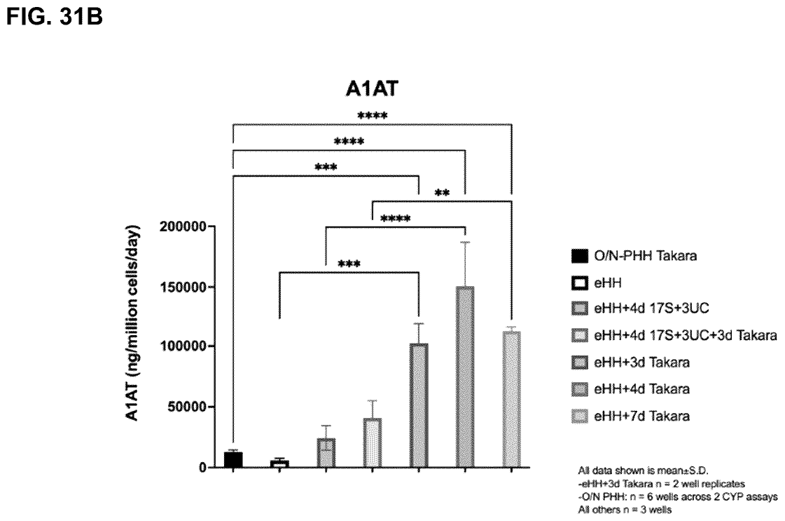

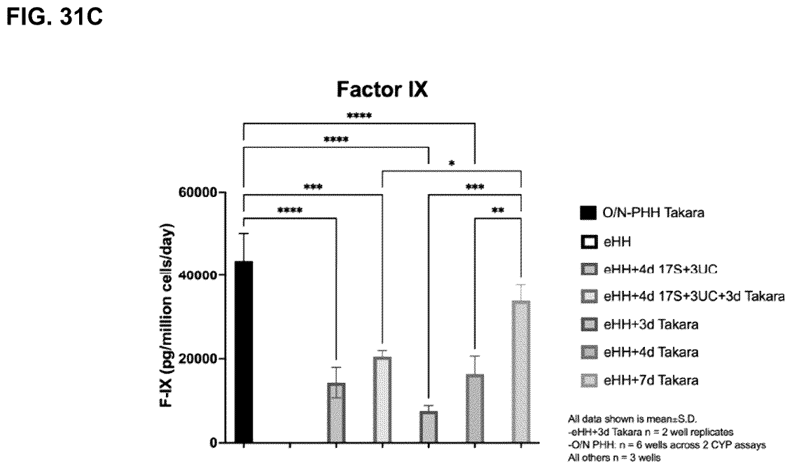

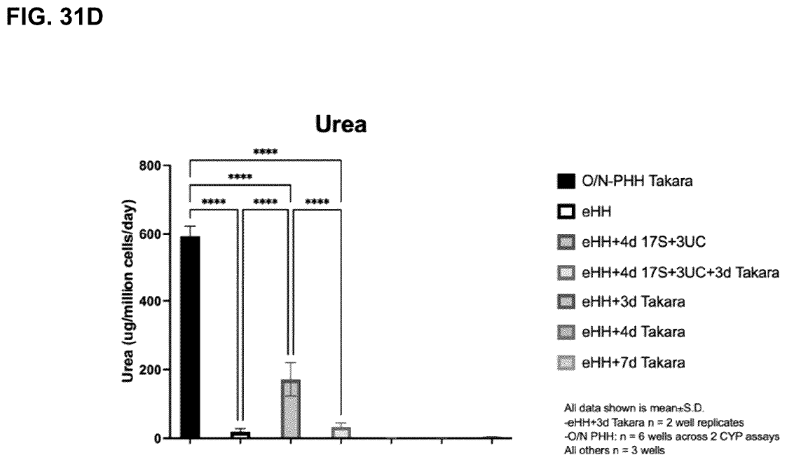

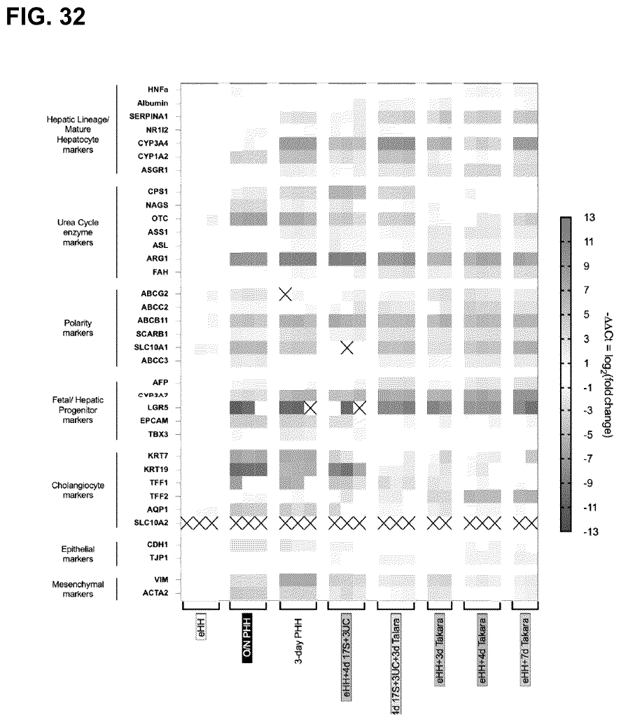

The CYP activity of the PHHs and expanded and/or matured hepatocytes is shown in FIG. 29. Cell viability over the course of the experiment as measured by trypan blue staining is shown in FIG. 30. Samples of the cell culture medium were collected over the course of the experiment to measure levels of secreted albumin (FIG. 31A), alpha-1 antitrypsin (FIG. 31B), factor IX (FIG. 31C), and urea (FIG. 31D) of the cells cultured in each condition. Moreover, gene expression analysis via RNA-seq was performed to measure mRNA expression levels of biomarkers indicative of hepatocytes, urea cycle enzymes, cell polarity, hepatocyte progenitors, cholangiocytes, epithelial cells, and mesenchymal cells across each condition (FIG. 32).

### Example 9. In Vitro Maturation of Induced Pluripotent Stem Cell-Derived Hepatocytes on the Surface of a Cell Culture Vessel

This example describes hepatocyte functionality and identity of iPSC-derived hepatocytes following maturation in various maturation media in an in vitro format, in which the hepatocytes were cultured on the surface of a cell culture vessel.

Commercially available iPSC-derived hepatocytes (iHEPs) were plated and cultured in plating medium for 5 days and then cultured in iHEP maintenance medium (Table 9) for 2 days. After this maintenance step, the iHEPs were either cultured for 4 additional days in iHEP maintenance medium, or they were matured for 4 days in 17S+3UC maturation medium, as described in Table 8 in Example 8.

TABLE 9
iHEP maintenance medium composition

| | Component | Volume | |
|---|---|---|---|
| 1 | RPMI 1640 Medium (Base) | 72 | mL |
| 2 | B27 Supplement (50X) | 1.5 | mL |
| 3 | Dexamenthasone (5 mM) | 1.5 | μL |
| 4 | Gentamicin (50 mg/mL) | 37.5 | μL |
| 5 | iCell Hepatocytes 2.0 Medium Supplement | 1.5 | mL |

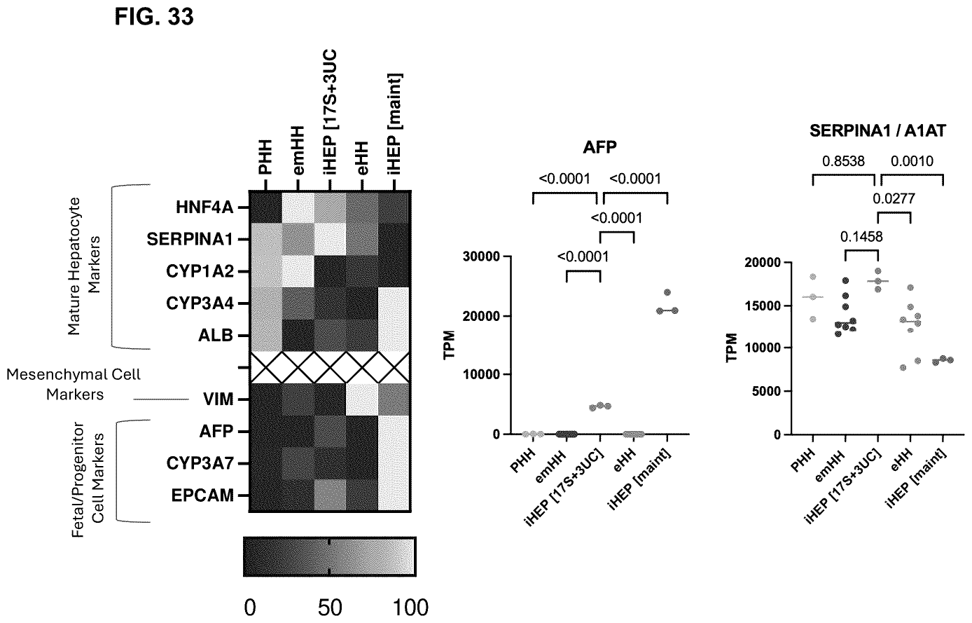

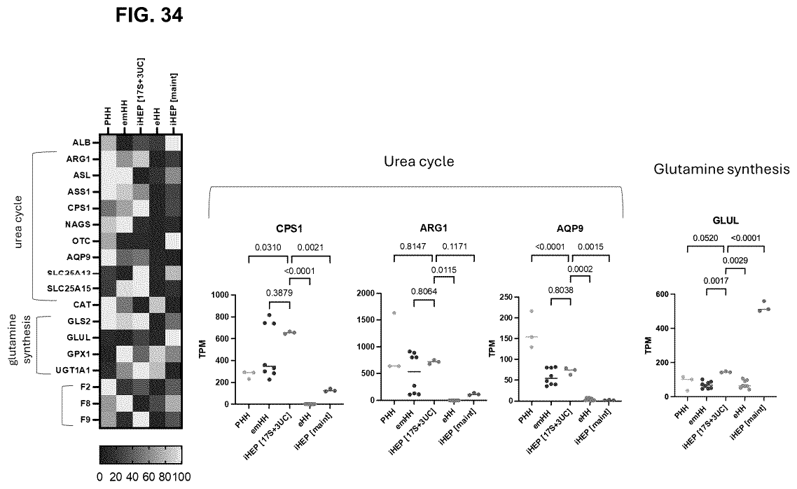

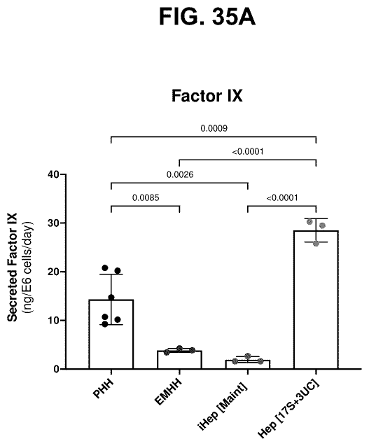

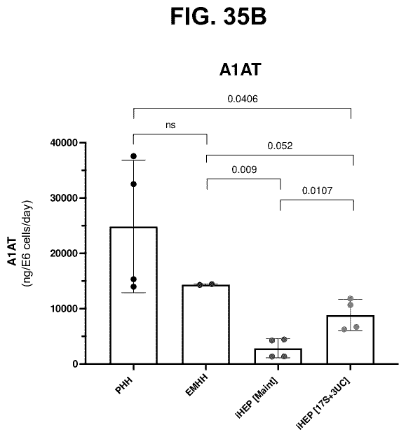

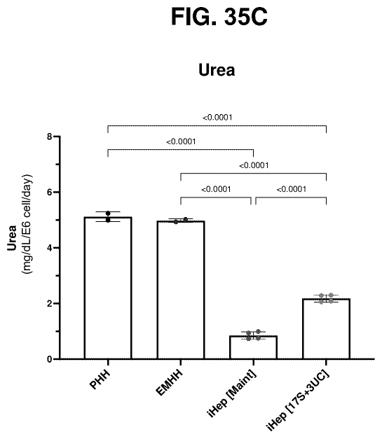

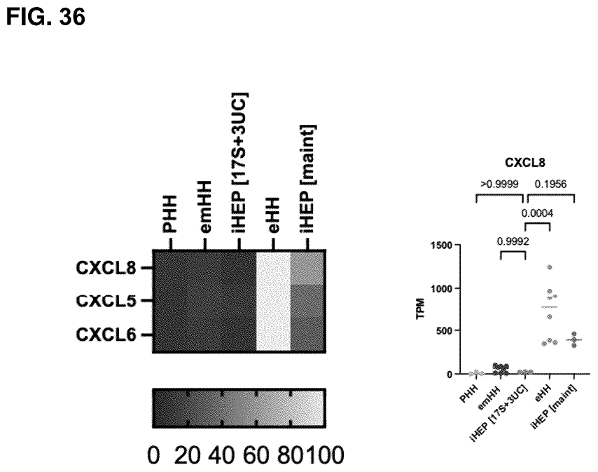

Expression levels of biomarkers of hepatocyte identity (FIG. 33) and ammonia clearance (FIG. 34) were evaluated by RNA-seq and compared to PHHs and PHHs that were expanded and/or matured as described in Example 4. Further, samples of cell culture media were taken, and expression levels of secreted biomarkers factor IX (FIG. 35A), alpha-1 antitrypsin (FIG. 35B), and urea (FIG. 35C) were determined for each cell culture condition. These data confirm that the matured iHEPs maintain hepatocyte identity. Additional RNA-seq analysis demonstrated that the matured iHEPs, expanded and matured hepatocytes, and PHHs had lower expression levels of cytokines CXCL5, CXCL6, and CXCL8 compared to expanded hepatocytes or maintained iHEPs (FIG. 36).

### Example 10. In Vitro Maturation of Expanded Hepatocyte Seeds

This example describes hepatocyte functionality following expansion in Expand 3.0 expansion medium and maturing the hepatocytes in vitro as seeds that include expanded hepatocytes and fibroblasts.

Cryopreserved populations of normal human dermal fibroblasts (NHDF) and expanded human hepatocytes were thawed and combined at a ratio of 2:1 NHDF to expanded human hepatocytes. Seeds (50,000) were aggregated in xeno-free aggregation medium (DMEM+4.5 g/L D-glucose+L-glutamine; 100 g/mL penicillin and streptomycin; 10% (v/v) human serum; 70 ng/ml glucagon; 50 ng/ml dexamethasone) or LIFENET HEALTH® Human Hepatocyte Medium in vertical wheel bioreactors (VWBs). After 22 hours, the aggregates were washed in a Rotea centrifugation system (day 0) and were then cultured for four days under different conditions summarized in Table 10 below.

TABLE 10
Aggregation conditions

| Group | Aggregation Medium | Culture Medium | Rotor Speed |
|---|---|---|---|
| 1 | Xeno-free aggregation | LIFENET HEALTH ® Human Hepatocyte | 36 RPM |
| | medium | Medium supplemented with ROCK inhibitor | |
| 2 | Xeno-free aggregation | LIFENET HEALTH ® Human Hepatocyte | 36 RPM |
| | medium | Medium supplemented with ROCK inhibitor, | |
| | | Pluronic F68, and DNase I | |
| 3 | Xeno-free aggregation | LIFENET HEALTH ® Human Hepatocyte | 80 RPM |
| | medium | Medium supplemented with ROCK inhibitor, | |
| | | Pluronic F68, and DNase I | |
| 4 | LIFENET HEALTH ® | LIFENET HEALTH ® Human Hepatocyte | 36 RPM |
| | Human Hepatocyte | Medium supplemented with ROCK inhibitor | |
| | Medium | | |
| 5 | LIFENET HEALTH ® | LIFENET HEALTH ® Human Hepatocyte | 36 RPM |
| | Human Hepatocyte | Medium supplemented with ROCK inhibitor, | |
| | Medium | Pluronic F68, and DNase I | |

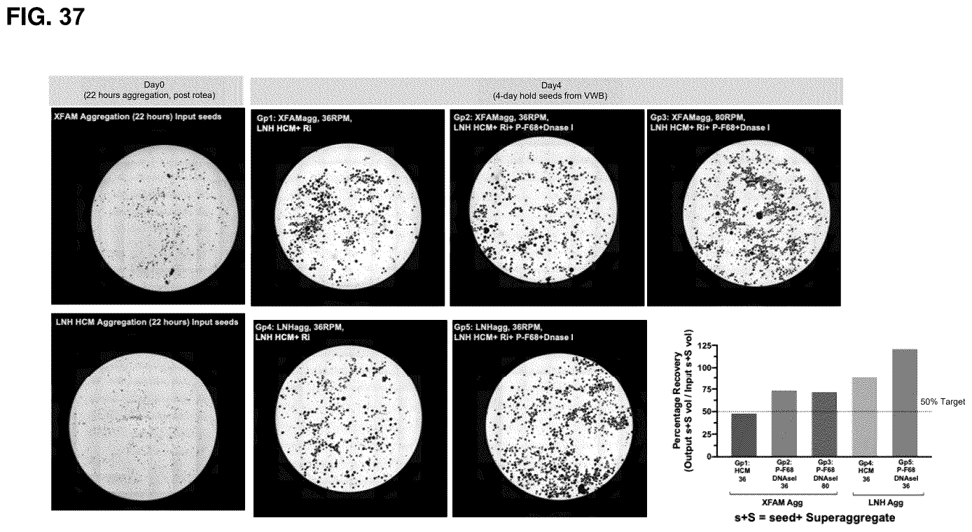

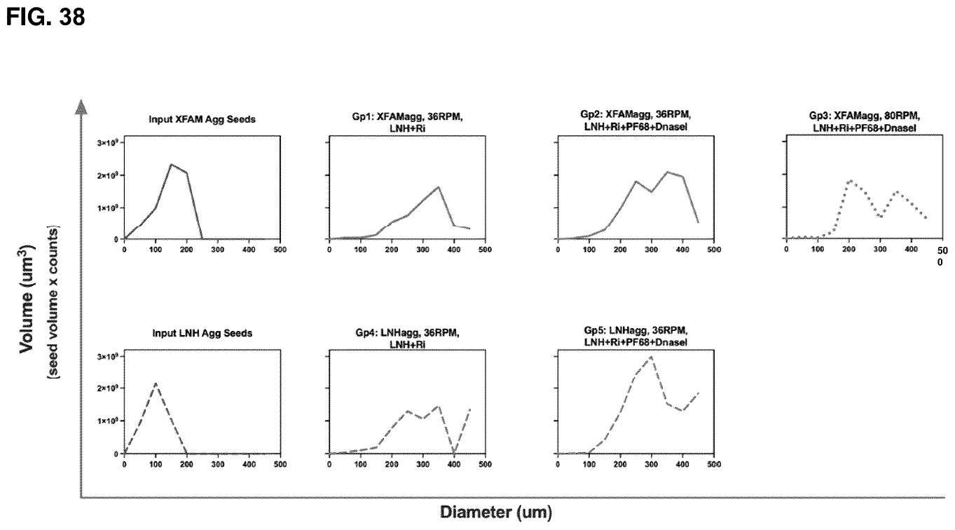

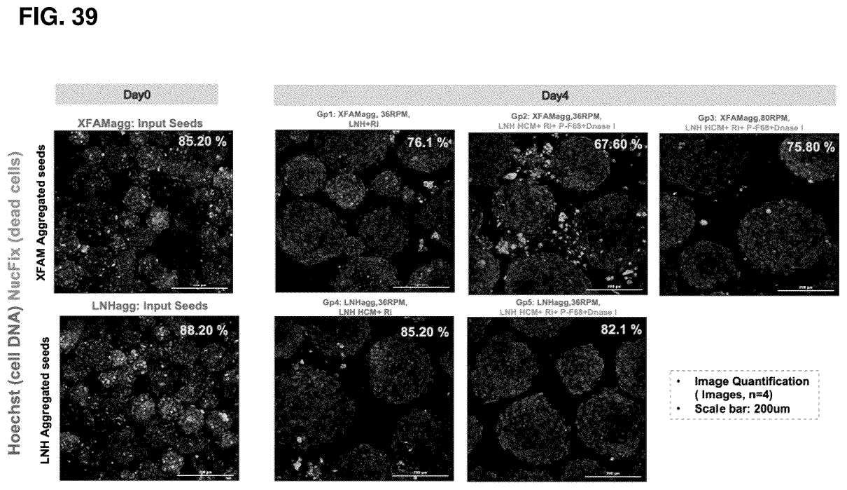

The initial aggregates at day 0 and the seeds that were recovered after four days of culturing under each condition are shown in FIG. 37. Additionally, the size distribution profiles of each condition are similar with Group 3 aggregates having a bimodal distribution at around 200 μm and 350 μm, as shown in FIG. 38. The initial input aggregates and recovered seeds at day 4 were assessed for viability via nuclei staining with NUCFIX™ or Hoeschst stain, in which most dead cells were present in the media rather than in the cell seeds themselves (FIG. 39).

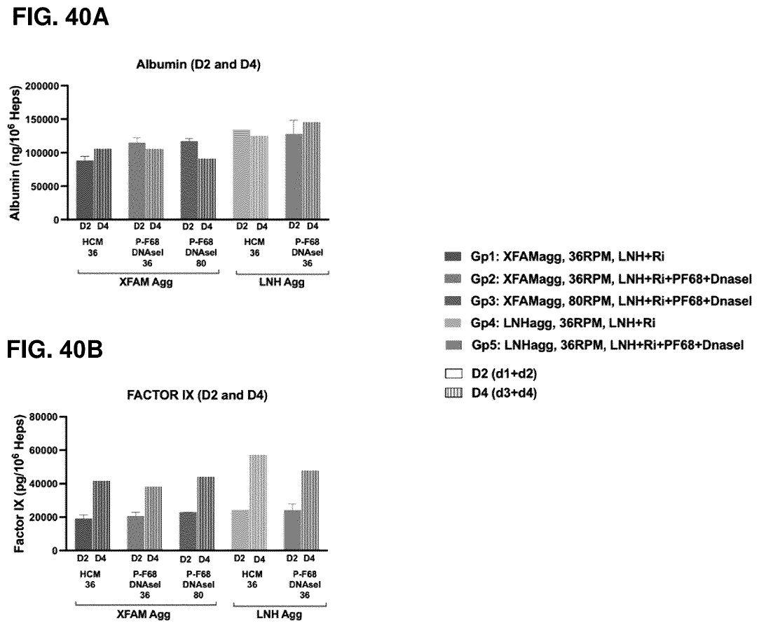

Hepatocyte identity was also assessed in the cell seeds. Secreted levels of albumin were similar across all conditions at both day 2 and day 4 of culturing (FIG. 40A). Additionally, secreted levels of factor IX were similar across all experimental groups, in which detectable levels of factor IX nearly doubled at day 4 compared to day 2 (FIG. 40B).

### Example 11. Expanded Hepatocyte Seeds

This example describes the assembly of hepatocyte seeds using hepatocytes that were expanded in Expand 3.0 or Expand 4.0 culture medium.

Cryopreserved populations of normal human dermal fibroblasts (NHDF) and expanded human hepatocytes that were expanded in either Expand 3.0 or Expand 4.0 media were thawed and combined at a ratio of 2:1 NHDF to expanded human hepatocytes. Seeds (20,000) were aggregated in LIFENET HEALTH® Human Hepatocyte Medium in VWBs. After 22 hours, the aggregates were washed in a Rotea centrifugation system (day 0) and were then cultured for four days in LIFENET HEALTH® Human Hepatocyte Medium supplemented with ROCK inhibitor at a rotor speed of 36 RPM.

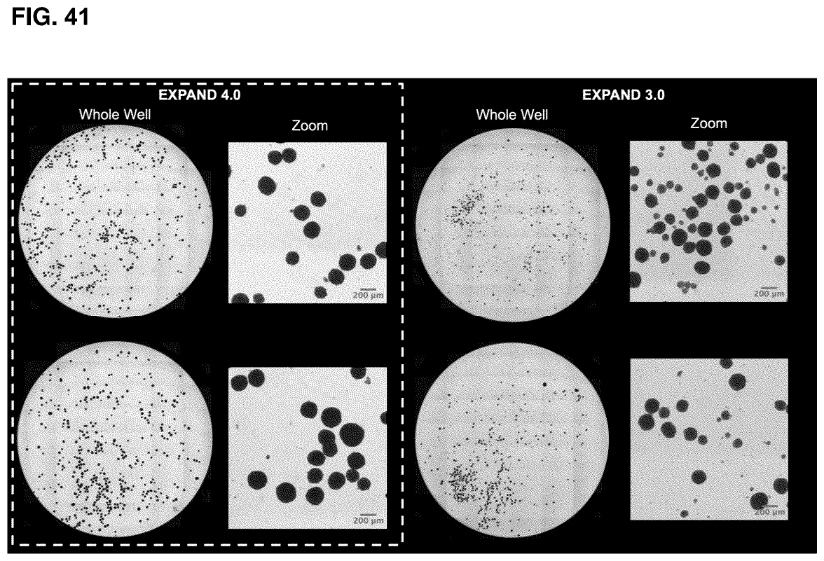

Seeds that were assembled with hepatocytes that were expanded in Expand 4.0 expansion medium across lots appeared consistently larger with less variation in seed size as compared to seeds assembled with hepatocytes that were expanded in Expand 3.0 Expansion medium (FIG. 41). Further, seeds produced with Expand 3.0-expanded hepatocytes, in addition to being smaller and having greater variation in size, had more cell debris compared to seeds produced with hepatocytes expanded in the Expand 4.0 expansion medium.

### Example 12. Hepatocyte Maturation In Vivo

This example describes the ability of expanded human hepatocytes in engrafting and maturing in a host when administered in vivo.

Seed aggregates (11 μL) were transplanted via intrasplenic pulp injection into NOD.Cg-Prkdescid Il2rg tm1 Wjl/SzJ (NSG) mice. All animals were provided with pre-surgical analgesics of: (i) subcutaneous administration of 0.6 mg/kg buprenorphine-SR, (ii) intraperitoneal administration of 10 mg/kg dexamethasone solution (DEXIUM®), and (iii) subcutaneous administration of 2 mg/kg ropivacaine at the incision site, and (iv) isoflurane inhalant for the surgical procedure.

Immediately prior to intrasplenic pulp injection, animals were administered 6 mg/kg enoxaparin low molecular weight heparin (Selleck Chem, S5415) via an intravenous injection in the lateral tail vein. Seeds at a dose of 11 μL were resuspended in William's E base media with 0.5% HSA to a total volume of 50 μL and drawn into a 100 μL gas-tight syringe fitted with a custom 25-gauge beveled needle. The needle and syringe were attached to a hand-held micro-infusion pump (World Precision Instruments), and the flow rate was set to 1.4 μL/sec. Additionally, animals received chronic dosing of Dexamethasone at 10 mg/kg by intraperitoneal administration for 4 consecutive days starting at post-operative day 1 and then every 2 days for the remainder of the study. Explanted spleen tissues were homogenized using Dounce homogenizer in Tissue Extraction Reagent I (Invitrogen) containing 1× Halt Protease and Phosphatase inhibitor Cocktail and equal amounts of protein (40-60 μg) were resolved by SDS-PAGE on a 12% or 4-12% acrylamide gel for western blot analysis. Equal protein loading was confirmed by Ponceau staining method.

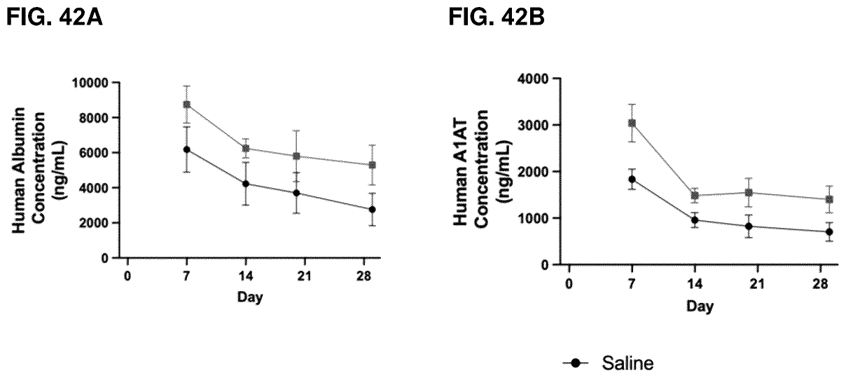

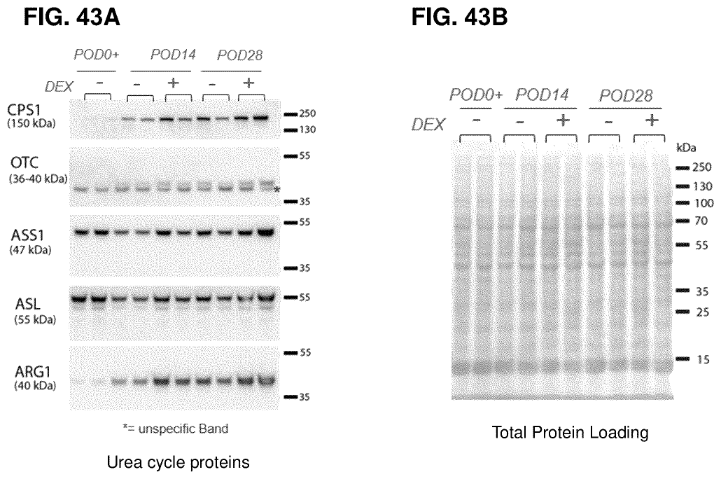

Following administration of the expanded hepatocytes, animals that were administered dexamethasone showed improved plasma concentrations of human albumin and human alpha-1 anti-trypsin (FIGS. 42A and 42B). Additionally, by post-operative day 14, homogenized liver tissue showed evidence of maturation by expression of urea cycle enzymes such as CPS1, ARG1, and OTC, with animals that received chronic dexamethasone treatment having increased expression of these enzymes compared to untreated animals (FIGS. 43A and 43B).

These data demonstrate that the hepatocytes expanded in the Expand 4.0 expansion medium are capable of maturing and functioning in vivo and that suppression of a host's immune system (e.g., via dexamethasone administration) improves hepatocyte engraftment.

### Example 13. Expanded Hepatocyte Engraftment of Various Extrahepatic Sites

This example describes successful engraftment and repopulation of hepatocytes in vivo following administration to various extrahepatic sites in a host.

Seeds were transplanted into NSG mice by either intrasplenic pulp injection, kidney capsule injection, or intraperitoneal implantation of grafts onto the fat pad. Pre-operative surgical procedures and intrasplenic pulp injection were performed as described in Example 8. Seeds transplanted into the kidney capsule were administered similarly as intrasplenic pulp administration described in Example 8, in which seeds were dispensed under the capsule toward the superior pole of the kidney. For seeds transplanted through grafts (e.g., on the fat pad), 11 μL seeds were encapsulated in 10 mm fibrin grafts and transported in William's E base medium with 0.5% HSA at 4° C. A 1.5 cm incision was made through the lower abdomen to expose the fat pad (parametrial fat pad in female animals and perigonadal fat pad in male animals).

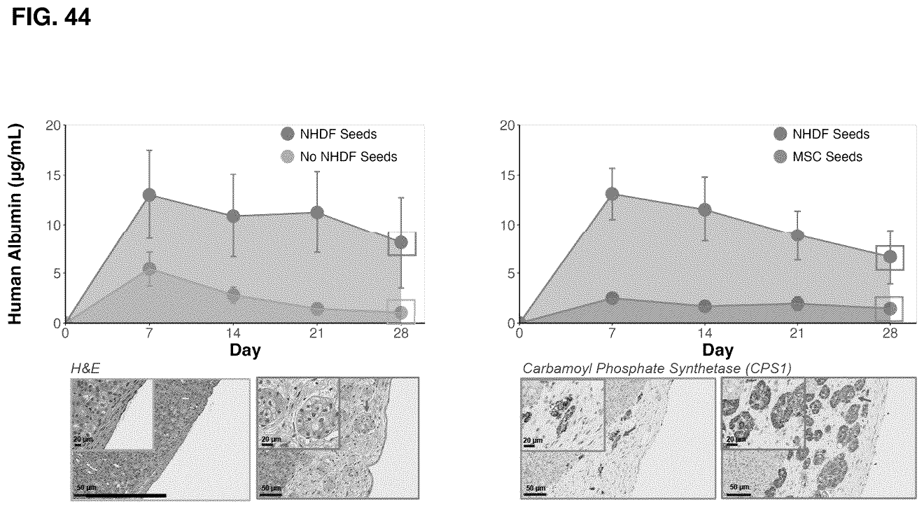

Engraftment potential of seeds in the kidney capsule of the NSG mouse model were evaluated by comparing NHDF-seeded constructs to fibroblast-free seeds and seeds aggregated with bone marrow-derived mesenchymal stromal cells (BM-MSCs). NHDF-containing seeds demonstrated significantly better engraftment compared to fibroblast-free and BM-MSC seeds, as demonstrated by elevated plasma human albumin levels and the presence of hepatocytes on explant, which was confirmed by hematoxylin and eosin (H&E) staining and increased expression of carbamoyl phosphate synthetase 1 (CPS1) (FIG. 44).

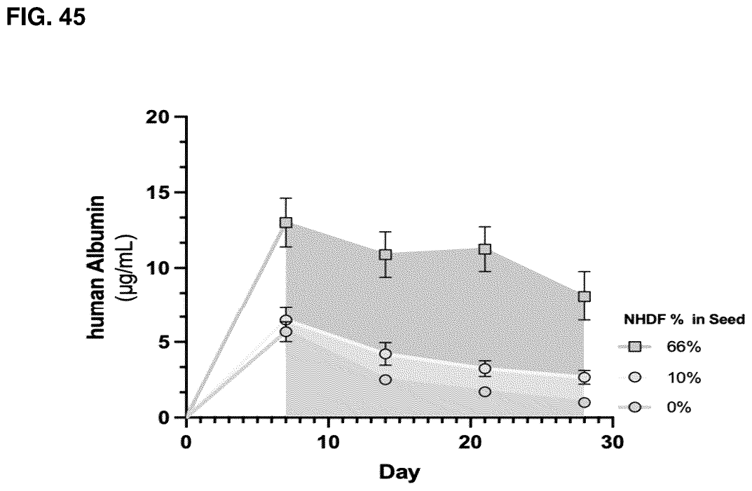

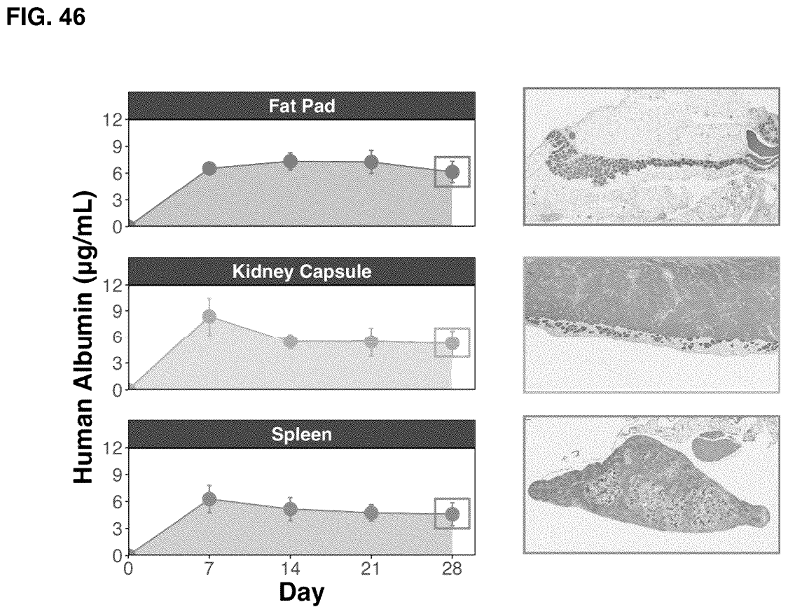

A 1:2 ratio of hepatocytes to fibroblasts achieved optimal engraftment compared to 1:10 or 1:0 ratios (FIG. 45). This ratio enabled successful engraftment across extrahepatic sites, including the spleen, fat pad, and kidney capsule, while maintaining stable human albumin levels and human OTC expression (FIG. 46).

### Example 14. Expanded Hepatocyte in an In Vivo Liver Injury Model

This example describes successful engraftment and repopulation of hepatocytes following administration of expanded human hepatocytes in a mouse model of hereditary tyrosinemia and improved survival following administration of the expanded human hepatocytes.

Fah^{-/-}/Rag2^{-/-}/Il2rg^{-/-} (FRG) mice were maintained on irradiated mouse chow with low tyrosine content and drinking water that contained 2-(2-nitro-4-trifluoromethylbenzoyl)-1,3-cyclohexanedione (NTBC) at a concentration of 16 mg/L until cell transplantation. NTBC was withdrawn from the drinking water of FRG animals the day before cell transplantation to initiate an NTBC on-and-off cycling scheme. An adenoviral vector expressing human urokinase (UPA, CURX™ uPA Liver Tx Enhancer, Yecuris Corporation) was injected at a dose of 5×10¹⁰ plaque forming units (pfu)/kg via tail vein intravenous injection the day before cell transplantation. A total of 0.65×10⁶ live PHHs, expanded human hepatocytes that were expanded in Expand 4.0 expansion medium (EHHs), matured and expanded human hepatocytes (EHHs+), iPSC-derived hepatocytes, or control cells were resuspended in 100 μL of XFAM media. Cells were transplanted via transabdominal intrasplenic injection using standard insulin syringes.

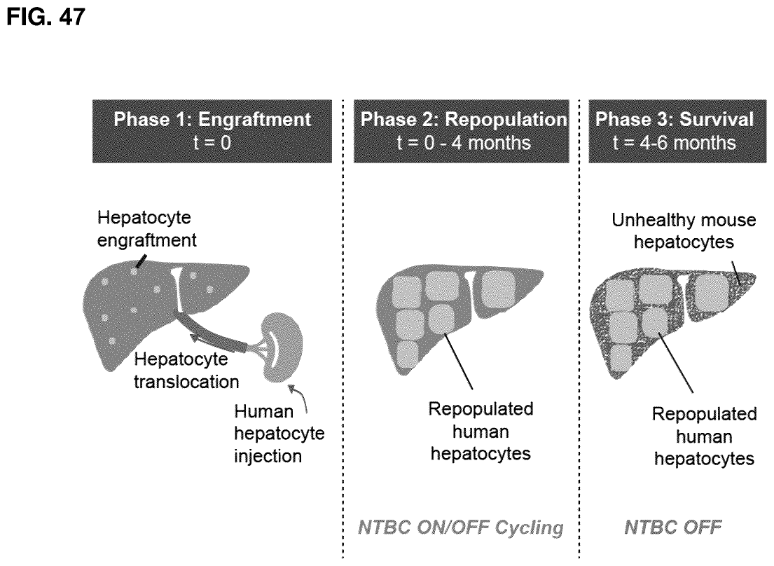

Over the course of 123 days, FRG animals were subjected to several NTBC on-and-off cycles of 7- to 9-day-off/3-day-on and 11- to 18-day-off/3-day-on schedules. NTBC concentration was maintained at 8 mg/L during the on days. After 123 days, NTBC was withdrawn from the drinking water until day 163. During this extended NTBC withdrawal period, the body weights of the animals were closely monitored, and cumulative survival was measured in each group. Endpoint criteria were defined as animals reaching a body weight reduction greater than 20%, a body condition score below 2, or death. The engraftment scheme for this hereditary tyrosinemia model is shown in (FIG. 47).

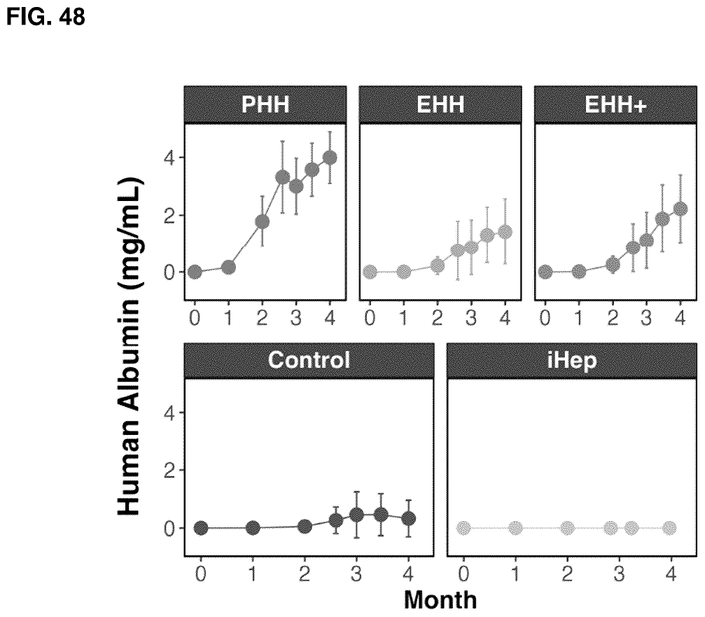

After injection into the spleen, the cells migrated from the spleen to the liver and subsequently repopulated the liver during NTBC on/off cycles. Expression levels of human albumin were measured in the blood of the animals (FIG. 48). By four months post-transplant, EHH and EHH+ cells demonstrated robust functionality, producing an average of 1.4 mg/mL and 2.2 mg/ml of human albumin, respectively. In comparison, PHHs produced 4 mg/mL, while iPSC-derived hepatocytes did not produce human albumin, and control cells produced an average of 0.3 mg/mL, respectively. In this experiment, the control cells consisted of EHHs cultured in a commercially available media formulation that led to poor in vivo engraftment of the cells.

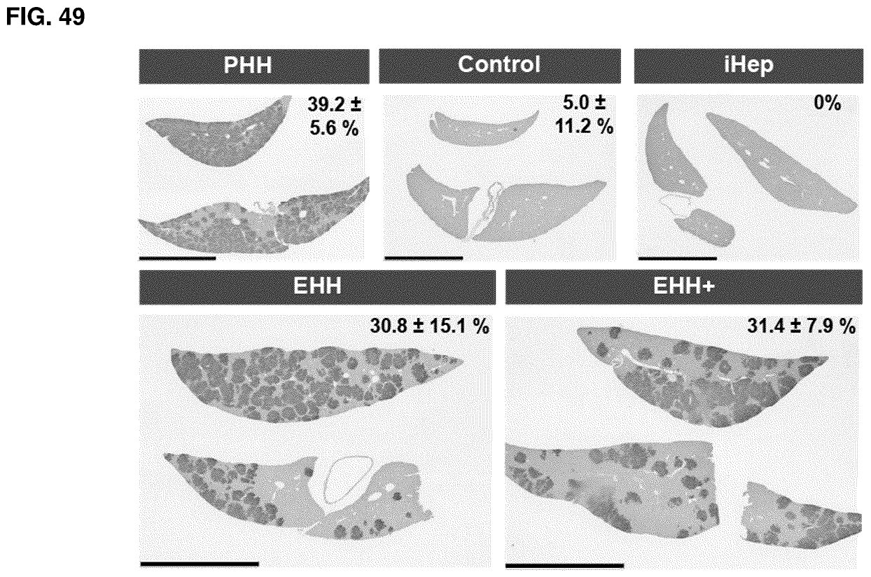

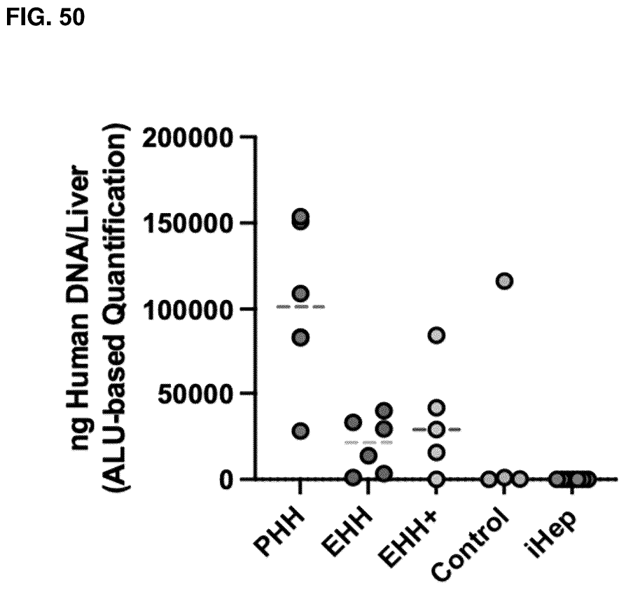

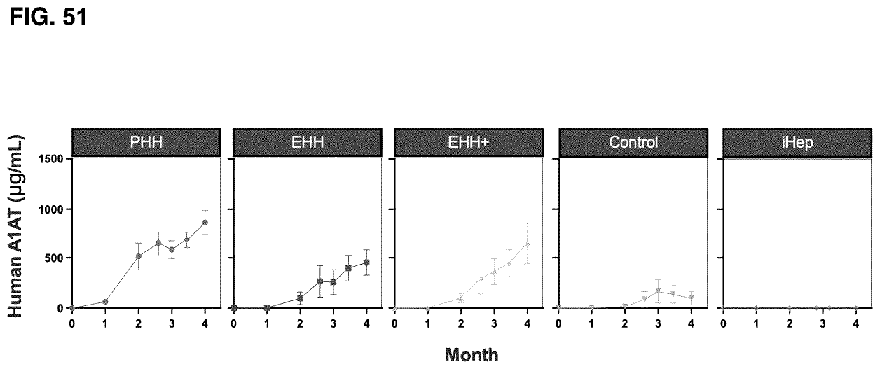

Histological staining for the human fumarylacetoacetate hydrolase (FAH) enzyme (FIG. 49) and quantification of human Alu sequences (FIG. 50) in mouse liver tissue confirmed repopulation. These results aligned with human albumin and alpha-1 antitrypsin measurements (FIG. 51).

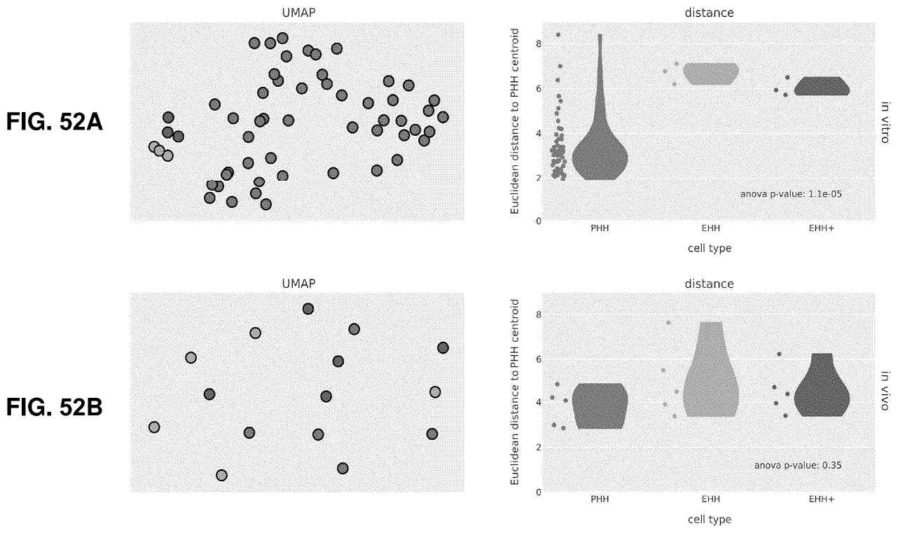

Additionally, bulk RNA-sequencing of the hepatocytes were performed. Gene expression from bulk RNA-sequencing was measured by incubating cells, seeds, or mouse tissue in Trizol and isolating mRNA using the PolyA capture method. The mRNA was run on a gel, and its quality was assessed with the RIN method, where a RIN>6 was accepted. Samples were then sequenced on an Illumina HiSeq 4000 with 2×150 bp paired-end sequencing. For in vitro samples, sequences from the raw FASTQ files were aligned to GRCh38 using RSubread with default parameters. For in vivo samples, human and mouse transcripts were disambiguated using XenofilteR with default parameters. The raw counts were normalized using gene exon length and sample sequencing depth to produce transcripts per million (TPM). These gene expression analyses showed that, initially, EHH and EHH+ cells exhibited distinct gene expression profiles compared to PHHs, as demonstrated by UMAP clustering and higher Euclidean distances (FIG. 52A). However, after four months in vivo in FRG mice, these differences diminished significantly (FIG. 52B). The cells became nearly indistinguishable, suggesting that the in vivo microenvironment plays a critical role in driving the maturation of EHHs and EHHs+ to closely resemble PHHs.

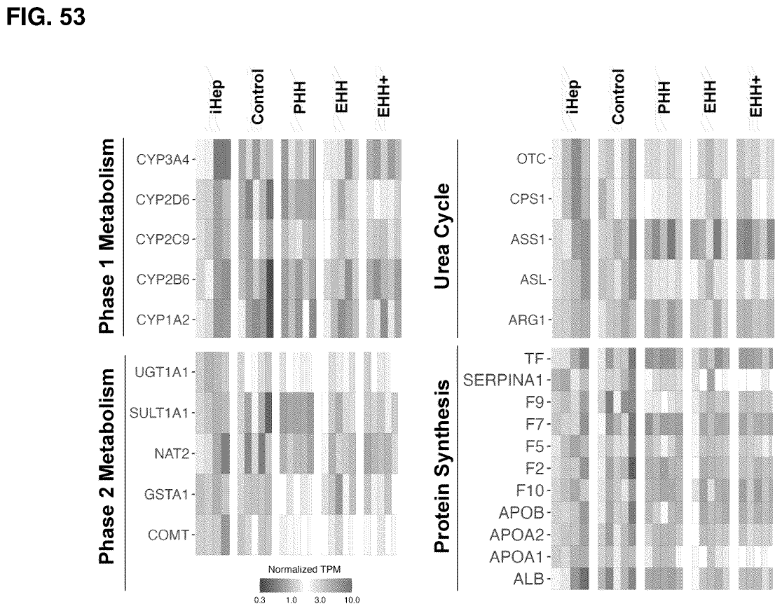

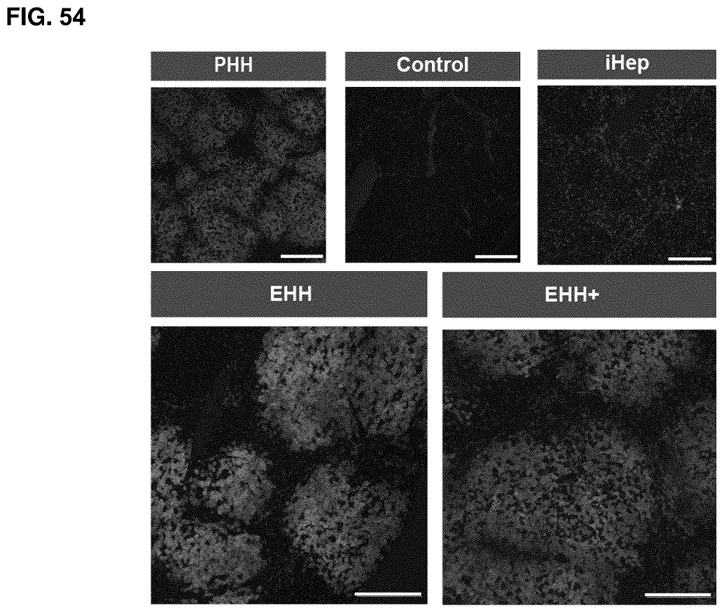

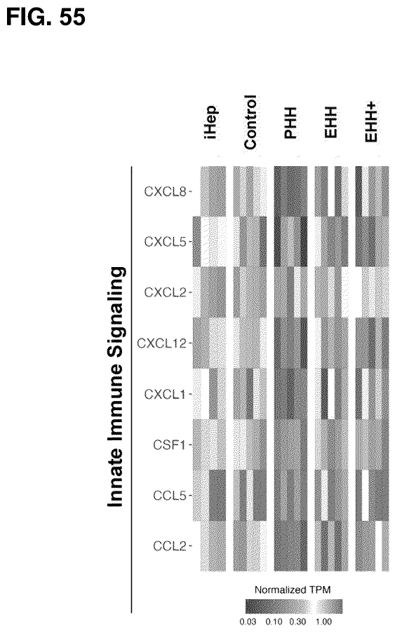

Moreover, gene expression analyses demonstrated that there was significant upregulation of genes associated with phase 1 and phase 2 metabolism, protein synthesis, and the urea cycle in engrafted cells in vivo (i.e., PHHs, EHHs, and EHHs+) compared to non-engrafted cells (i.e., iPSC-derived hepatocytes and the control EHHs cultured in commercially available medium) (FIG. 53). Staining of FRG mouse livers confirmed upregulation of ARG1 protein (FIG. 54). Additionally, cells exhibiting higher expression of chemokines and cytokines linked to innate immune signaling showed reduced engraftment efficiency. Notably, EHHs, EHHs+, and PHHs displayed lower expression levels of these immune-related molecules, correlating with their improved engraftment outcomes (FIG. 55).

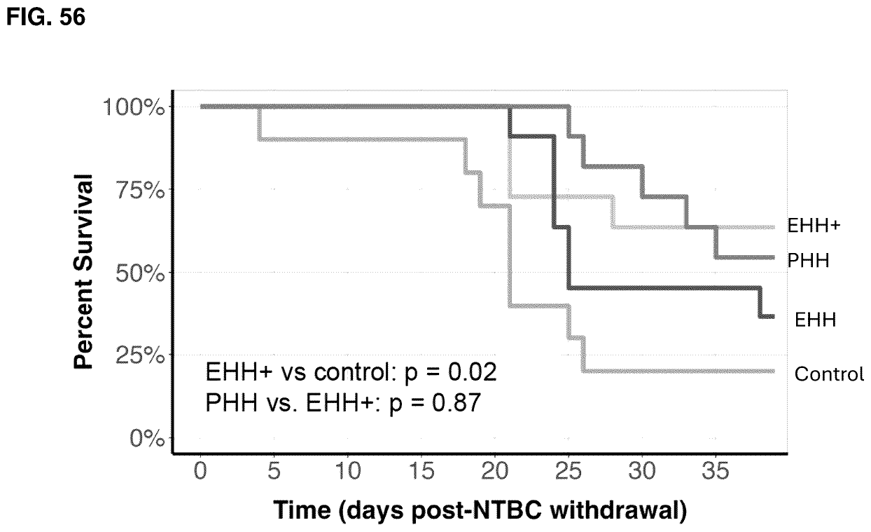

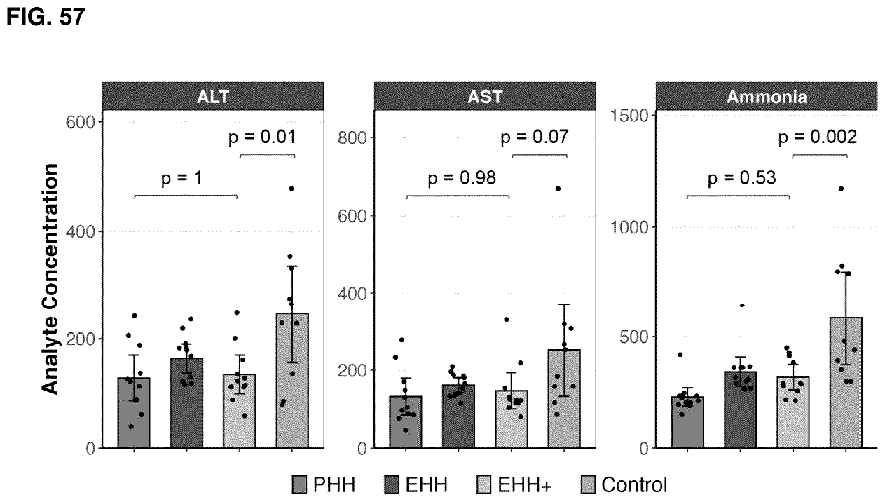

Finally, to assess therapeutic potential, the FRG mice were subjected to a survival challenge after four months via complete withdrawal of NTBC. During the NTBC OFF phase, EHHs+ demonstrated significantly improved survival compared to control cells (p=0.02), matching levels observed with PHHs (p=0.87) (FIG. 56). Liver injury markers (ALT, AST) and ammonia plasma levels were lowest in the EHH+ and PHH groups (FIG. 57), further demonstrating the potential safety and efficacy of these cells for therapeutic interventions in human subjects.

### Example 15. Expanded Hepatocytes Engraftment Following Intraarterial Administration

This example describes successful engraftment of expanded hepatocytes in the spleen of a larger animal model via intraarterial administration.

Göttingen minipigs were treated with an immunosuppression regimen consisting of methylprednisolone (administered intravenously at a single dose of 500 mg on Day 0), tacrolimus (daily oral administration of 0.4 mg/kg starting on Day-5), and abatacept-CTLA4-Ig (ORENCIA® administered intravenously at a single dose of 15 mg/kg IV on Day 0). On the day of seed transplantation, fasted animals were pre-anesthetized and administered prophylactic antibiotics and analgesics. Animals were intubated and maintained on isoflurane for effect during surgery.

Prior to surgery, seeds were prepared by loading 1×10⁷ seeds/kg suspended in 15 mL DMEM+0.5% human serum albumin+1% penicillin and streptomycin into a cryostorage bag. Seed infusion into the splenic artery of the animal was performed at a flow rate of 3 mL/min using a controlled infusion pump (Jorgensen Lab, 1060Q). Wash media (DMEM+0.5% human serum albumin, up to 20 mL) was used to resuspend the seeds in the bag during infusion and to wash the bag and IV line after the seed infusion was completed. Sodium heparin was administered intravenously during infusion at 100-300 units/kg to prevent clot formation, followed by 100-200 units/kg every 45-60 minutes.

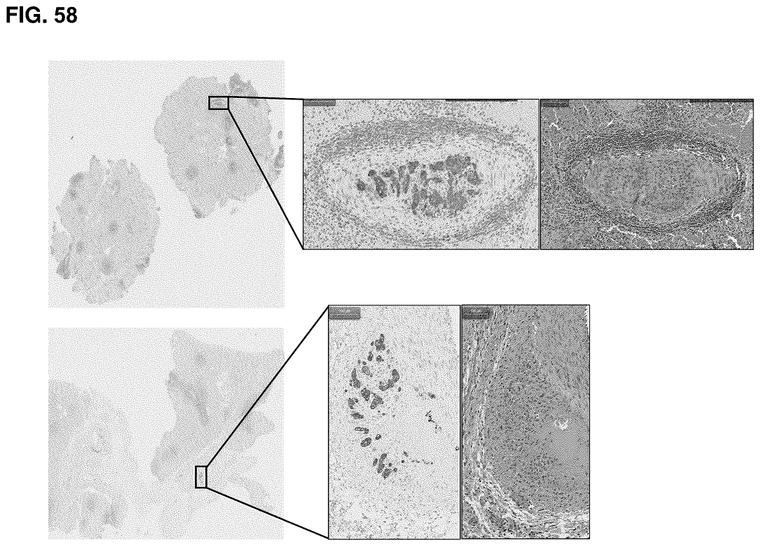

The procedure was well tolerated, all animals fully recovered, and no adverse events were reported. Biopsy analysis using immunohistochemistry for cytokeratin 18 (CK18) (FIG. 58) and quantification of human Alu sequences via real-time PCR following DNA extraction of homogenized tissues (Tables 11 and 12) revealed that the seeds largely remained localized to the spleen, unlike single-cell hepatocyte transplants, which typically migrate to the liver and can have the potential for a wider off-target distribution to other organ systems resulting in safety concerns.

TABLE 11
Biodistribution of seeds in target organs

| | Copies/μg Genomic DNA | |
|---|---|---|
| Biopsy tissue | Animal 1 | Animal 2 |

Spleen

| | | |
|---|---|---|
| Spleen_1 (dorsal/proximal extremity) | 486.68 | <LOQ |
| Spleen_2 (body, lateral to midline/hilus) | 708.58 | <LOQ |
| Spleen_3 (body, lateral to midline/hilus) | 562.79 | <LOQ |
| Spleen_4 (body, lateral to midline/ hilus) | 134.80 | <LOQ |
| Spleen_5 (ventral/distal extremity) | <LOQ | 7352.88 |

Liver

| | | |
|---|---|---|
| Left lateral hepatic lobe, distal | 120.09 | 2.43 |
| Left lateral hepatic lobe, proximal | 5.25 | 7.69 |
| Left median hepatic lobe, distal | 24.88 | 13.35 |
| Left median hepatic lobe, proximal | 104.16 | <LOQ |
| Right median hepatic lobe, distal | 60.52 | 2.03 |
| Right median hepatic lobe, proximal | <LOQ | <LOQ |
| Right lateral hepatic lobe, distal | <LOQ | <LOQ |
| Right lateral hepatic lobe, proximal | <LOQ | <LOQ |

LOQ: Limit of quantification

TABLE 12
Biodistribution of seeds in off-target organs

Copies/μg Genomic DNA

| | | |
|---|---|---|
| Biopsy tissue | Animal 1 | Animal 2 |

Kidney

| | | |
|---|---|---|
| Renal capsule | <LOQ | <LOQ |
| Renal parenchyma | <LOQ | <LOQ |

Lung

| | | |
|---|---|---|
| Left lower pulmonary lobe | <LOQ | <LOQ |
| Left upper pulmonary lobe | <LOQ | <LOQ |
| Right upper pulmonary lobe | <LOQ | <LOQ |
| Right middle pulmonary lobe | <LOQ | <LOQ |
| Right lower pulmonary lobe | <LOQ | <LOQ |

These data emphasize that seeds comprising expanded hepatocytes can localize and engraft in desired extrahepatic sites (e.g., sites in the spleen) following intraarterial delivery, which can translate to therapeutic and safety benefits for treatment of human subjects.

### Specific Embodiments

Several non-limiting, exemplary embodiments of the disclosure are enumerated below. The below embodiments should not be construed to limit the scope of the invention, rather, the below are presented as some examples of the utility of the invention.

1. A method of culturing hepatocytes, the method comprising culturing one or more hepatocytes in contact with an ECM in the presence of an expansion medium comprising a basal cell culture medium for human cells to which is added: an FGF, an EGF, an HGF, an R-spondin, and a TGF-beta inhibitor, wherein one or more of FGF7, Wnt3a, and TGF-alpha are not added to the culture medium.

2. A method of culturing hepatocytes, the method comprising culturing one or more hepatocytes in contact with an ECM in the presence of an expansion medium comprising a basal cell culture medium for human cells to which is added: an FGF, an HGF, TGF-alpha, and a TGF-beta inhibitor, wherein one or more of FGF7, an EGF, Wnt3a, and an R-spondin are not added to the culture medium.

3. The method of embodiment 1 or 2, wherein the FGF added to the basal cell culture medium is not FGF7.

4. The method of embodiment 1, wherein two or more of FGF7, Wnt3a, and TGF-alpha are not added to the basal cell culture medium.

5. The method of embodiment 3, wherein FGF7, Wnt3a, and TGF-alpha are not added to the basal cell culture medium.

6. The method of embodiment 2, wherein two or more of FGF7, an EGF, Wnt3a, and an R-spondin are not added to the culture medium.

7. The method of embodiment 2 or 6, wherein three or more of FGF7, an EGF, Wnt3a, and an R-spondin are not added to the culture medium.

8. The method of any one of embodiments 2, 6, and 7, wherein FGF, an EGF, Wnt3a, and an R-spondin are not added to the culture medium.

9. The method of any one of embodiments 1 and 3-5, wherein the R-spondin comprises R-spondin 1, R-spondin 2, R-spondin 3, R-spondin 4, or a combination thereof.

10. The method of embodiment 9, wherein the R-spondin comprises R-spondin 1.

11. The method of any one of embodiments 1-10, further comprising adding an antioxidant to the basal cell culture medium.

12. The method of embodiment 11, wherein the antioxidant comprises N-acetyl cysteine, nicotinamide, or a combination thereof.

13. The method of any one of embodiments 1-12, further comprising adding a cell survival agent to the basal cell culture medium.

14. The method of embodiment 13, wherein the cell survival agent is a supplement for a serum-free cell medium.

15. The method of embodiment 14, wherein the supplement comprises a B27 supplement, an N2 supplement, or a combination thereof.

16. The method of embodiment 14 or 15, wherein the supplement does not comprise vitamin A.

17. The method of any one of embodiments 1-16, wherein the FGF comprises FGF2, FGF3, FGF10, FGF22, or a combination thereof.

18. The method of embodiment 17, wherein the FGF comprises FGF10.

19. The method of any one of embodiments 1-18, wherein the TGF-beta inhibitor comprises an inhibitor of ALK5.

20. The method of embodiment any one of embodiments 1-19, wherein the TGF-beta inhibitor comprises A83-01.

21. The method of any one of embodiments 1-20, further comprising adding an amino acid supplement to the basal cell culture medium.

22. The method of embodiment 21, wherein the amino acid supplement comprises an NEAA supplement, an L-glutamine substitute, or a combination thereof.

23. The method of any one of embodiments 1-22, further comprising adding a serum replacement component to the basal cell culture medium.

24. The method of embodiment 23, wherein the serum replacement component comprises KOSR.

25. The method of any one of embodiments 1-24, further comprising adding a buffering agent to the basal cell culture medium.

26. The method of embodiment 25, wherein the buffering agent comprises HEPES.

27. The method of any one of embodiments 1-26, wherein the ECM comprises a laminin, a collagen, a vitronectin, a fibronectin, or a combination thereof.

28. The method of any one of embodiments 1-27, wherein the ECM comprises a xeno-free substrate.

29. The method of embodiment 27 or 28, wherein the laminin is laminin-111, laminin-211, laminin-221, laminin-332, laminin-411, laminin-421, laminin-511, or laminin-521.

30. The method of embodiment 29, wherein the laminin is laminin-521.

31. The method of any one of embodiments 1-30, wherein the ECM is applied to a surface.

32. The method of embodiment 31, wherein the ECM is applied to the surface at a density of 0.1 μg/cm² to 0.7 μg/cm².

33. The method of embodiment 32, wherein the ECM is applied to the surface at a density of 0.125 μg/cm².

34. The method of any one of embodiments 1-33, wherein the hepatocytes are PHHs or iPSC-derived hepatocytes.

35. The method of embodiment 34, wherein the PHHs were previously cryopreserved.

36. The method of embodiment 35, wherein the PHHs have not been previously cryopreserved.

37. The method of any one of embodiments 1-36, wherein the method comprises expanding plated hepatocytes (step P0) and a first passage of expanded hepatocytes (step P1).

38. The method of embodiment 37, wherein step P0 has a duration of 5 to 25 days.

39. The method of embodiment 37, wherein step P0 has a duration of 10 to 14 days.

40. The method of any one of embodiments 37-39, wherein the expansion medium is replaced every 24 to 48 hours or 24 to 72 hours during step P0.

41. The method of any one of embodiments 37-40, wherein at step P0, the hepatocytes are seeded at a cell density of 300 viable cells/cm² to 13,000 viable cells/cm².

42. The method of any one of embodiments 37-41, wherein step P1 has a duration of 7 to 16 days.

43. The method of embodiment 42, wherein step P1 has a duration of 10 to 14 days.

44. The method of any one of embodiments 37-43, wherein the expansion medium is replaced every 24 to 48 hours or 24 to 72 hours during step P1.

45. The method of any one of embodiments 37-44, wherein at step P1, the hepatocytes are seeded at a cell density of 300 viable cells/cm² to 13,000 viable cells/cm².

46. The method of any one of embodiments 37-45, wherein step P0 and step P1 have a total duration of 10 to 50 days.

47. The method of any one of embodiments 37-46, wherein the population of hepatocytes expand by at least 10-fold or at least 100-fold during step P0 and/or by at least 500-fold or at least 10,000 fold cumulatively in step P0 and step P1.

48. The method of any one of embodiments 1-47, wherein at least 70% of the hepatocytes are viable following the culturing.

49. The method of any one of embodiments 1-48, further comprising culturing the hepatocytes in a maturation medium comprising a basal cell culture medium for human cells to which one or more maturation supplements are added.

50. The method of embodiment 49, wherein the hepatocytes are cultured in the maturation medium for a duration of 3 days to 7 days.

51. The method of embodiment 50, wherein the hepatocytes are cultured in the maturation medium for a duration of 4 days.

52. The method of any one of embodiments 49-51, wherein culturing in the maturation medium begins immediately after hepatocyte expansion.

53. The method of any one of embodiments 49-51, wherein the expanded hepatocytes have been cryopreserved prior to culturing in the maturation medium.

54. The method of any one of embodiments 49-53, wherein the basal cell medium is selected from the group consisting of Takara Cellartis POWER™ Primary HEP medium, Lonza HCM™ cell culture medium, William's E medium, and LIFENET HEALTH® Human Hepatocyte Media.

55. The method of any one of embodiments 49-54, wherein the one or more maturation supplements comprise HEPES, an L-glutamine substitute, insulin transferrin selenium, a Notch inhibitor, an EGFR inhibitor, Oncostatin M, an antioxidant, a glucocorticoid, a PXR activator, a bile acid, a CAMP or CAMP analog, cholesterol, a thyroid hormone, a serum replacement component, or any combination thereof.

56. The method of embodiment 55, wherein the Notch inhibitor comprises Compound E, Gamma Secretase Inhibitor XX, or a combination thereof.

57. The method of embodiment 55 or 56, wherein the EGFR inhibitor comprises erlotinib HCl.

58. The method of any one of embodiments 55-57, wherein the antioxidant comprises vitamin C.

59. The method of any one of embodiments 55-58, wherein the glucocorticoid comprises dexamethasone, hydrocortisone, or a combination thereof.

60. The method of any one of embodiments 55-59, wherein the PXR activator is vitamin K2, lithocholic acid, or a combination thereof.

61. The method of any one of embodiments 55-60, wherein the bile acid is lithocholic acid, urso deoxycholic acid, or a combination thereof.

62. The method of any one of embodiments 55-61, wherein the CAMP analog is 8-bromo CAMP, forskolin, or a combination thereof.

63. The method of any one of embodiments 55-62, wherein the thyroid hormone is T3.

64. The method of any one of embodiments 55-63, further comprising adding an amino acid supplement to the basal cell culture medium.

65. The method of embodiment 64, wherein the amino acid supplement comprises an NEAA supplement, an EAA supplement, or a combination thereof.

66. The method of any one of embodiments 55-65, further comprising adding a peptide hormone to the basal cell culture medium.

67. The method of embodiment 66, wherein the peptide hormone comprises glucagon.

68. The method of any one of embodiments 55-67, further comprising adding a DNase to the basal cell culture medium.

69. The method of any one of embodiments 55-68, further comprising adding a ROCK inhibitor.

70. The method of any one of embodiment 55-69, further comprising adding a non-ionic detergent.

71. The method of embodiment 70, wherein the non-ionic detergent comprises Pluronic F68.

72. The method of any one of embodiments 49-67, wherein the hepatocytes are cultured in the maturation medium on a surface of a cell culture vessel.

73. The method of embodiment 72, wherein the surface of the cell culture vessel is coated with an ECM.

74. The method of any one of embodiments 49-71, wherein following hepatocyte expansion, the hepatocytes are admixed with a population of stromal cells to form a seed.

75. The method of embodiment 74, wherein the population of stromal cells comprises fibroblasts.

76. The method of embodiment 75, wherein the fibroblasts are normal human dermal fibroblasts. 77. The method of any one of embodiments 1-76, wherein the method comprises determining the expression profile of the hepatocytes following culturing.

78. The method of embodiment 77, wherein the expression profile of the hepatocytes comprises expression of one or more biomarkers selected from HNF4-alpha, CD81, ASGR1, NR112, A1AT, CYP3A4, CYP1A2, factor IX, and/or albumin by at least 70% of the hepatocytes.

79. The method of embodiment 78, wherein the expression profile of the hepatocytes comprises expression of one or more biomarkers selected from HNF4-alpha, CD81, ASGR1, NR112, A1AT, CYP3A4, CYP1A2, factor IX, and/or albumin by at least 80% of the hepatocytes.

80. The method of any one of embodiments 77-79, wherein the expression profile of the hepatocytes further comprises expression of one or more urea cycle enzymes by at least 70% of the hepatocytes.

81. The method of embodiment 80, wherein the expression profile of the hepatocytes further comprises expression of one or more urea cycle enzymes by at least 80% of the hepatocytes.

82. The method of embodiment 80 or 81, wherein the one or more urea cycle enzymes comprise ARG1, ASL, ASS1, CPS1, NAGS, OTC, or a combination thereof.

83. The method of any one of embodiments 77-82, wherein the expression profile of the hepatocytes comprises expression of one or more progenitor markers and/or cholangiocyte markers by 10% or fewer of the hepatocytes.

84. The method of embodiment 83, wherein the one or more progenitor markers comprise AFP, LGR5, CYP3A7, EpCAM, TBX3, or a combination thereof.

85. The method of embodiment 83 or 84, wherein the one or more cholangiocyte markers comprise AQP1, KRT19, KRT7, TFF1, TFF2, or a combination thereof.

86. A method of maturing hepatocytes, the method comprising culturing iPSC-derived hepatocytes in a maturation medium comprising a basal cell culture medium for human cells to which one or more maturation supplements are added.

87. The method of embodiment 86, wherein the hepatocytes are cultured in the maturation medium for a duration of 3 days to 7 days.

88. The method of embodiment 87, wherein the hepatocytes are cultured in the maturation medium for a duration of 4 days.

89. The method of any one of embodiments 86-88, wherein the basal cell medium is selected from the group consisting of Takara Cellartis POWER™ Primary HEP medium, Lonza HCM™ cell culture medium, William's E medium, and LIFENET HEALTH® Human Hepatocyte Media.

90. The method of any one of embodiments 86-89, wherein the one or more maturation supplements comprise HEPES, an L-glutamine substitute, insulin transferrin selenium, a Notch inhibitor, an EGFR inhibitor, Oncostatin M, an antioxidant, a glucocorticoid, a PXR activator, a bile acid, a cAMP or CAMP analog, cholesterol, a thyroid hormone, a serum replacement component, an amino acid supplement, a peptide hormone, or any combination thereof.

91. The method of embodiment 90, wherein the Notch inhibitor comprises Compound E, Gamma Secretase Inhibitor XX, or a combination thereof.

92. The method of embodiment 90 or 91, wherein the EGFR inhibitor comprises erlotinib HCl.

93. The method of any one of embodiments 90-92, wherein the antioxidant comprises vitamin C.

94. The method of any one of embodiments 90-93, wherein the glucocorticoid comprises dexamethasone, hydrocortisone, or a combination thereof.

95. The method of any one of embodiments 90-94, wherein the PXR activator is vitamin K2, lithocholic acid, or a combination thereof.

96. The method of any one of embodiments 90-95, wherein the bile acid is lithocholic acid, urso deoxycholic acid, or a combination thereof.

97. The method of any one of embodiments 90-96, wherein the CAMP analog is 8-bromo CAMP, forskolin, or a combination thereof.

98. The method of any one of embodiments 90-97, wherein the thyroid hormone is T3.

99. The method of embodiment 90-98, wherein the amino acid supplement comprises an NEAA supplement, an EAA supplement, or a combination thereof.

100. The method of embodiment 90-99, wherein the peptide hormone comprises glucagon.

101. The method of any one of embodiments 86-100, further comprising adding a DNase to the basal cell culture medium.

102. The method of any one of embodiments 86-101, further comprising adding a ROCK inhibitor.

103. The method of any one of embodiment 86-102, further comprising adding a non-ionic detergent.

104. The method of embodiment 103, wherein the non-ionic detergent comprises Pluronic F68.

105. The method of any one of embodiments 86-100, wherein the hepatocytes are cultured in the maturation medium on a surface of a cell culture vessel.

106. The method of embodiment 105, wherein the surface of the cell culture vessel is coated with an ECM.

107. The method of any one of embodiments 86-104, wherein following hepatocyte expansion, the hepatocytes are admixed with a population of stromal cells to form a seed.

108. The method of embodiment 107, wherein the population of stromal cells comprises fibroblasts.

109. The method of embodiment 108, wherein the fibroblasts are normal human dermal fibroblasts.

110. The method of any one of embodiments 86-109, wherein the method comprises determining the expression profile of the hepatocytes following culturing.

111. The method of embodiment 110, wherein the expression profile of the hepatocytes comprises expression of one or more biomarkers selected from HNF4-alpha, CD81, ASGR1, NR112, A1AT, CYP3A4, CYP1A2, factor IX, prothrombin, and/or albumin by at least 70% of the hepatocytes.

112. The method of embodiment 111, wherein the expression profile of the hepatocytes comprises expression of one or more biomarkers selected from HNF4-alpha, CD81, ASGR1, NR112, A1AT, CYP3A4, CYP1A2, factor IX, prothrombin, and/or albumin by at least 80% of the hepatocytes.

113. The method of any one of embodiments 86-112, wherein the expression profile of the hepatocytes further comprises expression of one or more urea cycle enzymes by at least 70% of the hepatocytes.

114. The method of embodiment 113, wherein the expression profile of the hepatocytes further comprises expression of one or more urea cycle enzymes by at least 80% of the hepatocytes.

115. The method of embodiment 113 or 114, wherein the one or more urea cycle enzymes comprise ARG1, ASL, ASS1, CPS1, NAGS, OTC, or a combination thereof.

116. The method of any one of embodiments 110-115, wherein the expression profile of the hepatocytes comprises expression of one or more progenitor markers and/or cholangiocyte markers by 10% or fewer of the hepatocytes.

117. The method of embodiment 116, wherein the one or more progenitor markers comprise AFP, LGR5, CYP3A7, EpCAM, TBX3, or a combination thereof. 118. The method of embodiment 116 or 117, wherein the one or more cholangiocyte markers comprise AQP1, KRT19, KRT7, TFF1, TFF2, or a combination thereof.

119. The method of any one of embodiments 86-118, wherein prior to maturing the hepatocytes, the hepatocytes are cultured according to a method of any one of embodiments 1-48.

120. The method of any one of embodiments 86-118, wherein prior to maturing the hepatocytes, the hepatocytes are cultured in contact with an ECM in the presence of an expansion medium comprising a basal cell culture medium for human cells to which is added: an FGF, an HGF, and a TGF-beta inhibitor.

121. The method of embodiment 120, further comprising adding an R-spondin to the basal cell culture medium.

122. The method of embodiment 121, wherein the R-spondin comprises R-spondin 1, R-spondin 2, R-spondin 3, R-spondin 4, or a combination thereof.

123. The method of embodiment 122, wherein the R-spondin comprises R-spondin 1.

124. The method of any one of embodiments 120-123, wherein the FGF comprises FGF2, FGF3, FGF7, FGF10, FGF22, or a combination thereof.

125. The method of any one of embodiments 116-119, further comprising adding an EGF and/or Wnt3a to the basal cell culture medium.

126. The method of any one of embodiments 120-125, further comprising adding TGF-alpha to the basal cell culture medium.

127. The method of any one of embodiments 120-126, further comprising adding an antioxidant to the basal cell culture medium.

128. The method of embodiment 127, wherein the antioxidant comprises N-acetyl cysteine, nicotinamide, or a combination thereof.

129. The method of any one of embodiments 120-128, further comprising adding a cell survival agent to the basal cell culture medium.

130. The method of embodiment 129, wherein the cell survival agent is a supplement for a serum-free cell medium.

131. The method of embodiment 130, wherein the supplement comprises a B27 supplement, an N2 supplement, or a combination thereof.

132. The method of embodiment 130 or 131, wherein the supplement does not comprise vitamin

A 133. The method of any one of embodiments 120-132, wherein the TGF-beta inhibitor is an ALK5 inhibitor.

134. The method of embodiment 133, wherein the ALK5 inhibitor is A83-01.

135. The method of any one of embodiments 120-134, further comprising adding an amino acid supplement to the basal cell culture medium.

136. The method of embodiment 135, wherein the amino acid supplement comprises an NEAA supplement, an L-glutamine substitute, or a combination thereof.

137. The method of any one of embodiments 120-136, further comprising adding a serum replacement component to the basal cell culture medium. 138. The method of embodiment 137, wherein the serum replacement component comprises KOSR.

139. The method of any one of embodiments 120-138, further comprising adding a buffering agent to the basal cell culture medium.

140. The method of embodiment 139, wherein the buffering agent comprises HEPES.

141. The method of any one of embodiments 120-140, wherein the ECM comprises a laminin, a collagen, a vitronectin, a fibronectin, or a combination thereof.

142. The method of any one of embodiments 120-141, wherein the ECM comprises a xeno-free substrate.

143. The method of embodiment 141 or 142, wherein the laminin is laminin-111, laminin-211, laminin-221, laminin-332, laminin-411, laminin-421, laminin-511, or laminin-521.

144. The method of embodiment 143, wherein the laminin is laminin-521.

145. The method of any one of embodiments 120-144, wherein the ECM is applied to a surface.

146. The method of embodiment 145, wherein the ECM is applied to the surface at a density of 0.1 g/cm² to 0.7 μg/cm².

147. The method of any one of embodiments 1-146, wherein the hepatocytes following culturing are administered to a liver of a subject.

148. A cell culture medium comprising a basal cell culture medium for human cells and an exogenous source of an FGF, an EGF, an HGF, an R-spondin, and a TGF-beta inhibitor, wherein the cell culture medium does not comprise an exogenous source of one or more of FGF7, Wnt3a, and TGF-alpha.

149. A cell culture medium comprising a basal cell culture medium for human cells and an exogenous source of an FGF, an HGF, a TGF-alpha, and a TGF-beta inhibitor, wherein the cell culture medium does not comprise an exogenous source of one or more of FGF7, an EGF, Wnt3a, and an R-spondin.

150. The cell culture medium of embodiment 148 or 149, wherein the FGF is not FGF7.

151. The cell culture medium of embodiment 148, wherein the cell culture medium does not comprise an exogenous source of two or more of FGF7, Wnt3a, and TGF-alpha.

152. The cell culture medium of embodiment 151, wherein the cell culture medium does not comprise an exogenous source of FGF7, Wnt3a, and TGF-alpha.

153. The cell culture medium of embodiment 149, wherein the cell culture medium does not comprise an exogenous source of two or more of FGF7, an EGF, Wnt3a, and an R-spondin.

154. The cell culture medium of embodiment 149 and 153, wherein the cell culture medium does not comprise an exogenous source of three or more of FGF7, an EGF, Wnt3a, and an R-spondin.

155. The cell culture medium of any one of embodiments 149, 153, and 154, wherein the cell culture medium does not comprise an exogenous source of FGF7, an EGF, Wnt3a, and an R-spondin.

156. The cell culture medium of any one of embodiments 148 and 150-152, wherein the R-spondin comprises R-spondin 1, R-spondin 2, R-spondin 3, R-spondin 4, or a combination thereof.

157. The cell culture medium of embodiment 156, wherein the R-spondin comprises R-spondin 1.

158. The cell culture medium of any one of embodiments 148-157, further comprising an antioxidant.

159. The cell culture medium of embodiment 158, wherein the antioxidant comprises N-acetyl cysteine, nicotinamide, or a combination thereof.

160. The cell culture medium of any one of embodiments 148-159, further comprising a cell survival agent.

161. The cell culture medium of embodiment 160, wherein the cell survival agent is a supplement for a serum-free cell medium.

162. The cell culture medium of embodiment 161, wherein the supplement comprises a B27 supplement, an N2 supplement, or a combination thereof.

163. The cell culture medium of embodiment 161 or 162, wherein the supplement does not comprise vitamin A.

164. The cell culture medium of any one of embodiments 148-163, wherein the FGF comprises FGF2, FGF3, FGF10, FGF22, or a combination thereof.

165. The cell culture medium of embodiment 164, wherein the FGF comprises FGF10.

166. The cell culture medium of any one of embodiments 148-165, wherein the TGF-beta inhibitor comprises an inhibitor of ALK5.

167. The cell culture medium of any one of embodiments 148-166, wherein the TGF-beta inhibitor comprises A83-01.

168. The cell culture medium of any one of embodiments 148-167, further comprising an amino acid supplement.

169. The cell culture medium of embodiment 168, wherein the amino acid supplement comprises an NEAA supplement, an L-glutamine substitute, or a combination thereof.

170. The cell culture medium of any one of embodiments 148-169, further comprising a serum replacement component.

171. The cell culture medium of embodiment 170, wherein the serum replacement component comprises KOSR.

172. The cell culture medium of any one of embodiments 148-171, further comprising a buffering agent.

173. The cell culture medium of embodiment 172, wherein the buffering agent comprises HEPES. 174. Use of the cell culture medium of any one of embodiments 148-173 for expanding a population of hepatocytes.

175. A method of culturing hepatocytes, the method comprising culturing one or more hepatocytes on a cell culture surface in the presence of a cell culture medium comprising a basal cell culture medium for human cells to which is added a concentration of:
an FGF,
an EGF,
an HGF,
an R-spondin, and
a TGF-beta inhibitor,
wherein one or more of FGF7, Wnt3a, and TGF-alpha are not added to the cell culture medium, wherein the surface of the cell culture vessel is coated with an ECM, and wherein the hepatocytes are cultured in a two-dimensional culture system.

176. The method of embodiment 175, wherein one or more of alanine, asparagine, aspartic acid, glutamic acid, glycine, proline, or serine are added to the cell culture medium.

177. The method of embodiment 175, wherein:
(a) the concentration of the FGF in the cell culture medium is 5 ng/ml to 100 ng/ml;
(b) the concentration of the EGF in the cell culture medium is 10 ng/ml to 100 ng/mL;
(c) the concentration of the R-spondin in the cell culture medium is 5 ng/ml to 150 ng/ml;
(d) the concentration of the HGF in the cell culture medium is 10 ng/ml to 50 ng/ml; or
(e) the TGF-beta inhibitor comprises A83-01, wherein the concentration of A83-01 in the cell culture medium is 1 μM to 5 μM.

178. The method of embodiment 175, wherein a serum or a serum replacement component is added to the cell culture medium at a volumetric concentration of 1% to 6% and is increased to a volumetric concentration of 7% to 15% when the cell density of the hepatocytes reaches 30% to 70% confluency.

179. The method of embodiment 175, wherein the cell culture vessel is coated with an ECM at a density of 0.1 μg/cm² to 0.7 μg/cm².

180. The method of embodiment 175, wherein the ECM comprises a laminin.

181. The method of embodiment 175 wherein the ECM does not include a hydrogel or a three-dimensional matrix.

182. The method of embodiment 175, wherein a B27 supplement, an N2 supplement, N-acetyl cysteine, or a combination thereof is added to the cell culture medium.

183. The method of embodiment 175, wherein the hepatocytes are PHHs or are derived from reprogrammed cells.

184. The method of embodiment 183, wherein the reprogrammed cells are reprogrammed iPSCs or wherein the hepatocytes are terminally differentiated hepatocytes.

185. A method of culturing hepatocytes, the method comprising culturing one or more hepatocytes in contact with an ECM in the presence of a cell culture medium comprising a basal cell culture medium for human cells to which is added a concentration of:
an FGF,
an EGF,
an HGF,
an R-spondin,
and a TGF-beta inhibitor,
wherein one or more of FGF7, Wnt3a, and TGF-alpha and one or more of gastrin, arachidonic acid, and prostaglandin E2 are not added to the cell culture medium.

186. The method of embodiment 185, wherein FGF7, Wnt3a, and TGF-alpha are not added to the cell culture medium.

187. The method of embodiment 185, wherein gastrin, arachidonic acid, and prostaglandin E2 are not added to the cell culture medium.

188. The method of embodiment 185, wherein:
(a) the TGF-beta inhibitor does not comprise Noggin;
(b) the FGF comprises FGF10; or
(c) the R-spondin comprises R-spondin 1.

189. The method of embodiment 185, wherein:
(a) the concentration of the FGF in the cell culture medium is 5 ng/ml to 100 ng/ml;
(b) the concentration of the EGF in the cell culture medium is 10 ng/ml to 100 ng/mL;
(c) the concentration of the R-spondin in the cell culture medium is 5 ng/ml to 150 ng/ml;
(d) the concentration of the HGF in the cell culture medium is 10 ng/ml to 50 ng/ml; or
(e) the TGF-beta inhibitor comprises A83-01, wherein the concentration of A83-01 in the cell culture medium is 1 μM to 5 μM.

190. The method of embodiment 185, wherein one or more of alanine, asparagine, aspartic acid, glutamic acid, glycine, proline, or serine are added to the cell culture medium.

191. The method of embodiment 185, wherein the ECM is coated on a two-dimensional surface or wherein the ECM promotes cell adhesion to a surface of a cell culture vessel and wherein the hepatocytes are cultured in a two-dimensional culture system.

192. The method of embodiment 185, wherein the hepatocytes are PHHs or derived from reprogrammed cells.

193. The method of embodiment 192, wherein the reprogrammed cells are reprogrammed iPSCs or wherein the hepatocytes are terminally differentiated hepatocytes.

194. A method of culturing hepatocytes in contact with an ECM in the presence of a cell culture medium,
wherein the hepatocytes are plated and expanded, wherein a serum or serum replacement component is added to the cell culture medium at a volumetric concentration of 1% to 6% upon plating the hepatocytes and is increased in the cell culture medium to a volumetric concentration of 7% to 15% over the course of culturing, and wherein the cell culture medium comprises a basal cell culture medium for human cells to which is added a concentration of:
an FGF,
an EGF,
an HGF,
an R-spondin, and
a TGF-beta inhibitor,
wherein one or more of FGF7, Wnt3a, and TGF-alpha are not added to the cell culture medium.

195. The method of embodiment 194, wherein the method comprises passaging the hepatocytes, wherein each passage is 5 to 25 days.

196. The method of embodiment 194, wherein the serum or serum replacement component is increased to a volumetric concentration of 7% to 15% when the cell density of the hepatocytes reaches 30% to 70% confluency.

197. The method of embodiment 194, wherein FGF7, Wnt3a, and TGF-alpha are not added to the cell culture medium.

198. The method of embodiment 194, wherein one or more of alanine, asparagine, aspartic acid, glutamic acid, glycine, proline, or serine are added to the cell culture medium.

199. The method of embodiment 194, wherein the ECM comprises a laminin.

200. The method of embodiment 194, wherein the ECM is coated on a two-dimensional surface or wherein the ECM promotes cell adhesion to a surface of a cell culture vessel and wherein the hepatocytes are cultured in a two-dimensional culture system.

201. The method of embodiment 194, wherein the hepatocytes are PHHs or are derived from reprogrammed cells.

202. The method of embodiment 201, wherein the reprogrammed cells are reprogrammed iPSCs or wherein the hepatocytes are terminally differentiated hepatocytes.

203. The method of embodiment 194, wherein:
(a) the concentration of the FGF in the cell culture medium is 5 ng/ml to 100 ng/ml;
(b) the concentration of the EGF in the cell culture medium is 10 ng/ml to 100 ng/ml;
(c) the concentration of the R-spondin in the cell culture medium is 5 ng/ml to 150 ng/ml;
(d) the concentration of the HGF in the cell culture medium is 10 ng/ml to 50 ng/ml; or
(e) the TGF-beta inhibitor comprises A83-01, wherein the concentration of A83-01 in the cell culture medium is 1 μM to 5 μM.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of expanding culturing hepatocytes, the method comprising culturing one or more hepatocytes on a cell culture surface in the presence of a cell culture medium comprising a basal cell culture medium for human cells to which is added a concentration of:
a fibroblast growth factor (FGF),
an epidermal growth factor (EGF),
a hepatocyte growth factor (HGF),
an R-spondin, and
a transforming growth factor-beta (TGF-beta) inhibitor,
wherein one or more of FGF7, Wnt3a, and transforming growth factor-alpha (TGF-alpha) are not added to the cell culture medium, wherein the surface of the cell culture vessel is coated with an extracellular matrix (ECM), and wherein the hepatocytes are cultured in a two-dimensional culture system, thereby expanding the hepatocytes.

2. The method of claim 1, wherein one or more of alanine, asparagine, aspartic acid, glutamic acid, glycine, proline, or serine are added to the cell culture medium.

3. The method of claim 1, wherein:
(a) the concentration of the FGF in the cell culture medium is 5 ng/ml to 100 ng/ml;
(b) the concentration of the EGF in the cell culture medium is 10 ng/ml to 100 ng/ml;
(c) the concentration of the R-spondin in the cell culture medium is 5 ng/ml to 150 ng/ml;
(d) the concentration of the HGF in the cell culture medium is 10 ng/ml to 50 ng/ml; or
(e) the TGF-beta inhibitor comprises A83-01, wherein the concentration of A83-01 in the cell culture medium is 1 μM to 5 μM.

4. The method of claim 1, wherein a serum or a serum replacement component is added to the cell culture medium at a volumetric concentration of 1% to 6% and is increased to a volumetric concentration of 7% to 15% when the hepatocytes reach 30% to 70% confluency.

5. The method of claim 1, wherein the cell culture vessel is coated with an ECM at a density of 0.1 μg/cm² to 0.7 μg/cm².

6. The method of claim 1, wherein the ECM comprises a laminin.

7. The method of claim 1, wherein the ECM does not include a hydrogel or a three-dimensional matrix.

8. The method of claim 1, wherein N-acetyl cysteine is added to the cell culture medium.

9. The method of claim 1, wherein the hepatocytes are primary human hepatocytes (PHHs) or are derived from reprogrammed cells.

10. The method of claim 9, wherein the reprogrammed cells are reprogrammed induced pluripotent stem cells (iPSCs) or wherein the hepatocytes are terminally differentiated hepatocytes.

11-12. (canceled)

13. The method of claim 1, wherein one or more of gastrin, arachidonic acid, and prostaglandin E2 are not added to the cell culture medium.

14. The method of claim 1, wherein:
(a) the TGF-beta inhibitor does not comprise Noggin;
(b) the FGF comprises FGF10; or
(c) the R-spondin comprises R-spondin 1.

15-19. (canceled)

20. A method of expanding hepatocytes in contact with an ECM in the presence of a cell culture medium, the method comprising:
(a) culturing hepatocytes in a culture medium, wherein a serum or serum replacement component is added to the cell culture medium at a volumetric concentration of 1% to 6%; and
(b) increasing the volumetric concentration of serum or serum replacement component to a volumetric concentration of 7% to 15% over the course of culturing,
wherein the cell culture medium comprises a basal cell culture medium for human cells to which is added a concentration of:
an FGF,
an EGF,
an HGF,
an R-spondin, and
a TGF-beta inhibitor,
wherein one or more of FGF7, Wnt3a, and TGF-alpha are not added to the cell culture medium, thereby expanding the hepatocytes.

21. The method of claim 20, wherein the method comprises passaging the hepatocytes, wherein each passage is 5 to 25 days.

22. The method of claim 20, wherein increasing the volumetric concentration of serum or serum replacement component to a volumetric concentration of 7% to 15% of step (b) is performed when the hepatocytes reach 30% to 70% confluency.

23. The method of claim 20, wherein FGF7, Wnt3a, and TGF-alpha are not added to the cell culture medium.

24. The method of claim 20, wherein one or more of alanine, asparagine, aspartic acid, glutamic acid, glycine, proline, or serine are added to the cell culture medium.

25. The method of claim 20, wherein the ECM comprises a laminin.

26. The method of claim 20, wherein the ECM is coated on a two-dimensional surface or wherein the ECM promotes cell adhesion to a surface of a cell culture vessel and wherein the hepatocytes are cultured in a two-dimensional culture system.

27. The method of claim 20, wherein the hepatocytes are PHHs or are derived from reprogrammed cells.

28. The method of claim 27, wherein the reprogrammed cells are reprogrammed iPSCs or wherein the hepatocytes are terminally differentiated hepatocytes.

29. The method of claim 20, wherein:
(a) the concentration of the FGF in the cell culture medium is 5 ng/ml to 100 ng/ml;
(b) the concentration of the EGF in the cell culture medium is 10 ng/ml to 100 ng/ml;
(c) the concentration of the R-spondin in the cell culture medium is 5 ng/ml to 150 ng/ml;
(d) the concentration of the HGF in the cell culture medium is 10 ng/ml to 50 ng/ml; or
(e) the TGF-beta inhibitor comprises A83-01, wherein the concentration of A83-01 in the cell culture medium is 1 μM to 5 μM.

30. The method of claim 13, wherein gastrin, arachidonic acid, and prostaglandin E2 are not added to the cell culture medium.

31. A method of expanding hepatocytes, the method comprising culturing one or more hepatocytes on a cell culture surface in the presence of a cell culture medium comprising a basal cell culture medium for human cells to which is added a concentration of:
an FGF,
an EGF,
an HGF,
an R-spondin, and
a TGF-beta inhibitor,
wherein one or more of FGF7, Wnt3a, and TGF-alpha are not added to the cell culture medium, wherein the surface of the cell culture vessel is coated with an ECM, and wherein all of the hepatocytes contact the surface of the cell culture vessel, thereby expanding the hepatocytes.

32. The method of claim 31, wherein:
(a) the concentration of the FGF in the cell culture medium is 5 ng/ml to 100 ng/ml;
(b) the concentration of the EGF in the cell culture medium is 10 ng/ml to 100 ng/ml;
(c) the concentration of the R-spondin in the cell culture medium is 5 ng/ml to 150 ng/ml;
(d) the concentration of the HGF in the cell culture medium is 10 ng/ml to 50 ng/ml; or
(e) the TGF-beta inhibitor comprises A83-01, wherein the concentration of A83-01 in the cell culture medium is 1 μM to 5 μM.

33. The method of claim 31, wherein the cell culture vessel is coated with an ECM at a density of 0.1 μg/cm² to 0.7 g/cm².

34. The method of claim 31, wherein a serum or a serum replacement component is added to the cell culture medium at a volumetric concentration of 1% to 6% and is increased to a volumetric concentration of 7% to 15% when the hepatocytes reach 30% to 70% confluency.

35. The method of claim 1, wherein:
(a) the TGF-beta inhibitor does not comprise Noggin;
(b) the FGF comprises FGF10; or
(c) the R-spondin comprises R-spondin 1.
